(19) United States
(12) Patent Application Publication
(10) Pub. No.: US 2007/0072798 A1
(43) Pub. Date: Mar. 29, 2007

(54) Method for treatment of cardiovascular and metabolic diseases and detecting the risk of the same

(75) Inventors: Jukka T. Salonen, Kuopio (FI); Boryana Todorova, Kuopio (FI); Juha-Matti Aalto, Siilinjarvi (FI); Outi Kontkanen, Kuopio (FI); Mia Pirskanen, Kuopio (FI); Pekka Uimari, Kuopio (FI)

Correspondence Address: BIRCH STEWART KOLASCH & BIRCH, PO BOX 747, FALLS CHURCH, VA, 22040-0747, US

(73) Assignee: Oy Jurilab Ltd, Kuopio (FI)

(21) Appl. No.: 11/483,679

(22) Filed: Jul. 11, 2006

Related U.S. Application Data
(60) Provisional application No. 60/698,040, filed on Jul. 12, 2005.

(51) Int. Cl. A61K 48/00; A61K 38/55

(52) U.S. Cl. 514/12; 514/44

(57) ABSTRACT

This invention relates to the therapeutic, diagnostic and pharmacogenetic use of nucleic acids and proteins involved in human proteolytical system such as serine and cysteine proteases and their inhibitors and pharmaceutical agents and other therapies affecting these. This invention discloses methods for the treatment and prevention of cardiovascular diseases such as coronary heart disease (CHD), acute myocardial infarction (AMI), chronic CHD, arterial hypertension (HT) and cerebrovascular stroke and metabolic disorders such as the metabolic syndrome (MBO) and obesity and methods for detecting or diagnosing a risk of, or predisposition to the said diseases in a subject, for selecting treatment in a subject and for selecting subjects for studies testing cardiovascular, anti-diabetic and anti-obesity drugs, as well as to transgenic animals.

### FIELD OF THE INVENTION

The present invention relates generally to the field of treatment and diagnosis of cardiovascular diseases such as coronary heart disease (CHD), acute myocardial infarction (AMI), arterial hypertension (HT), and metabolic disorders such as the metabolic syndrome (MBO) and obesity. More particularly, it provides new methods for prevention and treatment of CHD, AMI, HT, MBO and obesity. The invention also relates to novel methods for risk assessment, diagnosis and prognosis of CHD, AMI, HT, MBO and obesity. Specifically, the invention is directed to a method that comprises the steps of providing a biological sample of the subject to be tested and detecting the presence or absence of one or several biomarkers in the biological sample. Furthermore, the invention utilises both genetic and phenotypic information as well as information obtained by questionnaires to construct a score that provides the probability of developing CHD, AMI, HT, MBO and obesity. In addition, the invention provides kits to perform the method. The kits can be used to set an etiology-based diagnosis of CHD, AMI, HT, MBO and obesity for targeting of treatment and preventive interventions as well as stratification of the subject in clinical trials testing drugs and other interventions.

### DESCRIPTION OF RELATED ART

Classification and Definitions of CVD, HT and Obesity

Cardiovascular Diseases (CVD) (ICD/10 codes I00-I99, Q20-Q28) include ischemic (coronary) heart disease (CHD), hypertensive diseases, cerebrovascular disease (stroke) and rheumatic fever/rheumatic heart disease, among others (AHA, 2004). In terms of morbidity, mortality and cost CHD is the most important disease group of CVD. CHD (ICD/10 codes I20-I25) includes acute myocardial infarction (AMI), other acute ischemic (coronary) heart disease, angina pectoris; atherosclerotic cardiovascular disease and all other forms of chronic ischemic heart disease (AHA, 2004). Here, acute coronary events, though not technically AMI, are included under the term “AMI”. AMI and angina pectoris are often caused by coronary atherosclerosis, but not always. Other, often contributory pathophysiologies include coronary thrombosis and contriction or contraction and severe arrhythmias. These may cause an AMI also without coronary narrowing by atherosclerosis.

Hypertension (ICD/10 I10-I15) is currently defined as systolic pressure of 140 mmHg or higher or diastolic pressure of 90 mmHg or higher or taking antihypertensive medicine (AHA, 2004). Apart from being a cardiovascular disease (CVD) itself, hypertension is a major risk factor for other CVDs, such as coronary heart disease (CHD), stroke and congestive heart failure (CHF). About half of people who have a first heart attack and two-thirds who have a first stroke have blood pressure (BP) level higher than 160/95 mm Hg. Hypertension precedes the development of CHF in 91% of cases (AHA, 2004).

As the direct measurement of body fat is difficult, Body Mass Index (BMI), a simple ratio of weight to the square of height (kg/m²), is typically used to classify overweight and obese adults. Obesity is most often defined as body-mass index (weight in kg per the square of height in meters) of 30 or more and overweight as BMI of 25 or more but less than 30 (WHO).

Hypertension

Besides some well established, but rather rare forms of secondary hypertension, essential hypertension is the most common diagnosis. Essential hypertension refers to a lasting increase in BP with heterogeneous genetic and environmental causes. It affects 25% of most adult populations (Hasimu et al. 2003) and its prevalence rises with age, irrespective of the type of BP measurement and the operational thresholds used for diagnosis. It aggregates with other cardiovascular risk factors, such as abdominal obesity, dyslipidaemia, glucose intolerance, hyperinsulinaemia, and hyperuricaemia, possibly because of a common underlying cause (Salonen et al. 1981, 1998, Staessen et al. 2003).

The exact pathophysiology or underlying mechanisms responsible for essential hypertension are not fully understood but a variety of factors and regulatory systems have been implicated in its causation and progression (Luft 2001).

Nuclear family studies show greater similarity in BP within families than between families, with heritability estimates ranging between 0.20 and 0.46 (Fuentes RM, 2003). Twin studies document greater concordance of BP in monozygotic than dizygotic twins, giving the highest heritability estimates between 0.48 and 0.64 (Fuentes RM, 2003). Adoption studies demonstrate greater concordance of BP among biological siblings than adoptive siblings living in the same household, estimating heritability between 0.45 and 0.61 (Fuentes RM, 2003). Heritability of HT is commonly estimated as 40-70%.

The genetic background of essential hypertension is complex and currently not fully understood (Naber and Siffert, 2004). Clearly both genetic and environmental factors play highly significant roles, ultimately resulting in sufficient abnormalities in gene expression (over-, under-, zero, or defective production) to yield the pathological elevations of blood pressure. In most cases a combination of abnormal expression from multiple genes likely yields the final deleterious phenotype (Garbers and Dubois, 1999).

Obesity

Obesity is a complex, multi-factorial chronic disease involving environmental (social and cultural), genetic, physiologic, metabolic, behavioral and psychological components. It is the second leading cause of preventable death in the U.S. Although obesity is not a recent phenomenon as the historical roots of obesity can be traced back to 25,000 years ago, the epidemic of obesity is a global health issue across all age groups, especially in industrialized countries (American Obesity Association, 2006).

Obesity is an excessive accumulation of energy in the form of body fat impairing health. The degree of health impairment is determined by three factors: 1) the amount of fat 2) the distribution of fat and 3) the presence of other risk factors (NIH, 1998).

Although BMI provides a simple convenient measurement of obesity, a more important aspect of obesity is the regional distribution of excess body fat. Mortality and morbidity vary with the distribution of body fat, with the highest risk linked to excessive abdominal fat (‘central obesity’) (Macdiarmid, 1998).

Twin studies suggest a heritability of fat mass of between 40% and 70% with a concordance of 0.7-0.9 between monozygotic twins compared to 0.35-0.45 between dizygotic twins (Stunkard et al. 1986, 1990, Allison et al. 1996, Maes et al. 1997).

MBO

The term metabolic syndrome is used to describe a concurrence of disturbed glucose and insulin metabolism, overweight and abdominal fat distribution, mild dyslipidemia and hypertension. The syndrome is characterized by insulin resistance, and is also known as the insulin resistance syndrome. World Health Organization (WHO) consultation for the classification of diabetes and its complications (Alberti KG, 1998) and the National Cholesterol Education Program (NCEP, 2001) Expert Panel have recently published definitions of the metabolic syndrome.

All of the features, which are characteristic for MBO are risk factors for atherosclerosis. Thus MBO constitutes a significant risk for a cardiovascular outcome, such as CHD and stroke. MBO with its complications is a syndrome in which most of the body systems are involved. All metabolic and signaling pathways involved in the development of MBO and its complications are not known at the moment.

Proteases and Proteinases and their Inhibitors

Proteolytic enzymes comprise a group of structurally and functionally diverse proteins that have the common ability to catalyze the hydrolysis of peptide bonds (Barrett A J etal 1998, Hooper N M, 2002). A number of important processes that regulate the activity and fate of many proteins are strictly dependent on proteolytic processing events. These include the ectodomain shedding of cell surface proteins; the appropriate intra- or extracellular localization of multiple proteins; the activation and inactivation of cytokines, hormones and growth factors; the regulation of transcription factor activity; or the exposure of cryptic neoproteins with functional roles distinct from the parent molecule from which they derive after proteolytic cleavage reactions (Lopez-Otin C and Overall CM, 2002). These protease-mediated processing events, which are distinct from nonspecific protein degradation reactions, are vital in the control of essential biological processes such as DNA replication, cell cycle progression, cell proliferation, differentiation and migration, morphogenesis and tissue remodeling, immunological reactions, ovulation, fertilization, neuronal outgrowth, angiogenesis, hemostasis, and apoptosis.

Almost two percent of the human genome is estimated to code for proteases (Southan C, 2001). Thus, proteolysis and other actions of proteases occur widely enough in the human body to be of etiologic and pathophysiologic importance in common chronic diseases.

A nomenclature to describe the interaction of a substrate with a protease, and thus protease specificity, has been introduced in 1967 by Schechter and Berger. In this system, it is considered that the catalytic site of the enzyme is flanked on one or both sides by specific subsites, called S (for subsites), each able to accommodate the sidechain of a single amino acid residue, called P (for peptide). The sites are numbered as S1 to Sn towards the N-terminus and S1′ to Sn′ towards the C-terminus, S1 and S1′ situated nearest the catalytic side of the enzyme. The amino acid residues of the N-terminal side of the scissile bond (the peptide bond to be cleaved) are numbered P1 . . . Pn and of the C-terminal side P1′ . . . Pn′, as the P1 or P1′ residues are those located near the scissile bond (Schechter I and Berger A, 1967).

According to the Nomenclature Committee of the International Union of Biochemistry and Molecular Biology (NC-IUBMB), dependent on the site of action, peptidases are divided into exopeptidases, acting near a terminus of a polypeptide chain, and endopeptidases (proteinases) that act internally in polypeptide chains. On the basis of the mechanism of catalysis, proteinases form five distinct subclasses: aspartic (EC 3.4.23), metalloproteinases (EC 3.4.24), cysteine (EC 3.4.22), serine (EC 3.4.21) and threonine proteinases (EC 3.4.25). A sixth subclass of proteinases is formed by those, which have not been assigned to any of the mentioned above (EC 3.4.99).

Peptide proteinase inhibitors can be found as single domain proteins or as single or multiple domains within proteins; these are referred to as either simple or compound inhibitors, respectively. In many cases they are synthesized as part of a larger precursor protein, either as a prepropeptide or as an N-terminal domain associated with an inactive peptidase or zymogen. Removal of the N-terminal inhibitor domain either by interaction with a second peptidase or by autocatalytic cleavage activates the zymogen.

The serine proteinases are characterized by the unique catalytic triad of Ser, Asp and His (Steinhoff M et al, 2005). Produced as inactive precursors-zymogens, they undergo a process of activation, “limited proteolysis” or zymogen activation. The serine proteinases exhibit various substrate specificities, related to the amino acid sequences in the diverse enzyme subsites interacting with the substrate residues. Members of the serine proteinases family such as chymotrypsin, trypsin, thrombin, cathepsin G, coagulation factors VIIa, IXa, XIa and XIIa, plasma and tissue kallikreins, etc. imply for the high biological importance in health and disease for both peptidases and their inhibitors.

The group of the cysteine proteinases which constitutes of a number of cathepsins, calpain-1 and calpain-2, caspase-1 and others plays a major role in intracellular lysosomal protein degradation, as well as in the extracellular protein degradation and turnover. The cysteine proteinases, like the serine proteinases, require processing in order to convert to the active enzyme form. The process of removal of the amino-terminal region (proregion) plays important role not only as an inhibitor of the enzymatic activity but also as a corrective for the folding and protection of the newly formed protein (Oliveira A S et al, 2003).

Despite of their life-promoting fiction and precise cellular control of biological processes, proteases have also highly damaging potential, thus, a strict management on their temporal and spatial activity is essential. Several mechanisms exist one of which, for instance, is the presence of conserved prodomains in proteases that serve an auto-inhibitory role to prevent activation at the wrong place or time and they are often required as intramolecular chaperones during protein synthesis and folding (Oliveira A S et al, 2003). The prodomains can also function as a contact face for cell-surface receptors and to direct proteases to specific substrates or locations in a tissue (Oliveira A S et al, 2003).

Proteins inhibiting the protease activity are very important regulators of enzyme activity of proteases. A broad definition of a protease inhibitor incorporates proteins, which have the potential to attenuate the activities of peptidases both in vitro and in vivo by the formation of complexes (Puente X S et al, 2003). The ways in which the inhibitors interact with their target enzymes vary enormously, but two general types of inhibition are recognized: reversible tight-binding reactions and irreversible “trapping” reactions, for which the inhibitor can be described as a suicide inhibitor (Puente X S et al, 2003). Three families of protease inhibitors are showing irreversible mode of inhibiting, one of which is the family of serpins.

Serpins

The serpins (serine proteinase inhibitors) are a superfamily of proteins (350-500 amino acids in size) that fold into a conserved structure and employ a unique suicide substrate-like inhibitory mechanism (Silverman et al. 2001). They regulate diverse physiological processes (Janciauskiene 2001, van Gent et al. 2003). Serpins exhibit conformational polymorphism shifting from native to cleaved, latent, delta, or polymorphic forms. Many serpins, i. e., antitrypsin and antichymotrypsin, function as serine protease inhibitors which regulate blood coagulation cascades. Non-inhibitory serpins perform many diverse functions such as chaperoning proteins or transporting hormones. Serpins are of medical interest because mutations can cause blood clotting disorders, emphysema, cirrhosis, and dementia.

The serpins and related proteins constitute one of the earliest described protein superfamilies recognized by Hunt and Dayhoff in 1981 by computer analysis of amino acid sequence identity (Scott et al. 1999). Most serpins are secreted and attain physiologic concentrations in the blood and extracellular fluids. However, a subset of the serpin superfamily, the ov-serpins, also resides intracellularly (Askew et al. 2001).

The serpin superfamily contains over 500 members in a variety of species including animals, viruses and plants with molecular weight of 40-50 kDa (Scott et al. 1999, Janciauskiene 2001). In human plasma they represent approximately 2% of the total protein (van Gent et al. 2003). Family members are easily identified by amino acid sequence alignments due to their high degree of structural conservation. The serpin tertiary structure consists of three β-sheets, approximately nine α-helices, and several loops that are arranged into a metastable conformation. Serpins employ a unique suicide-substrate-like inhibitory mechanism to neutralise their target proteinases. The mobile reactive site loop (rsL), which is perched on the surface of the molecule, serves as the pseudo-substrate and binds to the active site of the proteinase. Upon rsL cleavage, the serpin undergoes a major conformational rearrangement that traps the proteinase in a covalent acyl-enzyme intermediate (Scott et al. 1999).

The molecular structure and physical properties of serpins permit these proteins to adopt a number of variant conformations under physiological conditions including the native inhibitory form and several inactive, non-inhibitory forms, such as complexes with protease or other ligands, cleaved, polymerised and oxidised (Janciauskiene 2001).

In 1993 amino acid similarities among chicken ovalbumin (ov), PAI2 (SERPINB2), and MNEI (SERPINB1) led to the identification of a subgroup of the serpin superfamily referred to as Ov-serpins (B Clade). The ov-serpins differ from the archetypal fluid phase (circulatory) serpins such as α1-antitrypsin (SERPINA1) or antithrombin III (SERPINC1) by the lack of a cleavable N-terminal secretory signal peptide, the absence of N- and C-terminal extensions, serine (Ser) instead of asparagine (Asn) at the penultimate position (Askew et al. 2001, Silverman et al. 2001, Scott et al. 1999) and a variable residue rather than valine at position 388 (Scott et al. 1999).

The human ov-serpins are divided into two classes depending on whether exon 3 encodes for an extra loop (CD loop) between helices C and D. Those ov-serpins containing a CD loop have an additional intron that interrupts exon 3. The net result is that the human ov-serpins contain either seven or eight exons. In both classes of genes, the translational start sites are located in exon 2 (Askew et al. 2001, Silverman et al. 2001, Scott et al. 1999). Intron/exon splice site phasing is conserved in ov-serpins and has been used to predict evolutionary relatedness of members of the serpin superfamily. The six introns (A, B, D, E, F, and G) found in the ov-serpins structure occur in conserved locations. Intron C, registered in a subset of ov-serpins, is located in the C-D interhelical loop and its exact location is not conserved among serpins.

Unlike ovalbumin itself, most ov-serpins reside intracellularly with a cytoplasmic or nucleocytoplasmic distribution. However, several ov-serpins (PAI2, megsin (SERPINB7), MNEI, maspin (SERPINB5), and the SCCAs (SERPINB3 and 4)) may function extracellularly as they are released from cells under certain conditions. Release may be facilitated by an embedded, noncleaved hydrophobic N-terminal signal sequence and appears to involve both conventional and non-endoplasmic reticulum-Golgi secretory pathways. Regardless of how ov-serpins are released from cells, those with rsL cysteine or methionine residues are susceptible to oxidative inactivation and are likely to have a limited half-life in the extracellular milieu (Silverman et al. 2001).

Serpin dysfunction has been previously implicated in thrombosis, emphysema, cirrhosis, immune hypersensitivity, mental disorders and in diseases characterised by connective and other tissue self-destruction (Janciauskiene 2001, Scott et al. 1999), but to the best of our knowledge, not to CHD, AMI, HT, MBO and obesity.

SPINK Genes

Another group of protease/proteinase inhibitors is characterised by common Kazal type domain structure. The Serine Protease Inhibitor, Kazal type 5 (SPINK5), the Serine Protease Inhibitor Kazal type 5-like 3 (SPINK5L3) and the Serine Protease Inhibitor Kazal type 5-like 2 (SPINK5L2) genes are found in a cluster on the 5q32 cytogenetic region of the 5^{th} chromosome. This region has been reported to be associated with hereditary disorders such as Netherton disease (OMIM: 256500), as well as conditions related to immune system reactivity such as type 1 diabetes (OMIM: 605598), susceptibility/resistance to S. Mansoni infection (OMIM: 181460) or malaria (OMIM: 248310), and atopic dermatitis (OMIM: 605845). Besides the SPINK5, SPINK5L2 and SPINK5L3 genes, there are other representatives of the Serine Protease Inhibitor Kazal type (SPINK) genes in the 5q32 region, the SPINK1, SPINK5L1 and SPINK6 genes.

SPOCK Genes

SPOCK family genes are extracellular multidomain glycoproteins with unknown function. The first protein member encoded by the SPOCK family genes, testican-1, was identified from human seminal plasma, and other proteins, testican 2 and testican 3, have been identified by homology. All testicans have similar protein domain structure, including a signal peptide, a unique N-terminal testican-family specific sequence, a follistatin domain, an extracellular calcium-binding domain, a thyroglobulin domain, and a carboxyterminal region with putative glycosaminoglycan binding sites. For example, a splice variant of testican 3, N-Tes, lacks the carboxyterminal thyroglobulin domain and putative glycosaminoglycan binding sites. Testicans are highly expressed in neurons.

Three members of testican family genes, testicans 1, 2 and 3, have been found to date by cDNA cloning, with different polypeptide lengths. Their protein structures, although similar, are the most different in carboxyterminal regions. Human testicans 2 and 3 are located in chromosome 10pter-q25.3 and chromosome 4q32.3, respectively. Testicans form a subgroup within the BM-40/SPARC/osteonectin family of modular extracellular proteins (Hartmann U and Maurer P, 2001).

Even though CHD, HT and obesity have a high heritability, only a part of the genes contributing to their causation are known to date. This suggests that there are also unknown pathways and molecular mechanisms acting in their etiology. As all these diseases are common in most populations, it is highly assumable that there must be common defects or traits causing them. Alternatively or in addition, biochemical phenomena that occur widely in different organs, tissues and cells are likely to contribute. Such are determined by the genes and proteins of this invention.

Tolloid-Like Genes

Scott et al. (1999) compared enzymatic activities and expression domains of 4 mammalian BMP1/TLD-like proteases and found differences in their ability to process fibrillar collagen precursors and to cleave chordin. As previously demonstrated for BMP1 and TLD, TLL1 specifically processes procollagen C-propeptides at the physiologically relevant site, whereas TLL2 lacks this activity. BMP1 and TLL1 cleave chordin, at sites similar to procollagen C-propeptide cleavage sites, and counteract dorsalizing effects of chordin upon overexpression on Xenopus embryos. Proteases TLD and TLL2 do not cleave chordin.

Tolloid-like-1 (TLL1) is an astacin-like metalloprotease that shares structural similarity to the morphogenetically important proteases bone morphogenetic protein-1 (BMP1) and Drosophila Tolloid (TLD). TLL1 potentiates the activity of the bone morphogenetic proteins (BMPs). There are no previous suggestions in the literature that TLL genes would have any role in either CHD, AMI, HT, MBO or obesity.

### SUMMARY OF THE INVENTION

This invention provides novel methods for the treatment and prevention of cardiovascular diseases CHD, AMI, chronic CHD, HT and cerebrovascular stroke, and for metabolic disorders such as MBO and obesity in a human or an animal.

The invention also provides methods and test kits for risk prediction, diagnosis or prognosis of a cardiovascular or metabolic condition or trait in a subject. It also provides methods and test kits for targeting and monitoring antihypertensive, anti-CHD, anti-diabetic and anti-obesity treatments as well as methods and test kits for stratifying subjects for studies testing antihypertensive, anti-CHD, anti-diabetic and anti-obesity effects of drugs.

Additionally, the invention discloses screening assays, transgenic animals and pharmaceutical compositions to study and develop therapies for CHD, AMI, HT, stroke, MBO and obesity.

Yet another object of the invention are any diagnostic or other imaging methods which are based on the genes or the products of these genes disclosed here. The said gene products may be labeled either with a radioactive or another type of label and imaged by a respective scanner. This technology can be used in the diagnostics of AMI, CHD, hypertension and its sequalae such as cerebrovascular stroke, cardiac failure, congestive heart disease, renal disease or retinal disease. For example, early phases of ischemic damage to myocardium may be detected by imaging changes in the expression of the genes or the levels of the encoded proteins of the present invention in the cardiac muscle.

The invention discloses a novel role for genes and their encoded proteins or polypeptides regulating peptidases and other proteins, and endogenous and exogenous modulators of said genes, proteins or polypeptides. Examples of the genes of this invention are SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK1, SPOCK2, and SPOCK3.

The invention relates further to physiological and biochemical routes and pathways related to these genes. These pathways provide a basis for further research and development of CHD, AMI, HT, stroke, MBO and obesity predisposition, diagnosis and treatment.

The invention helps meet the unmet medical needs in at least two major ways: 1) it defines drug and other therapeutic targets that can be used to screen and develop therapeutic agents and gene therapies that can be used to prevent CHD, AMI, HT, MBO and obesity before they manifest clinically, to prevent complications, treat clinical symptoms and/or retard the progression of CHD, AMI, HT, MBO and obesity in those who have already developed a clinical disease, and 2) it provides a means to define patients at higher risk for CHD, AMI, HT, MBO or obesity than the general population who can be more aggressively managed by their physicians in an effort to prevent CHD, AMI, HT, MBO and/or obesity.

### DETAILED DESCRIPTION OF THE INVENTION

This invention is based on the disclosure that SNP markers within or close to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK1, SPOCK2 and SPOCK3 genes in Eastern Finnish subjects are associated with altered risk of having CHD, AMI, HT, stroke, MBO and/or obesity. Thus altered functions of SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 genes could be related to the pathogenesis of CHD, AMI, HT, stroke, MBO and obesity. All these genes encode proteins, which are able to regulate the activity of peptidases (proteases and/or proteinases) in various organs, tissues and cells as well as extracellularly in the human body.

Disturbance of the balance between proteolytic enzymes (peptidases) and their inhibitors either in cells or in extracellular space e.g. due to accumulation of peptidases or their inhibitors will alter activity of many vital cellular metabolic pathways, processes and functions. This may result in degradation of vital proteins and peptides or accumulation of peptidase substrates in cells and leading to the degeneration of cells. DNA sequence polymorphisms may alter expression profiles of peptidases and their inhibitors causing such changes in cells and extracellular space. An example of this type of condition is inflammation, which may occur in excess to what would be appropriate for the human body to counteract infection, to repair arterial damage etc.

An example of relevant proteolytic enzymes of this invention are those that degrade insulin and thus predispose to diabetes. Yet another example are proteases that degrade vasoactive peptides which affect blood pressure. For instance, accelerated degradation of vasodilating peptides will elevate blood pressure and predispose to hypertension. Both a loss-of-function mutation of an inhibitor of a protease responsible for this and a gain-of-function mutation in the respective protease would elevate blood pressure through the inhibition of a vasodilating peptide. Yet another example is too fast degradation of appetite reducing or controlling peptides, which can lead to excessive appetiute, excess energy intake and thus to obesity, MBO, HT and T2D and their sequelae. Yet another example are proteases and their inhibitors affecting peptides and proteins that mediate the inflammatory response in the body, contributing to the causation of obesity, MBO, HT and T2D and their sequelae.

We propose that genetic defects that either enhance or reduce the function or activity of proteolytic enzyme systems, such as peptidases and/or their endogenous inhibitors or enhancers, is a general mechanism in the body of a mammalian subject, such as human, which contributes to the development of common degenerative diseases and related traits, such as cardiovascular and metabolic diseases and traits predisposing to them. Restoring the balance between peptidases and their endogenous modulators offers novel methods to treat and prevent said common degenerative diseases.

Therefore, we have invented an important new role and industrial use for the SERPIN, SPINK and SPOCK gene and protein families, exemplified by SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 genes and their encoded proteins as described in detail below.

In accordance with the present invention we have discovered a series of biomarkers associated with CHD, AMI, HT, stroke, MBO or/and obesity. “Biomarker” in the context of the present invention refers to an organic biomolecule, particularly to a nucleic acid fragment containing a single nucleotide polymorphism or an expression product of a gene, which is differentially present in a sample taken from subjects (patients) having CHD, AMI, HT, stroke, MBO or/and obesity as compared to a comparable sample taken from subjects who do not have CHD, AMI, HT, stroke, MBO or/and obesity “Organic biomolecule” refers to an organic molecule of biological origin, e.g., steroids, amino acids, nucleotides, sugars, polypeptides, polynucleotides, complex carbohydrates or lipids. A biomarker is differentially present between two samples if the amount or the biological activity of the biomarker in one sample differs in a statistically significant way from the amount or the biological activity of the biomarker in the other sample

Serpins

The empirical evidence, presented below, shows that a number of members of the SERPINB-family genes (the ov-serpins) play a role in cardiovascular and metabolic diseases and related quantitative traits (Tables 2-6, 19-22 and text).

Some A-serpins such as plasminogen activator inhibitor 1 (PAI-1) and angiotensinogen [(Serine (or cysteine) proteinase inhibitor, clade A (Alpha-1 antiproteinase, antitrypsin), member 8] are involved in the development of cardiovascular diseases and common syndromes such as atherosclerosis, diabetes and hypertension (De Taeye B et al, 2004, Naber C K and Siffert W, 2004); marked upregulation of protease-nexin 1—another serpin family member in hypertensive rats (Bouton M C et al, 2003); and discernable alterations in kallikrein-binding protein in hypertension (Chao J et al, 1990).

The irreversibility of proteinase inhibition achieved by the serpins has made them the principal inhibitors controlling both intra- and extracellular proteolytic pathways. They regulate such diverse physiological processes as cascades involved in blood clotting, fibrinolysis, complement activation, cell motility, inflammation, and cell death (Askew et al. 2001, Janciauskiene 2001, Silverman et al. 2001, Scott et al. 1999).

The primary function of most members of the serpin family is to neutralise overexpressed (up-regulated) serine proteinase or protease activity. Any alteration changing the structure and/or expression (synthesis, secretion or degradation) of a serpin may result in pathological changes because of reduced levels of biologically functional serpin. This may lead to enhanced protease activity. An example of this are enzymes that degrade insulin and predispose to diabetes. Yet another example are proteases that degrade vasoactive peptides that affect blood pressure. For instance, accelerated degradation of vasodilating peptides will elevate blood pressure and predispose to hypertension. Yet another example is too fast degradation of appetite reducing or controlling peptides, which can lead to excessive appetiute, excess energy intake and thus to obesity, MBO, HT and T2D and their sequelae.

The variable length loop between helices C and D may confer functional motifs involved in, for example, nuclear localization or transglutamination. Also, most of the ov-serpins appear to reside intracellularly with a cytoplasmic or nuclear-cytoplasmic distribution. To date, 13 human ov-serpins (SERPINB1-13) have been cloned and sequenced. They reside within two chromosomal clusters located at 6p25 and 18q21.3. With the possible exception of SERPINB5, all of the human ov-serpins inhibit various serine or cysteine proteinases and are involved in biological processes such as the inhibition of cell migration, protection against certain programmed cell death pathways and the neutralization of endogenous granule proteinases that leak into the cytosol (Askew et al. 2001, Silverman et al. 2001, Scott et al. 1999, van Gent et al. 2003).

With the possible exception of maspin, all human ov-serpins are functional, competitive inhibitors of serine or cysteine proteinases. Several members of the group inhibit more than one proteinase, and dual reactive sites (utilization of more than one P1 residue) have been described for SERPINB1, SERPINB3, SERPINB4, SERPINB6, SERPINB8 and SERPINB9. However, the CD loops of the ov-serpins have the potential to interact with other proteins. For example, the CD loop of SERPINB2 is required for its cell survival function and is a target for transglutamination (Silverman et al. 2001).

The ability of many ov-serpins to inhibit more than one proteinase and their presence in epithelial cells suggest that they play a role in barrier function or host defense against microbial or viral proteinases. For example, SERPINB9 inhibits Bacillus subtilisin, and SERPINB8 inhibits furin, a subtilisin-related enzyme. Additional known functions of ov-serpins include the regulation of: 1) cell growth or differentiation, as exemplified by the role of megsin in megakaryocyte differentiation, 2) tumor cell invasiveness and motility, as shown by the inhibitory role of maspin in breast and prostate tumors, and 3) angiogenesis (Silverman et al. 2001).

Serpins belong to MEROPS (http://merops.sanger.ac.uk) inhibitor family I4, clan ID and are primarily known as irreversible serine protease inhibitors acting against S1 (INTERPRO entry IPR001254), S8 (INTERPRO entry IPR000209) and C14 (INTERPRO entry IPR002398) families of peptidases. Among the group of S1 are serine proteases such as trypsin, tryptase, kallikrein, thrombin, protein C, uPA, tPA, plasmin, coagulation factors VIla, IXa, Xa, XIa, and XIIa, complement factors 1, B and D, complement components C1 and C2, granzymes A and K, hepsin, prostasin and others. Peptidase family S8, the other substrate for serpins' inhibition, contains the serine endopeptidase subtilisin and its homologues. Family S8, also known as the subtilase family, is the second largest family of serine peptidases, in terms of number of sequences, characterized peptidases and broad involvement in various biological processes. The family is divided into two subfamilies, with subtilisin the type-example for subfamily S8A and kexin the type-example for subfamily S8B. Most members of the family are endopeptidases, but tripeptidyl-peptidase II (TPP-II) is an exopeptidase releasing tripeptides from the N-terminus of peptides (MEROPS).

The apoptosis cascade is primarily controlled by the caspases (Cysteine-dependent ASPartyl-specific proteASE), which form a large part of the C14 proteases-another source for serine inhibition. Apoptosis is a genetically programmed, morphologically distinct and conserved form of cell death, which can be triggered by a variety of physiological and pathological stimuli. The substrate specificities of the individual caspases are distinct, being determined by the residues present in the pockets of P2, P3 and P4. It is considered that the caspases with long prodomains are responsible for the initiation of the apoptotic response whereas those with shorter prodomains are ‘effector’ caspases (Earnshaw W C et al., 1999). The effector caspases are activated by the initiator caspases and are directly responsible for cell death (Thomberry N A and Lazebnik Y, 1998). Activated caspases act as cysteine proteases, using the sulphydryl group of a cysteine side chain for catalysing peptide bond cleavage at aspartyl residues in their substrates. They have two main roles within the apoptosis cascade: as initiators that trigger the cell death process, and as effectors of the process itself. Caspase-mediated apoptosis follows two main pathways, one extrinsic and the other intrinsic or mitochondrial-mediated. The extrinsic pathway involves the stimulation of various tumour necrosis factor (TNF) cell surface receptors on cells targeted to die by various TNF cytokines, produced by cells such as cytotoxic T cells. The activated receptor transmits the signal to the cytoplasm and a death-inducing signalling complex (DISC) with caspase-8 is formed. The subsequent activation of caspase-8 initiates the apoptosis cascade involving caspases 3, 4, 6, 7, 9 and 10. The intrinsic pathway arises from signals that originate within the cell as a consequence of cellular stress or DNA damage. The subsequent activation of caspase-9 initiates the apoptosis cascade involving caspases 3 and 7, among others. At the end of the cascade, caspases act on a variety of signal transduction proteins, cytoskeletal and nuclear proteins, chromatin-modifying proteins, DNA repair proteins and endonucleases that destroy the cell by disintegrating its contents, including its DNA. The different caspases have different domain architectures depending upon where they fit into the apoptosis cascades. A member of C14, besides the caspases, is the FLIP (caspase-8 inhibitory protein) protein which is an apoptosis regulating, possibly functioning as a critical link between cell survival and cell death pathways in mammalian cells.

The existence of different transcripts derived from alternative splicing (often occurring in exon 3), may affect tissue specificity and substrate specificity of an ov-serpin. This could explain the observed multiple targets of the ov-serpins (more than one protease inhibited by one serpin) and variable tissue availability of different ov-serpins.

To our knowledge the members of the ov-serpin family described herein have not been previously suggested to be related to atherosclerosis, cardiovascular disease, coronary heart disease, AMI (with SERPINB9 as an exception), hypertension, cerebrovascular stroke, the metabolic syndrome or obesity. Taking into account the broad and potent inhibitory capacity the ov-serpins have and the important role of proteases inhibited by the ov-serpins in cardiovascular and metabolic diseases, the ov-serpins could have central role in the pathogenesis of CHD, AMI, HT, stroke, MBO and obesity.

The ov-serpins contain a region with a relatively high degree of conservation which is referred to as “structural core” region. Inhibitory serpins possess a high degree of conservation at many key amino acid residues, believed to be necessary for enabling the protein to undergo the so-called “stressed to relaxed” transition- a conformational change, accompanied by the insertion of the remaining reactive site loop into one of the β-sheets, during which serpins form a stable heat-resistant complex with the target protease. Thus, a non-synonymous mutation at such site would probably alter the inhibitory function of the serpins.

In our example, in the SERPINB11 (epipin, SERPINB11d, SERPINB11e, SERPINB11f), serine (or cysteine) proteinase inhibitor, clade B (ovalbumin) gene a common non-synonymous SNP, rs1395266 (SEQ ID: 46), is known with major allele T and minor allele C. A typical allele frequency of the minor allele is 0.26, based on genotyping of 197 individuals. The mutation leads to substitution of the protein residue isoleucine (Ile) by threonine (Thr) in protein position 293. This position is in a domain which is likely to contribute to the inhibitory effect of the protein. We show in tables 2, 3 and 6 that SNPs in the SERPINB11 gene are associated with CHD, AMI, HT, stroke, MBO and obesity.

SERPLNB11 gene is located on chromosome 18 and has been mapped to 18q21.3 cytogenetic region. The protein product referred to as Serpin B11 consists of 392 amino acid residues and has a molecular weight of 44 kDa. PSORT II analysis (http://psort.nibb.acjp) predicts a cellular location of SERPINB11 protein, most likely in the cytoplasm. Less likely possibilities are the cytoskeleton, the nucleus or vacuoles. However, SERPINB11 as other ov-serpins contains a domain, forming a hydrophobic region near the amino terminus, which, though not cleaved, serves as a signal sequence for serpins that enter the extracellular space (Remold-O'Donnell E, 1993).

Data so far indicates that SERPINB11 product is found in blood, as well as in prostate and kidney. Potential substrates of SERPINB11 are e.g. the S1 family of peptidases (trypsin, tryptase, kallikrein, thrombin, protein C, uPA, tPA, plasmin, coagulation factors VIla, IXa, Xa, XIa, and XIIa, complement factors 1, B and D, complement components C1 and C2, granzymes A and K, hepsin, prostasin and possibly others). Other SERPINB11 substrates may be the S8 family of proteases e.g. proteins involved in the lipid metabolism (proprotein convertase 9, site-1 peptidase), ubiquitously expressed processors of prepetides and proteins involved in cell-cell signalling processes. Examples for those are the proprotein convertase 1, processing the proinsulin and proglucagon to their active forms; furin, representing ubiquitous endoprotease activity within constitutive secretory pathways and capable of cleavage at the RX(K/R)R consensus motif; it releases albumin, complement component C3 and von Willebrand factor from their respective precursors; as well as other precursors of different hormones, renin and neuropeptides (MEROPS). Thus dysregulation of a number of the S8 family members would result in changes in lipid metabolism, blood pressure regulation, neurotransmission (implication on cardiac inervation with possible implication on cardiac rhythm) and even broader outcomes (tables 3-5 relate to the SNPs in SERPINB11 gene and their impact on cardiovascular risk factors).

The human Apolipoprotein (a) is a non-protease homolog of the tryptase-alpha. We found an association between SNP markers in the SERPINB11 gene and the serum concentration of apolipoprotein (a) (e.g. Table 4). This suggests that apo (a) may be a target for the prevention and treatment and a diagnostic marker not only in CHD, but also in hypertension, lipid abnormalities and metablic syndrome.

The C14 family of proteases, or most of the caspases are related to the regulation of cellular death, but their role might well be in other processes as well, as for instance in cell differentiation (InterPro). An example of a caspase not having a role in apoptosis is the caspase-1, which is involved in the processing of interleukin 1 beta precursor, which on turn takes place in the inflammatory process (MEROPS). Not lastly, the activation of apoptosis can lead to caspase-1 activation, providing a link between apoptosis and inflammation, such as during the targeting of infected cells. Thus, caspases and their inhibitors have a potential to be targeted for treatment of conditions caused by excessive apoptosis. These include but are not limited to hypertension, ischaemic injure (such as in atherosclerosis, angina pectoris and AMI) and neurodegenerative diseases.

SERPINB1, found also under the names of monocyte/neutrophil elastase inhibitor (MNEI, M/NEI), Leukocyte elastase inhibitor (LEI), elastase inhibitor (EI), protease inhibitor 2 (PI2) and ELANH2 is localised on the 6p25.2 cytogenetic region. It covers 9,68 kb and encodes for a protein of 379 amino acids and molecular weight of 42741 Da. The SERPINB1 gene contains 6 introns and 7 exons (Zeng et al, 1998). It is highly expressed, in a variety of tissues, for instance in heart, kidney, vessels, blood, pulmonary system, pancreas and peripheral nervous system and the protein is localized to intracellular cytoplasm. The Nuclear Factor-kappaB (NF-kB) has been related to the MNEI transcriptional activity (Zeng W and Remold-O'Donnell E, 2000.).

Sequence alignment has indicated cys-344 residue in the P1 active center, which proposes SERPINB1 as a natural cys-serpin, abrogating elastase activity (OMIM:130135). Characterised originally as a fast-acting inhibitor of neutrophil elastase, it has been shown recently that MNEI protein has broader specificity. A study of Cooley J et al, 2001, has revealed that while the cys-344 site at P1 has been accepted as an inhibitory side for elastase-like proteases, the preceeding phe-343 residue is related to inhibitory characteristic against chymotripsin-like proteases. Thus a role in anti-inflammatory processes has been related to MNEI. A recombinant human MNEI has been produced (Cooley J et al, 1998) and administered in vivo (animal studies) in the presence of inflammatory process in lungs.

Presented as aerosol for inhaling, it showed a dramatic decrease in inflammatory injury in relation to Pseudomonas Aeruginosa infection (Woods D E et al, 2005). Therefore, we consider that one possible explanation of the association of SERPINB1 with atherosclerosis and cardiovascular risk factors may be via its association with inflammation providing an apparent opportunity for developing new trerapies exploiting the antiatherogenic properties of MNEI.

SERPINB1 as a member of the ov-serpin entity may well posses inhibitory activity also against caspases, which form the main core of C14 family of proteases. Interestingly, it has been shown that under certain conditions MNEI vividly promotes the apoptotic process (Torriglia A et al, 1998; Altairac S et al, 2003). It has been observed that the usual elastase- inhibiting role of SERPINB1 is replaced by the appearance of DNase II activity. DNase II is a cation-independent acidic endonuclease which degrades DNA in apoptotic cells, observed as chromatin condensation. It has been presented that DNase II originates from SERPINB1 as a result from a posttranslational modification with a shift of the molecular weight of LEI (SERPINB1) under decreased intracellular pH (Altairac S et al, 2003). The LEI/L-DNase II pathway appears to be independent from caspases (D2-7) and the role of LEI might well be a molecular switch between living and apoptotic cells.

As a result, the significant impact of SERPINB1 on the precise regulation of pro- and antiapoptotic cellular state gives further support to our invention concerning the role of SERPINB1 in cardiovascular and metabolic diseases. The programmed cell death or apoptosis is recognized to play role in the normal tissue turnover, embryonal development and tumor regression as well as in a significant number of pathological conditions, including cardiac pathologies especially related to ischemia, ischemia-reperfusion injury, denervation, etc. As endothelial cells are important determinants of vascular homeostasis, it has been shown that the loss of endothelial cells is an outstanding feature of atherosclerosis (refer as well to SERPINB7 section).

In conclusion, the SERPINB1 and members of the SERPINB1 related metabolic pathways, would be able to regulate wide range of cellular processes such as apoptosis, endothelial dysfunction and inflammation and phenotypin traits and conditions such as blood pressure, glucose tolerance/intolerance/type 2 diabetes, ischemic condition in heart (angina pectoris, AMI) or elsewhere, and metabolic syndrome. We present below empirical evidence supporting these associations (Table 2).

The squamous cell carcinoma antigen (SCCA) represents 2 other members of the serpin family, which we assign herein to a group of ov-serpins with a significant relevance in cardiovascular disease and its complications. It has been isolated from superficial and intermediate layers of normal squamous epithelium, whereas its RNA was found in the basal and subbasal levels. It has been firstly isolated from metastatic squamous cell carcinoma and in time has been accepted as a circulating tumor marker for squamous cell carcinoma, with highly predictive potential for recurrent disease (OMIM entry 600517). Interestingly, in the cytoplasm of normal cells has been detected only the neutral form of the SCCA protein, while the acidic form of the protein has been primarily found in malignant cells and in the plasma of cancer patients. Schneider et al, 1995, have presented appealing findings in the 18q21.3 region, identifying a DNA fragment with 98% identity in the exonic regions to the previously found SCCA1 gene, called SCCA2. SCCA1 and SCCA2 have been tandemly arrayed and predicted to encode for the neutral and acidic form of the SCCA respectively (Schneider et al, 1995).

SCCA1 gene covers 24.6 kb on the 18q21.3 region, from 59455473 to 59480107 bp. It encodes for a protein of 390 aminoacids with a molecular weight of 44.6 kDa. Known aliases are SERPINB3, HsT1196, SCCA1, Protein T4-A. It seems that the SCCA1 gene is highly expressed, especially in tongue, lungs, muscle, cervix, Hassal's corpuscules in thymus, skin, pancreas, blood and brain. Its expression has been closely related to the cellular differentiation both in normal and malignant cells. SERPINB3 protein has been localized in cell cytoplasm, but there is evidence that low levels are present in plasma as well.

Functions of SCCA1 have been primarily investigated with respect to its role in cancer screening and development. A function in immune responsiveness as well as in regulation of proteolysis and peptidolysis has been assigned to its description. A 475 bp promoter region has been identified upstream of the transcription start site, and it has been shown to be upregulated in squamous carcinoma cells (Hamada K et al, 2001). Ets and STAT6 transcritpion factors have been shown to influence the activity of the promoter region of both SCCAs (Iwasaki M et al, 2004; Suminami Y et al, 2005).

SCCA1 exhibits potent inhibitory effects on cysteine proteinases-cathepsin K, L and S, and papain (Schick C et al, 1998). As most of the serpins display inhibitory properties on serine proteinases, the SCAA1 member has been acknowledged as a cross-class inhbitor.

Besides being evaluated in relation to their involvement in cancer development, interesting findings reveal association of the SCCAs with different other diseases, such as psoriasis, or a growth-activated diseased skin (Rivas M V et al, 1997; Takeda A et al, 2002), bronchial asthma (Izuhara K, 2003; Ray R et al, 2005) and brain inflammation-the last visualized as alteration of the microglia morphology (Thakker-Varia S et al, 1998).

Barnes R C and Worrall D M, 1995, identified the SCCA2 gene and estimated a 95.3% identity to the SCCA1 nucleotide sequence. It covers 24.6 kb and encodes for a protein with 44.9 kDa weight and 390 amino acids in length. Other aliases for the SCCA2 gene are SERPINB4 and PI11, as well as leupin, the last being assigned to it because of the leucine residue at the P1 position (Barnes R C and Worrall D M, 1995). There are 6 different amino acids at the rsL in SCCA2 when compared with SCCA1 (Barnes R C and Worrall D M, 1995). Thus, a specific inhibitory activity, different from the one of the SCCA1 protein is expected. In analyses of the promoter region of the SCCA2 gene, Sakaguchi et al, 1999, have mapped a putative TATA box element, as well as binding sites for Ets, NF-IL6 sequence and IRE consensus sequence.

Explanation of the impact of the SCCA genes (SERPINB3 and SERPINB4) and their proteins, especially in relation to our findings would most probably involve more than one pathway, taken into account the broadness of the physiological and pathological processes they have been assigned to so far (see the prior art). The transcription factors found to modulate the SCCA genes do have a broad influence on atherosclerotic, inflammatory and metabolic processes, both in sickness and health. First of them, the Ets-1 transcription factor has been shown to participate in the developing of vascular structures in heart, arteries, capillaries, and meninges, as early as in the embryonal stage, further exerting strong impact on both vasculogenesis and endothelial apoptosis in adult. The Ets-1 pathway has been suggested to be involved in pathological vascular changes during hypertensive remodelling (Kuwahara F et al, 2002).

Ets-1 participates in the numbered processes via activating expression of genes, and among those are the SCCA genes. SCCA proteins are found in blood, thus a role in remodelling of vascular wall and endothelial apoptosis, processes tightly related to atherosclerosis and hypertension, is logical for SCCA proteins.

The Signal Transducer and Activator of Transcription 6 (STAT6) exhibits major roles in processes allied to the interleukines (IL)—main participants in inflammatory cascade and the immune response. It-has been shown that IL4 and IL3 induce a phosphorylation of STAT6 (Quelle F W et al, 1995), the last on turn stimulating IL4 receptor gene expression (Kotanides H and Reich N C, 1996).

Activation of STAT6 by interleukins would lead eventually to SCCA1 and SCCA2 expression, thus presenting another pathway of involvement of SCCAs in inflammatory disease, such as atherosclerosis.

Yet another mechanism could be a pathway comprising SCCAs, STAT6 and leptin. The STAT6 has been shown to be involved in the action of leptin and therefore called a “fat STAT”, together with other members of its family (Darnell J E Jr, 1996). Leptin has pro-inflammatory, proliferative and calcification promoting effects in the vasculature and may be involved in coagulation cascade and fibrinolysis, also leptin has been considered to play a key role in the elevation of sympathetic activity commonly found in obese or/and hypertensive patients (Sharma A M and Chetty V T, 2005).

SCCA1 may act in the atherosclerotic pathway via inhibition of the cathepsins K, L and S, which on turn are essential participants in the development of atherosclerosis, atherosclerotic plaques and their complications (Sukhova G K et al, 1998; Li W et al, 2001). Cathepsins K and L are also inhibited by SERPINB13 as well, thus the samemechanisms are relevant to PI13. Although SCCA2 inhibits catS, as well as other papain-like cysteine proteinases, it acts in a 50-fold lower rate, as compared to the SCCA1 (Luke C et al, 2000). Identified substrates for SCCA2 inhibitory activity are serine proteinases as chymotripsin-like proteinases, cathepsin G and mast cell chymase (Schick C et al, 1997). Same report shows evidence that SCCA2 is a substrate for the action of trypsin, cathepsin S, human neutrophyl elastase and chymotrypsin.

Cathepsin G is a serine proteinase, especially synthesised in neutrophils and mast cells, which substrates include compartments of extracellular matrix (ECM) as laminin, type IV collagen, fibronectin, elastin, proteoglycans (see the part for ECM relevance on atherosclerosis in the SPOCK text), as well as immunoglobulins, complement components, clotting factors and cytokines. It activates platelets, lymphocytes and macrophages. Therefore, a multiple additional pathways involving SCCA2 give further support to our invention.

Blood pressure is regulated among other things by the renin-angiotensin-aldosterone system. The conversion of either angiotensinogen or angiotensin I to the vasoactive polypeptide angiotensin II is regulated by cathepsin G (Schick C et al, 1997). As cathepsin G is a substrate for SERPINB4, it presents another pathway for our invention.

Since the SERPINB4 was shown to inhibit the mast cell chymase a detailed description on the consequent impact on cardiovascular disease has been given in the section of SERPINB11 and SPINK5L3 genes.

Sakata Y et al, 2004, demonstrated that SCCA2, but not SCCA1, inhibited the cysteine proteinase activity of group 1 mite allergens (Der p 1 and Der f 1). SCCA2 contributed the suicide substrate-like mechanism without formation of a covalent complex, causing irreversible impairment of the catalytic activity of Der p 1. Der p 1 and Der f 1 are allergens closely correlated to bronchial asthma, atopic dermatitis and allergic rhinitis. As Der p 1 may activate the secretion of inflammatory cytokines and the induction of the Th2 subset of T lymhpocites, it may initialize a pathological inflammatory cascade providing evidence that SERPINB4 may regulate the inflammatory cascade. Furthermore, it has been shown that SCCA2 inhibits the TNFα-apoptosis and caspase-3 activation (McGettrick A F et al, 2001), giving extra proof of the anti-inflammatory/antiapoptotic function of the SERPINB4 protein and suggesting a role in asthma/allergy and cardiovascular diseases.

We present empirical evidence linking the SERPINB3 and SERPINB4 genes with CHD, AMI, HT, stroke, MBO and obesity (Tables 2, 3, 20 and 22 and text).

SERPINB5 (alias maspin) locates in 18q21.3 SERPINB gene cluster region (OMIM). Maspin was originally found from human corneal cells, endothelium, and stroma, and was hypothesized to function within the cornea to regulate cell adhesion to extracellular matrix molecules, and perhaps to regulate the migration of activated fibroblasts during corneal stromal wound healing. Maspin is an exceptional ov-serpin as it has not been shown to inhibit serine or cysteine proteinases. In contrast, maspin specifically inhibits prostate cancer-associated urinary plasminogen activator and prostate cancer cell growth, and inhibits the growth of vascular smooth muscle cells. In tumours, it also decreases angiogenesis. Protease inhibition seem not to account maspins' activity as a tumor suppressor. Maspin expression is dependent on cytosine methylation of the maspin gene promoter. Methylation controls, in part, normal cell type-specific maspin expression. The maspins role in vascular smooth muscle cells and in angiogenesis makes it a potential candidate gene in cardiovascular and metabolic disorders.

The serine proteinase inhibitor, member 7 (SERPINB7), or otherwise called megsin (after mesangial cell-specific gene with a homology to serpin), is another representative of the ov-serpin cluster, with significant implication on cardiovascular risk. Megsin has been found significantly expressed in kidney, predominantly in mesangial cells, and specifically in relation to mesangioproliferative glomerulonephritis and diabetic nephropathy. Besides kidney SERPINB7 is expressed in pancreas, liver, lung, placenta, skin, tongue and heart (Miyata T et al, 2002). SERPINB7 gene maps next to other serpin genes, on the 18q21.33 cytogenetic band, expands on 52.3 kb and encodes for a protein of 380 amino acids and molecular mass of 42.9 kDa. The megsin gene and protein possesses the specific characteristics of the other serpins such as uncharged, small and nonpolar residues at the rsL, ATAA sequence at the NH₂ region, the so-called hinge region, and a preserved β-sheet stretch (significant for the inhibitory activity of serpins) (Miyata T et al, 1998).

A recent study on the promoter region of the Megsin gene revealed a cis-acting element, possessing highly conserved binding motifs such as AP-1, Oct-1 and TCF11 (Inagi R et al, 2002). After detailed analyses the AP-1 binding motif has proved to be a good candidate for a transcription factor of megsin gene (Inagi R et al, 2002). AP-1 regulates variety of genes and protein activities, involved in immune, inflammatory, and growth control processes, and having impact on cell adhesion, differentiation and matrix formation.

As mentioned above, SERPINB7 has been related to a pathological condition, described as IgA nephropathy. IgA nephropathy is a very common pattern of glomerulonephritis, eventually progressing to renal insufficiency. Several studies have shown that IgA nephropathy is a consequence of host susceptibility, rather than an intrinsic kidney abnormality (Barratt J et al, 2004). Three main characteristics of IgA nephropathy are 1) increased production of IgA which favours mesangial deposition, 2) “responsiveness” of the glomerular mesangium expressed as susceptibility to IgA deposition, capacity to mount the inflammatory response and capacity to prevent a future glomerular sclerosis, and 3) tendency of whole kidney to respond to the damage by mounting a response which favours progressive renal injury (hypertension, proteinuria, tubular atrophy, intersticial fibrosis) (Barratt J et al, 2004). Transgenic megsin mice model has provided evidence for altered metabolism of components of cellular matrix in kidney, with marked accumulation of type IV collagen and laminin in the sclerotic glomeruli (Miyata T et al, 2002).

There may be few mechanisms by which SERPINB7 is related to blood pressure and atherosclerosis. An obvious and incorporated outcome of IgA glomerulonephritis is elevated blood pressure. On the other hand elevated blood pressure by itself leads to alteration of glomerular vessels, as far as to glomerular sclerosis.

We suggest that like other serpin proteins megsin probably acts as a proteinase inhibitor; however its may have other functions as well. It has been shown that some of the serpins function as ligands. It is possible that SERPIN7 promotes inflammation in tissues and organs where it is expressed by being a ligand e.g. for IgA antibodies, certain antigens (Ag) and/or autoimmune complexes (Ab/Ag). Atherosclerosis is an inflammatory disease and it has been shown that antibodies against oxidised LDL particles (IgA included) significantly influence progression of diseases such as atherosclerosis, diabetes, renovascular syndrome, uremia, rheumatic fever, morbus Bechtjerev and lupus erythematodes (Steinerova A et al, 2001). As SERPINB7 is present is vessel walls, complexes between SERPINB7 and IgA or IgA/Ag complexes could promote coagulation and/or inflammatory cascade, as well as binding of pro-atherogenic cells (Mf) or complexes with consequent development of atherosclerosis.

Mesangial IgA deposition seems to be widespread in individuals with no clinical evidence of renal disease at the time when there is already evidence of mesangial IgA (Barratt J et al, 2004). The juxtaglomerular apparatus, whose myoepithelial cells are responsible for the renin secretion is situated at the vascular pole of glomerulus. Thus, even a slight alteration of the glomerulus structure (because of traces of antibody or antibody/antigen complexes) can have a significant impact on the structures nearby, in this case the juxtaglomerular apparatus, which is involved in renin secretion and consequently the regulation of blood pressure.

According to our invention, SERPINB7 could act as a protease inhibitor and modify the activity of several proteases of the serpin families involved in broad pathways such as coagulation, complement activation, inflammation and immune response. SERPINB7 may also act as a ligand for components of extracellular matrix. Both functions may finally result e.g. to extracellular matrix remodelling and affect consequently function of organs (e.g. heart and vessels).

We present empirical support for the role of SERPINB7 in CHD, AMI, HT, stroke, MBO and obesity in Tables 2, 3, 20 and 22 and in the text of the experimental section. SERPINB2 (alias Plasminogen activator inhibitor-2; PAI; PAI2; PAI-2; PLANH2; HsT1201) is a specific inhibitor of plasminogen activators (OMIM). PAI2 is also known as monocyte arg-serpin. PAI2 is located in chromosome 18q21.2-q22 SERPINB gene cluster with opposite transcriptional orientation. Although a large body of information has accumulated on the biology, biochemistry, and clinical aspects of PAI2, suggesting that it is involved in many physiologic and pathologic processes, its precise role in placenta, in pregnancy plasma, in skin, and in inflammatory conditions, as well as the diagnostic and therapeutic possibilities of PAI2, remain to be established. PAI2 is thought to serve as a primary regulator of plasminogen activation in the extravascular compartment. High levels of PAI2 are found in keratinocytes, monocytes, and the human trophoblast, the latter suggesting a role in placental maintenance or in embryo development. The primarily intracellular distribution of PAI2 may also indicate a unique regulatory role in a protease-dependent cellular process such as apoptosis. This evidence suggest a potential role for PAI2 in cardiovascular and metabolic diseases.

Our HPM haplotype region analyses showed that a region between the SERPINB7 and SERPINB2 genes was associated with AMI and hypertension, which suggests a role of these genes with the studied traits.

SERPINB8 (alias cytoplasmic antiproteinase 2; PI8; CAP2) is a cytoplasmic serine-protease inhibitor expressed as two transcripts of 1.4 and 3.8 kb (OMIM). The 1.4-kb transcript is most abundant in liver and lung while the 3.8-kb transcript is most abundant in skeletal muscle and heart. Recently, PI8 has been found expressed in the epithelium of oral cavities, skin, monocytes, and by neuroendocrine cells in the pituitary gland, pancreas, and digestive tract. PI8 localizes in monocytes in the nucleus, whereas neuroendocrine cells have only cytoplasmic form. Thus, PI8 may have many different currently uncharacterised functions. SERPINB8 contains dual reactive sites and it uses more than one active P1 residue for inhibitory activities. These properties support a potential role of SERPINB8 in cardiovascular and metabolic disorders.

In multivariate analysis, SNP marker rs213069 residing between the SERPINB8 and C18orf20 genes was associated with plasma insulin. Furthermore, SNP markers rs4940605 and rs8094641 downstream from SERPINB8 gene were associated with metabolic syndrome and the SNP rs4940605 was associated also with HDL cholesterol levels.

The Serine proteinase inhibitor member 9, known as well as proteinase inhibitor 9 (PI9), cytoplasmic antiprotease 3 (CAP-3, CAP3) and SERPINB9 belongs to the serpin cluster situated on the 6^{th} chromosome. The gene of SERPINB9 covers 16 kb from the 6p25.2 cytogenetic band and encodes for a 376 amino acid protein with molecular mass of 42.4 kDa. The PI9 protein is characterised by high degree of similarity with the amino acid sequences of other members of the ov-serpins, such as EI, PAI-2 and SCCA (Sprecher C A et al, 1995). PI9 possesses all the structural characteristics of the other ov-family members. However SERPINB9 possesses unique acidic P₁ (Glu³⁴⁰-Cys³⁴¹) residue at the reactive center (Sprecher C A et al, 1995) and contains a consensus site for attachment of N-linked carbohydrates. Glycosylation of SERPINB9 presents an opportunity for the SERPINB9 to be secreted to extracellular space despite of its intracellular protein characteristics (Sprecher C A et al, 1995).

SERPINB9 has been detected in many tissues, especially in lymphoid organs, and interestingly in endothelial and mesothelial cells, which might stand for its function as a protector of misdirected apoptosis (Buzza M S et al, 2001). Specific sites of expression are immunologically active organs such as eye lens, ovary, testis and placenta (OMIM: 601799). PI9 is expressed by activated mast cells suggesting a protection against apoptosis and cell-destruction during an inflammatory response in these cells (Bladergroen B A et al, 2005). High expression of SERPINB9 has been detected in the tubular epithelial cells of renal allografts (Rowshani A T et al, 2004), which might imply for another self-defensive mechanism against probable rejection (defined by apoptosis). This potentially protective mechanism—expression of SERPINB9—against the action of cytotoxic lymphocytes and their proteases has been “utilized” by several types of cancer cells, e.g. by non-Hodgkin and Hodgkin lymphomas (Bladergroen B A et al, 2002) and pediatric acute lymphoblastic leukemias (Classen C F et al, 2004).

PI9 is a potent inhibitor of Granzyme B-a main protease of cytotoxic T lymphocytes and natural killer cells (NK) (Krieg S A et al, 2001). Another substrate inhibited by PI9 is the caspase-1, or the so-called interleukine (IL) 1β-converting enzyme (Sun J et al, 1996). Caspase-1 has been shown to regulate key steps in inflammation and immunological reactions, by activating proinflammatory cytokines or mediating apoptosis (Young J L et al, 2000). As the IL1β itself strongly induces PI9 there seems to be a negative feedback loop controlling the PI9 function (Kannan-Thulasiraman P and Shapiro D J, 2002). Regulation of activity of enzymes like caspase-1 and Granzyme B, and the induction by cytokines such as TNFα and interferone γ (IFNγ) indicate participation of the SERPINB9 in inflammation and apoptosis, which are tightly related to atherosclerosis.

Additional data indicates that SERPINB9 has other targets of inhibition, such as the bacterial subtilisin A (Kannan-Thulasiraman P and Shapiro D J, 2002.) and the neutrophil elastase (Dahlen J R et al, 1997). Furthermore, besides IL1β, antiviral cytokines such as TNFα and interferone γ (IFNγ) induce the expression of PI9 (Dahlen J R et al, 1999). While PI9 inhibits Granzyme B, another representative of the granzyme family, and specifically the Granzyme M- a serine protease involved in the induction of death of target cells by cytotoxic lymphocytes, has been shown to inactivate PI9 (Barrie M B et al, 2004). Estrogen has been observed to promote PI9 espression via a unique estrogen responsive element present downstream from the PI9 transcription site (Mahrus S et al, 2004). PI9 is expressed broadly in tissues and systems, via its presense in the immune system cells. It is particularly expressed in the endothelial cells (Buzza M S et al, 2001). Endothelium as a single-cell lining internal cover of vessels has important function in regulation of e.g. vascular tone and permeability, platelet adhesion and aggregation, activity of the coagulation system. Endothelial dysfunction has been related to conditions, such as hypertension, diabetes, hypercholesterolemia and others, all related to cardiovascular risk and atherosclerosis. SERPINB9 has been suggested to protect endothelial cells from uncontrolled apoptosis (Buzza M S et al, 2001), and thus SERPINB9 could regulate functions of endothelial cells.

SERPINB9 is clearly associated with immune response control, inflammation and apoptotic processes as PI9 has been shown to inhibit several proteinases, involved in broad pathological cascades, including atherosclerosis, inflammatory and apoptotic disease. In addition SERPINB9 may inhibit other serpin proteases, thus having a role in preserving the functional integrity of the endothelium.

Empirical support for the role of SERPPNB9 in CHD, AMI, HT, stroke, MBO and obesity and related quantitative traits is presented in Tables 2, 3, 20 and 22 and in the text of the experimental section below.

SERPINB12 (alias yukopin) is a newly identified, poorly characterized member of ov-serpins located in chromosome 18 SERPINB gene cluster. SERPINB12 inhibits trypsin and plasmin, but not thrombin, coagulation factor Xa, or urokinase-type plasminogen activator. SERPINB12 is expressed in many tissues, including brain, bone marrow, lymph node, heart, lung, liver, pancreas, testis, ovary, and intestines. Broad expression pattern suggests widely dispersed functions for the protein. Presence in tissues affected by cardiovascular and metabolic diseases suggests a potential role for SERPINB12 in these diseases.

According to this invention SERPINB12 gene surrounded by the SNP markers rs10503083 rs1455564 and rs10503083 is associated with hypertension in the HPM haplotype region analysis (Table 2). In addition, a SNPmarker rsl455564 residing between the SERPINB5 and SERPINB12 genes, clearly associates in this study with prevalent CHD, and evidence thus indicates a role for SERPINB5 and SERPINB12 in cardiovascular and metabolic diseases.

SERPINB13 is known as HaCaT UV-repressible serpin (hurpin), the protease inhibitor 13 (PI13), HUR7 and headpin. It maps next to the other serpins identified at the 18^{th} chromosome, at the 18q21.33 cytogenetic band. The SERPINB13 gene covers 11.8 kb and encodes for a protein with 391 amino acids and 44.3 kDa molecular weight. As for the other serpins a cellular localisation has been suggested also to headpin. Expression studies denote so far larynx, lung, muscle, skin, tongue and uterus as possible sources for PI13. Additionally, Moussali et al, 2005, have detected hurpin in endothelial cells.

Hurpin has been expressed in relation to the proliferation state of keratinocytes (Abts H F et al, 1999), which may imply for a role in the cell proliferation and/or differentiation. Interestingly it has been overexpressed in psoriatic lesions, which are generally characterised by hyperproliferation and therapeutic responsiveness to UV-radiation (Abts H F et al, 1999). SERPINB13 has been downregulated in certain squamous cell carcinomas (Nakashima T et al, 2000) and activated in others (Moussali et al, 2005).

The putative reactive center of hurpin has been characterised as Thr356-Ser357 (Abts H F et al, 1999). By similarity with the other members of the seprin family, SERPINB13 is expected to have inhibitory activity against a number of cysteine and serine proteinases. Jayakumar et al, 2003, have demonstrated that PI13 is a potent inhibitor for cathepsins K and L.

It is eveident that members of the human serpins regulate a diverse array of serine and cysteine proteinases associated with essential biological processes such as fibrinolysis, coagulation, inflammation, cell mobility, cellular differentiation, and apoptosis (Askew et al, 2001). In addition, several serpins perform also other functions such as hormone transport (thyroid-binding globulin (SERPINA6), corticosteroid-binding globulin (SERPINA7)), and blood pressure regulation (angiotensinogen (SERPINA8)) (Silverman GA et al, 2001, Scott FL et al, 1999).

Thus serpins are involved in a diverse set of biologic functions that extend beyond the ability of these molecules to irreversibly inhibit target proteinases. A protein binding function of serpins cannot be ruled out in regulation of broad pathological and physiological processes such as activation of apoptosis, inflammation, coagulation, complement activation and formation of atherosclerotic plaques. As each serpin discussed herein is characterised by multiple functions in multiple physicochemical processes serpins may link different traits and phenotypes, e.g. atherosclerosis (and its complications) and allergy (atopic diseases), atherosclerosis (and its complications) and different types of cancers, and atherosclerosis (and its complications) and skin diseases (psoriasis, atopic dermatitis, etc.).

Kazal Type 5 Serine Peptidase Inhibitors SPINK5, SPINK5L2 and SPINK5L3

The presence of Kazal motifs is characteristic for Kazal type peptidase inhibitor family I1, clan IA (Rawlings ND et al, 2004). Kazal inhibitors contain 1 to 15 Kazal-type inhibitor units and the inhibitor motif makes 11 contacts with its enzyme substrate: unusually, 8 of these important aminoacid residues are hypervariable (Laskowski M et al, 1987). Alterations in the enzyme-contact residues, especially those forming the active site bonds, affect the strength and specificity of inhibition of particular serine proteases (Williamson M P et al, 1984; Empie M W, Laskowski M. 1982). The Kazal domains often occur in tandem arrays and have a small alpha+beta fold containing three disulphide bridges. Kazal inhibitors exert reversible inhibitor activity towards serine peptidases of the S1 family such as trypsin and elastase (Mulder N J et al, 2005) and they form a subset of proteins including pancreatic secretory trypsin inhibitor, avian ovomucoid, acrosin inhibitor and elastase inhibitor. Kazal-type peptidase inhibitor motifs are detectable in the SPINK5, SPINK5L2 and SPINK5L3 proteins.

Kazal-like domains are also seen in the extracellular part of agrins which are not known to be proteinase inhibitors. Agrin is a large multidomain heparin sulphate proteoglycan (Tsen G et al, 1995), discovered as a synapse-organizing molecule at the neuromuscular junction. It is highly concentrated in the synaptic basal lamina, and is known to induce aggregation of acetylcholine receptors on the myotube surface as well as to be necessary for the differentiation of the presynaptic apparatus. Agrin may as well play a role in the basement membrane of the microvasculature and in the synaptic plasticity (Donahue J E et al, 1999).

SPlNK5 gene is the most studied from the three SPINK genes to be discussed herein. SPINK5 (LEKTI, LETKI, VAKTI, NETH) covers 73.28 kb. It encodes for a protein with 1064 amino acids and a molecular weight of 12.1 kDa. It encodes for a multidomain serine protease inhibitor consisting of a signal peptide preceding 15 potential Kazal-like domains, having characteristics of serine peptidase inhibitors, among which at least one has antitrypsin activity. From all domains only two (D2 and D15) perfectly match the typical Kazal motif, while the other 13 represent a Kazal-type derived four-cysteine residue pattern (Bitoun E et al, 2003). A tropomyosin domain, found in the SPINK5 structure might imply for an extra function of SPINK5. According to InterPro database tropomyosins, are a family of closely related proteins present in muscle and non-muscle cells. In striated muscle, tropomyosin mediates the interactions between the troponin complex and actin so as to regulate muscle contraction. The role of tropomyosin in smooth muscle and non-muscle tissues is not clear. However, some of the proteins in this family are known to have a role as allergens.

It has been suggested that LEKTI is a probable precursor protein, inactive before a proteolytic processing of subtilisin-like proprotein convertases, and more precisely furin (Bitoun E et al, 2003). Deletion of the N-terminal signal peptide of LEKTI has been associated with altered distribution, reduced stability and failure to become secreted (Jayakumar A et al, 2005). Experiments with deletion of the C-terminal domain have shown unaltered stability but disturbed secretion (Jayakumar A et al, 2005). The SPINK5 protein is expressed broadly, e.g. in epithelial and mucose surfaces, brain, cervix, colon, heart, kidney, muscle, larynx, lung, skin, placenta and stomach, both in adult and embryonic tissues. Besides belonging to the I1 inhibitors family, SPINK5 belongs to a two-member protein family, another member being SPINK6 precursor protein. Publicly available databases (Psort and LocusLink) suggest both intra- (cytoplasmic and mitochondrial) and extracellular localisation of the SPINK5 protein. The different localisations may well apply to the different isoforms of the protein and consequently to different functions.

SPINK5 has been the first serine-protease inhibitor shown to be involved in a skin disorder, namely the Netherton syndrome (NS). NS is a severe autosomal recessive disorder characterised by congenital ichthyosiform generalized erythroderma, a specific hair defect (trychorrhexis invaginata, “bamboo hair”) and a broad range of allergic manifestations including atopic dermatitis and elevated IgE level (OMIM: 256500). A common feature of NS is the deficiency of SPINK5 expression in the epidermis accompanied by increased serine protesase activity in stratum corneum resulting in epidermal detachment, desmosomal dissociation and destabilization of corneodesmosin. Thus main functions of SPINK5 may be regulation of terminal epidermal differentiation and comeocyte desquamation, as well as hair growth and differentiation.

The recombinant SPINK5 protein inhibits trypsin, plasmin, subtilisin A, cathepsin G and elastase (Mitsudo K et al, 2003). Putative substrates for SPINK5 peptidase inhibitory activity are proteases and proteinases expressed in same tissues as SPINK5, examples of such are membrane-type serine protease 1, probably involved in keratinocyte differentiation, the trypsin-like mast cell tryptase- a major mediator of inflammatory and allergic conditions, and the trypsin-like kallikrein 6 serine protease, expressed in the Hassal corpuscles. Thus SPINK5 could well be involved in numerous biological pathways, related to inflammation, anti-microbial defence and immunological response.

The serine septidase inhibitor kazal type 5-like 2 (SPINK5L2) gene is another member of the SPINK5 gene cluster described herein. The gene covers 5.67 kb at 5q32. It encodes a protein of 97 aminoacids, molecular weight of 11057 Da, with one Kazal peptidase inhibitor domain. The SPINK5L2 gene has three introns and four exons and it seems that the SPINK5L2 protein is most likely secreted. The biological role of the SPINK5L2 protein is unclear, but it is expected to be involved in the protease/proteinase inhibition, due to the presence of the Kazal inhibitor domain and its structural similarity with the SPINK5 gene. In addition SPINK5L2 protein may have functions comparable to the ones of the agrin (see above).

The third member of SPINK5 gene cluster is serine protease inhibitor kazal type 5-like 3 (SPINK5L3) encoding SPINK5L2 like protein. SPINK5L3 maps to the 5th chromosome, at the 5q32 cytogenetic region and covers 18.07 kb. PSORT II analyses suggest that the SPINK5L3 protein contains one Kazal peptidase inhibitor domain and that the it is extracellular protein. SPINK5L3 gene is expressed at least in kidney, eyes, testis, blood cells and skeletal muscle.

A number of studies have shown association of SPINK5 gene polymorphisms with atopic dermatitis (Walley A J et al, 2001; Kato A et al, 2003), and the study of Kabesch M et al, 2004, showed the association of SPINK5 gene with asthma in German population. All this with the SPINK5 associated atopic manifestations in NS suggests that SPINK5 is a downregulator of allergy related immune responses. Moreover, the high expression of SPNK5 in thymic Hassall's corpuscles suggests its regulatory impact on the T-cell maturation, tolerization and activation, and in our view, an impact on immune and inflammatory processes playing key role in atherosclerosis.

We present empirical evidence supporting the role of SPINK genes in CHD, AMI, HT, stroke, MBO and obesity (Tables 7-12, 19-22). Several SNP markers either in the SPINK genes or in the SPINK gene region were associated with the risk of CHD, AMI, HT, stroke, MBO and/or obesity as well as with several traits related to these conditions such as coagulation factors, acute phase proteins, plasma lipids and lipoproteins.

Sparc/Osteonectin, cwcv and Kazal-Like Domains Proteoglycans (SPOCK1, SPOCK2 and SPOCK 3)

SPOCK1 gene (aliases are TESTICAN, TIC1 and SPOCK) is located in chromosome 5 at 5q31 and the gene covers 137.28 kb on the reverse strand and encodes sparc/osteonectin, cwcv and kazal-like domains proteoglycan (Charbonnier F et al, 1998). The protein product of SPOCK1 has 439 amino acids and molecular weight of 49.124 kDa. SPOCK1 was first identified in human seminal plasma, consequently named as testican, and has since been identified in several other tissues (Alliel P M et al, 1993; Bonnet F et al, 1992). The mRNA levels are highest in brain, prostate, heart, testes, placenta, uterus and kidney (BaSalamah M A et al, 2001). Within tissues, testican is generally expressed in endothelial cells (Marr H S et al, 1997). Embryonic expression of testican is strong in the nervous system during neuronal migration and axonal growth period (Charbonnier F et al, 2000), and is present in neurons (Marr H S et al, 2000) and in mature synapses (Bonnet F et al, 1996) suggesting that testican might function in neuronal development and in the maturation of synaptic connections. In human brain, testican is also expressed in vascular epithelial cells, and in astrocytes in areas of reactive gliosis resulting from infarction (Marr H S et al, 2000), indicating a potential role for testican in ischemic conditions. Furthermore, in mice testican mRNA co-localizes to neuromuscular junctions with acetylcholine receptors (Cifuentes-Diaz C et al, 2000), one of the major cardiac rhythm regulating neurotransmitter receptors, which might indicate an important role for testican in innervation of heart.

Glycolysated high molecular weight form (130 kDa) of SPOCK1 has been detected in blood and SPOCK1 fragments of 35, 36 and 38 kDa in blood (BaSalamah M A et al, 2001), cerebral spinal fluid (Stark M et al., 2001), and in human semen (Bonnet F et al, 1992). Fragmentation of the full-length SPOCK1 appears to occur at the thyroglobulin-like domain region by serine-proteases, and therefore it has been proposed that testican's putative cysteine protease inhibitor activity may itself be degraded by proteases in plasma (BaSalamah M A et al, 2001). Fragmentation to smaller forms may also be related to protein decay process in the blood (BaSalamah M A et al, 2001).

SPOCK1 is synthesized as a precursor protein containing signal sequence, and is secreted as a proteoglycan-like molecule to the extracellular matrix (ECM). ECM regulates the behaviour of the contacting cells by influencing cell development, migration, proliferation, shape and function. Proteoglycans in ECM form an aqueous substance permitting diffusion of small molecules between blood and tissue cells and regulate the activities of other types of secreted proteins such as proteases and protease inhibitors. Testican has partial homologies to other secreted proteoglycans such as insulin-like growth factor binding proteins, thyropins, agrin and nidogen which are potentially involved in cellular responses via receptor-mediated signalling or by inhibition of enzymes involved in tissue remodelling (BaSalamah M A et al, 2001).

Testican has several protease inhibitor-like domains and is assigned as a compound inhibitor according to MEROPS classification (Rawlings N D et al., 2004). Three structurally and finctionally distinct motifs are observed: 1) Kazal type serine protease inhibitor domain characteristic to MEROPS inhibitor family I1, clan IA members, which inhibit serine peptidases of the S1 family, and follistatin (FS) domain often found in serine-protease inhibitors and follistatin family members playing an important role in tissue specific regulation; 2) extracellular calcium-binding domain (SPARC_EC) found in cell-matrix interaction regulating protein SPARC/osteonectin and related proteins (QR1, SC1/hevin, and tsc-36/FRP). The extracellular domain interacts with follistatin domains to stabilize calcium binding, which in turn is proposed to promote symmetric homodimerization of EC-FS modules; and 3) thyroglobulin-type repeats domain characteristic to MEROPS proteinase inhibitor family I31, clan IX type I TY-repeats which are proposed to function in cysteine protease inhibition. In addition, the acidic C-terminal region has binding sites for glycosaminoglycans chondroitin and heparan sulfate. Testican is classified according to MEROPS database as an inhibitor (inhibitor I31.006) not belonging to any clan but as a member of inhibitor Family I31.

Possible substrates for Kazal-domain containing proteinase inhibitors and consequently following presumed function have been presented above in conjunction with SERPINB11 and SPINK5L3. Interestingly, Kazal-like domain found in testican is more homologous to the Kazal-domain found in osteonectin/SPARC gene than to Kazal-like domains of any other protease inhibitors (Charbonnier F et al, 1998; Bocock J P et al, 2003). Osteonectin/SPARC lacks protease activity, but is a potentially anti-adhesive and angiogenesis promoting factor which regulates the activity of growth factors, such as platelet-derived growth factor, fibroblast growth factor 2, and vascular endothelial growth factor (Motamed K and Sage E H, 1997; Brekken R A and Sage E H, 2000). SPARC deficiency has been associated with increased adiposity in mice (Bradshaw A D et al, 2003), a significant trait in metabolic disorders. Thus, in our view also SPOCK1due to protein homology could play a role in fat deposition in ECM, and might thereby affect lipid metabolism.

Thyroglobulin type I repeat domain(s) containing proteins belong to MEROPS equistatin proteinase inhibitor family I31, clan IX. Other members of this small family include finctionally diverse proteins such as MHC II invariant chain p41 form, thyroglobulin, and insulin-like growth factor binding proteins. I31 family proteinase inhibitors typically bind reversibly and tightly to cysteine peptidases of family C1. Peptidase family C1 contains both endo- and exopeptidases, and is classified into two subfamilies, C1A (secreted and lysosomal enzymes) and C1B (soluble intracellular aminopeptidases). Furthermore, the C1 memebers are recognized candidate drug targets, as cathepsin B for cancer, cathepsin S for control of antigen presentation and attenuation of immune response, cathepsin K for the control of osteoporosis, and cathepsin L for antigen processing and tumour cell metabolism. SPOCK1 has been shown to inhibit cathepsin L, a ubiquitously expressed protease normally enriched in lysosomes (Bocock J P et al, 2003). This inhibition is independent of the glycosaminoglycan chains of testican, and provides evidence that lysosomal protease activity is in the protein backbone of the testican (Bocock J P et al, 2003).

C1 family of peptidases includes also proteins classified as non-peptidase homologs, which are putative peptidases that either lack experimental peptidase activity or lack one or more of the conserved active-site residues essential for activity. There are another possible target protease for SPOCK1 in the C1 family, the tubulointerstitial nephritis antigen-related protein (TIN-ag-RP, gene alias LCN7/lipocalin 7/ARG1/TINAGRP, protein name P3ECSL). LCN7 is a putatively enzymatically inactive papain subfamily (C1A) peptidase expressed predominantly in vascular smooth muscle cells, but also in cardiac and skeletal muscle cells and in kidney (Wex T et al., 2001). It is secreted to extracellular matrix via endosomal trafficking pathway, but the function of LCN7 in unclear (Wex T et al., 2001). Nevertheless, LCN7 and SPOCK1 are expressed in tissues affected by cardiovascular diseases, so they could form complexes and have a role in cardiovascular diseases.

SPOCK1 may mediate its actions by any of its known domains, most of which are associated with protease inhibition. Considering strong expression of testican in heart and in neurons together with ECM binding properties suggests a role for testican in the regulation or modification of innervation of cardiac tissue. Modification of innervation could take place by regulating neuronal growth and synapse maturation to cardiac muscle during development or during cardiac muscle remodelling following ischemia and/or impaired ECM attachment or altered diffusion properties in the affected tissues. Furthermore, modification of blood coagulation and angiogenesis by inhibiting S1 family serine peptidases, as well as C1 family cysteine proteases would be another explanation to high SPOCK1 expression in heart and neurons.

Observation that purified testican and testican-2 inhibit cell attachment and neurite outgrowth in cell cultures supports the postulated SPOCK1 function as a component or a modifying factor of ECM (Marr H S and Edgell C J, 2003; Schnepp A et al, 2005). Furthermore, N-terminal regions of testicans 1 and 3 have been shown to interfere with activation of matrix metalloproteinase 2 (MMP-2, gelatinase A) degrading type IV collagen, a major structural component of basement membranes (Nakada M et al, 2001). Involvement of SPOCK1 in the regulation of inflammation in ECM is also possible as it seem to participate in cartilage turnover (Hausser H J et al, 2004) and differentiation of macrophages into foam cells during atherosclerotic process (Shiffinan D et al, 2003). SPOCK2 (alias testican-2) is located in chromosome 10q. Testican-2, like SPOCK1 is a secreted protein potentially localized in extracellular matrix. This calcium-binding protein, like other members of the family, has been suggested to participate in diverse steps in neurogenesis.

SPOCK3 (sparc/osteonectin, CWCV, and Kazal-like domain proteoglycan 3 alias TES-3, testican-3) is located at chromosome 4q32.3. The characteristics of the gene and the protein encoded by it are similar to that of SPOCK; the length of the 11-exon gene is 501,167 bp, and it encodes for a 436 amino acid precursor protein of molecular weight of 49.071 kDa. Domain structure of SPOCK3 is similar to SPOCK1 and it is composed of a putative signal sequence, unique testican family domain, a Kazal and follistatin domain, extracellular calcium-binding SPARC-EC domain, and a thyroglobulin-type repeats domain, followed by a COOH-terminal domain with two putative glycosaminoglycan binding sites (Nakada M et al, 2001). Like testican, testican 3 is a secreted calcium binding glycoprotein located in the extracellular matrix. The full-length protein shares 51% and 44% amino acid homology with testican 1 and testican 2 proteins, respectively (Nakada M et al, 2001).

Three different isoforms of SPOCK3 have been identified, each originally derived from a different tissue source (brain, fetal kidney, lung/muscle). N-Tes is a well-characterized truncated isoform of SPOCK3 without the thyroglobulin domain and the COOH-terminal region with putative glycosaminoglycan binding sites (Nakada M et al, 2001). N-Tes, like full-length testican 3, is strongly expressed in the brain, and the intensity of expression in the brain is more than that of in the kidney (Nakada M et al, 2001). Both proteins are soluble whereas only the full-length testican 3 has been identified in the ECM in culture (Nakada M et al. 2001).

SPOCK3 is classified in MEROPS database to family I31 unassigned peptidase inhibitor homologues, a class that includes a mixture of proteins that are homologous to known inhibitors, but that are not necessarily inhibitors themselves, or the inhibitor status is unknown. Despite the multiple protease inhibitor domains, testican 3 has not yet been shown to inhibit serine or cysteine proteases. However, both testican 3 and N-Tes, like testican 1, inhibit the activation of MMP-2 by MT1-MMP and MT3-MMP (Nakada M et al, 2001). Furthermore, the expression of both proteins is down-regulated in gliomas indicating a protective role of these proteins in the maintenance of the ECM integrity (Nakada M et al, 2001). In fact, a N-Tes variant has been suggested to have therapeutic potential for gliomas (Nakada M et al. 2001). Testican 2 (SPOCK2) has been shown to abolish the inhibition of MT1- and MT3-MMPs by testicans 1 and 3 (Nakada M et al, 2003). This indicates that SPOCK2 could regulate the function and activity of SPOCK1 and SPOCK3 for example in neurons where thet all are expressed.

The minor allele C of the SNP marker rs4976445 in intron 1 of the SPOCK1 gene is association with inceased risk of CHD-related outcomes, especially with incidence of AMI and family history of CHD in our study population. The SNP marker correlates with cardiovascular and metabolic risk factors, such as increased diastolic blood pressure, increased serum apolipoprotein B level, and low serum HDL-to-LDL cholesterol ratio. The intron 1 of SPOCK1 is very long (231.316 bp) and the SNP marker rs4976445 is located close to highly conserved region. There seem to be an open reading frame −200 to +20 bp from the location of rs4976445 suggesting the presence of alteratively spliced exons in this region (Charbonnier F et al, 1998). Thus, the SNP marker rs4976445 may be related to alternative splicing of SPOCK1 transcripts.

Ischemic events (angina pectoris, AMI) can be a result of arrhythmic occurrences in cardiac muscle. Considering the high neuronal expression of SPOCK1 it is possible, that SPOCK1 is involved in the regulation of cardiac innervation.

Thus, according to this invention SPOCK1 could potentially contribute to an increased risk of CHD such as AMI and other cardiovascular diseases due to modulation of immune response as well as lysosomal degradation of necrotic tissue following ischemia, e.g. myocardial or cerebral ischemia.

As part of this invention, we have identified a SNP marker rs6826647, located 3,174 bp from the 3 prime end of the SPOCK3 gene, which is significantly associated with several cardiovascular diseases and metabolic syndrome related traits. In the studied population, subjects homozygous for the minor allele T (allele frequency about 0.369) of the SNP rs6826647 had significantly increased blood pressure (p=0.000), elevated levels of blood glucose (p=0.029) and plasma insulin (p=0.028), which follow the classical pre-diabetic state. Furthermore, the SNP rs6826647 was associated with tendency for increased BMI (p=0.052).

Thus, the SPOCK3 may modulate activity of enzymes involved in the regulation of the studied traits, such as blood pressure. Alternatively, any SPOCK3 gene product could directly or indirectly regulate insulin secretion in β-cells of Langerhans islands by regulating innervation related to regulation of β-cell activity. Furthermore, any SPOCK3 protein isoform may be involved in mediating the transmission of blood glucose level singal to insulin receptors, and thereby playing a role in type 2 diabetogenic mechanisms. SPOCK3 may have also other functions than those mentioned above in the studied traits.

Methods of Therapy

The present invention discloses novel methods for the prevention and treatment of CHD, AMI, HT, MBO and obesity based on modification of expression or activity or function of proteins and polypeptides regulating peptidases, and endogenous and exogenous modulators of said genes, proteins and polypeptides. The term, “treatment” as used herein, refers not only to ameliorating symptoms associated with the disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition.

The present invention encompasses methods of treatment (prophylactic and/or therapeutic) of CHD, AMI, HT, MBO and obesity, for such individuals as in the target populations described herein, using a therapeutic agent. “Therapeutic agents” of this invention comprises agents that alter (e.g., enhance or inhibit) enzymatic activity or fumction of one or more biological networks and/or metabolic pathways related to SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or/and SPOCK3 polypeptides and proteins. These agents may also also enhance or reduce the activity and/or expression of SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or/and SPOCK3 genes as well as their encoded proteins and polypeptides.

Representative therapeutic agents comprise the following: (a) nucleic acids, fragments, variants or derivatives of SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 genes; (b) nucleic acids encoding SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 polypeptides, their active fragments, variants or derivatives thereof; (c) and nucleic acids modifying the expression of SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 genes (e.g. antisense polynucleotides, catalytically active polynucleotides (e.g. ribozymes and DNAzymes), molecules inducing RNA interference (RNAi) and micro RNA); (d)vectors comprising said nucleic acids; (e) SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 polypeptides, active fragments, variants or derivatives thereof; (f) SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 polypeptide binding agents; peptidomimetics; fusion proteins and prodrugs thereof; (g) monoclonal and polyclonal antibodies to mutant or non-mutant SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 polypeptides; (h) polypeptide substrates of SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 polypeptides; (i) metabolites of SERPINB 1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 and SPOCK3 polypeptides or derivatives thereof; (j) small molecules and compounds that inhibit or antagonize SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or/and SPOCK3 polypeptides or related biochemical networks and/or metabolic pathways and (k) small molecules and compounds that induce or agonize SERPIB1, SERPINB2, SERPINB3, SERPINB4, SERPINB5, SERPINB7, SERPINB8, SERPINB9, SERPINB 11, SERPINB12, SERPINB13, SPINK5, SPINK5L2, SPINK5L3, SPOCK, SPOCK2, TLL1 or/and SPOCK3 polypeptides or related biochemical networks and/or metabolic pathways.

The useful therapeutic agents of this invention are designed to compensate the observed alterations in activity and/or function of SERPIB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or/and SPOCK3 polypeptides or related biological networks and metabolic pathways. The therapeutic agents may act on SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or/and SPOCK3 genes or their encoded polypeptides by a variety of means: for example, by altering translation rate, by altering transcription rate, by altering posttranslational processing rate, by interfering with polypeptide activity and/or function (e.g., by binding to a polypeptide), by altering stability of polypeptides, by altering the transcription rate of splice variants, by inhibiting or enhancing transfer of polypeptides in target cells, organs and/or tissues or by inhibiting or enhancing related biological networks and metabolic pathways.

More than one therapeutic agent can be used concurrently, if desired. The therapy is designed to alter (e.g., inhibit or enhance), replace or supplement activity and/or function of one or more of the SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or/and SPOCK3 genes or their encoded polypeptides or related metabolic pathways in an individual. For example, a therapeutic agent can be administered in order to increase or decreasethe expression or availability of a gene encoded polypeptide or a specific variant. For example by increasing expression or availability of a biologically active native or variant polypeptide it is possible to interfere with or compensate for the expression or activity of a defective gene or variant.

The therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 gene), either by itself or included within a vector, can be introduced into cells of an individual affected by CHD, AMI, HT, MBO or/and obesity using variety of experimental methods described in the art, so that the treated cells start to produce SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 polypeptide. Thus, cells which, in nature, lack expression and activity or have abnormal expression and activity of SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3, can be engineered to express a SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 polypeptide or an active fragment or a different variant thereof. Genetic engineering of cells may be done either “ex vivo” (i.e. suitable cells are isolated and purified from a patient and re-infused back to the patient after genetic engineering) or “in vivo” (i.e. genetic engineering is done directly to a tissue of a patient using a vehicle).

Alternatively, in another embodiment of the invention, a nucleic acid of the invention; a nucleic acid complementary to a nucleic acid of the invention; or a portion of such a nucleic acid (e.g., a polynucleotide), can be used in “antisense” therapy, in which a nucleic acid (e.g., a polynucleotide) which specifically hybridizes to the mRNA and/or genomic DNA of SERPINB1, SERPINB2, SERPINB3, SERPINB4, SERPINB5, SERPINB7, SERPINB8, SERPINB9, SERPINB11, SERPINB12, SERPINB13, SPINK5, SPINKSL2, SPINK5L3, SPOCK, SPOCK2, TLL1 or SPOCK3 gene is administered in a pharmaceutical composition to the target cells or said nucleic acid is generated “in vivo”. The antisense nucleic acid that specifically hybridizes to the mRNA and/or DNA inhibits expression of the SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 polypeptide, e.g., by inhibiting translation and/or transcription. Binding of the antisense nucleic acid can be due to conventional base pairing, or, for example, in the case of binding to DNA duplexes, through specific interaction in the major groove of the double helix.

In a preferred embodiment nucleic acid therapeutic agents of the invention are delivered into cells that express one or more of the SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 genes. A number of methods including, but not limited to, the methods known in the art can be used for delivering a nucleic acid to said cells. For example, a vector can be introduced in vivo such that it is taken up by a cell and directs the transcription of a RNA molecule, which induces RNA interference in the cell. Such a vector can remain episomal or become chromosomally integrated, and as long as it can be transcribed to produce the desired RNA molecules it will modify the expression of the endogenous SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 gene. Such vectors can be constructed by various recombinant DNA technology methods standard in the art.

The expression of an endogenous SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 gene can be also reduced by inactivating or “knocking out” using targeted homologous recombination methods described in the art. Alternatively, expression of a functional, non-mutant SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 gene can be increased using a similar method: targeted homologous recombination can be used to replace a non-functional gene with a functional form of the said gene in a cell.

In yet another embodiment of the invention, other therapeutic agents as described herein can also be used in the treatment or prevention of CHD, AMI, HT, MBO or/and obesity. The therapeutic agents can be delivered in a pharmaceutical composition to be administered systemically, or can be targeted to a particular tissue. The therapeutic agents can be produced by a variety of means, including chemical synthesis, cell culture and recombinant techniques (e.g. with transgenic cells and animals). Therapeutic agents can be isolated and purified to fulfill pharmaceutical requirements using standard methods described in the art.

A combination of any of the above methods of treatment (e.g., administration of non-mutant SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 polypeptide in conjunction with RNA molecules inducing RNA interference targeted to the mutant SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 mRNA) can also be used.

In another embodiment of the invention, pharmaceutical therapy of the invention comprises compounds, which enhance or reduce the activity and/or fuinction of one or several biological networks and/or metabolic pathways related to SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 genes, proteins or polypeptides. The treatment may also enhance or reduce the expression of one or several genes in said biological networks and/or metabolic pathways.

The invention also discloses methods and test kits for risk assessment, diagnosis or prognosis of CHD, AMI, HT, MBO and obesity in an individual. Such methods and test kits are useful when selecting prophylactic treatment and/or drug therapy for individuals having a high risk of CHD, AMI, HT, MBO and obesity, or who might have increased risk for adverse effects of drugs affecting SERPINB1, SERPINB2, SERPINB3, SERPPB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 genes or their encoded polypeptides.

Pharmaceutical Compositions

The present invention also pertains to pharmaceutical compositions comprising agents described herein, particularly polynucleotides, polypeptides and any fractions, variants or derivatives of SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 genes, and/or agents that alter (e.g., enhance or inhibit) expression of SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 genes, or activity of one or more polypeptides encoded by SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 genes as described here. The agents of the present invention can be formulated with a physiologically acceptable carrier or excipient to prepare a pharmaceutical composition. The carrier and composition can be sterile. The formulation should suit the mode of administration.

In a preferred embodiment pharmaceutical compositions comprise agent or agents reversing, at least partially, CHD, AMI, HT, MBO and/or obesity associated changes in biological networks and/or metabolic pathways related to the SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 genes of this invention.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of the disease, and should be decided according to the judgment of a practitioner and each patient's circumstances. Effective doses may be extrapolated from dose-response curves derived from in vitro or animal model test systems.

Representative Target Population

An individual at risk of CHD, AMI, HT, MBO and/or obesity is an individual who has at least one risk factor, such as family history of CHD, AMI, HT, MBO and/or obesity, previously identified glucose intolerance, obesity, hypertriglyceridemia, low HDL cholesterol, HT and elevated BP. The detection method of the invention may also further comprise a step determining blood, serum or plasma glucose, total cholesterol, HDL cholesterol, LDL cholesterol, triglyceride, apolipoprotein B and AI, fibrinogen, ferritin, transferrin receptor, C-reactive protein, serum or plasma insulin concentration or a disease risk allele or haplotype.

In another embodiment of the invention, an individual who is at risk of CHD, AMI, HT, MBO and/or obesity is an individual who is having at least one CHD, AMI, HT, MBO and/or obesity risk associated biomarker in SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes, in which the presence of the biomarker is indicative of a susceptibility to CHD, AMI, HT, MBO and/or obesity. The term “gene,” as used herein, refers to an entirety containing all regulatory elements located both upstream and downstream as well as within of a polypeptide encoding sequence of a gene and entire transcribed region of a gene including 5′ and 3′ untranslated regions of mRNA and the entire polypeptide encoding sequence including all exon and intron sequences (also alternatively spliced exons and introns) of a gene.

Assessment for Disease Risk Alleles and Haplotypes

The genetic markers are particular “alleles” at “polymorphic sites” associated with CHD, AMI, HT, MBO and/or obesity. A nucleotide position in genome at which more than one sequence is possible in a population, is referred to herein as a “polymorphic site”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a reference nucleotide sequence is referred to for a particular gene e.g. in NCBI databases (www.ncbi.nlm.nih.gov). Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences.

Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal MRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a disease susceptibility gene. For example, a nucleotide change resulting in a change in polypeptide sequence can alter the physiological properties of a polypeptide dramatically by resulting in altered activity, distribution and stability or otherwise affect on properties of a polypeptide.

Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the MRNA of a gene may result in altered translation of the MRNA e.g. by inducing stable secondary structures to the MRNA and affecting the stability of the mRNA. Such sequence changes may alter the expression of a disease susceptibility gene.

A “haplotype,” as described herein, refers to any combination of genetic markers (“alleles”). A haplotype can comprise two or more alleles and the length of a genome region comprising a haplotype may vary from few hundred bases up to hundreds of kilobases. As it is recognized by those skilled in the art the same haplotype can be described differently by determining the haplotype defining alleles from different nucleic acid strands. E.g. the haplotype CGAA defined by the SNP markers rs6992115, rs868586, rs13254457, and rs1467108 is the same as the haplotype GCTT in which the SNP markers rs6992115, rs868586, rs13254457, and rs1467108 are determined from the complementary strand or haplotype GGAA in which the SNP marker rs6992115 is determined from the complementary strand. The haplotypes described herein are found more frequently either from diseased individials (risk increasing haplotypes) or from disease free individuals (protective haplotypes). Therefore, these haplotypes have predictive value for CHD, AMI, HT, MBO and/or obesity in an individual. Detecting haplotypes can be accomplished by methods known in the art for detecting sequences at polymorphic sites.

It is understood that the disease associated risk increasing and protective haplotypes described in this invention may be associated with other “polymorphic sites”. These other polymorphic sites may reside in same genes as the described haplotypes or in other genes and these other polymorphic sites may be either equally usefull as genetic markers or even more useful as causative variations explaining the observed disease association of alleles and haplotypes of this invention.

In certain methods described herein, an individual who is at risk for a disease is an individual in whom a disease risk allele or disease risk haplotype is identified. In one embodiment, the disease risk allele or disease risk haplotype is one that confers a significant risk of death. In one embodiment, significance associated with an allele or a haplotype is measured by an odds ratio. In a further embodiment, the significance is measured by a percentage. In one embodiment, a significant risk is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the allele or the haplotype, and often, environmental factors.

Primers, Probes and Nucleic Acid Molecules

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Such probes and primers include polypeptide nucleic acids (Nielsen PE et al, 1991).

A probe or primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence.

In preferred embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

Antisense nucleic acid molecules of the invention can be designed using the nucleotide sequences of SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes and/or their complementary sequences and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule encoding SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 or SPOCK3 gene, a fragment or a variant thereof has been cloned in antisense orientation (i.e., RNA transcribed from the expression vector will be complementary to the transcribed SERPINB1, SERPINB2, SERPINB3, SERPINB4, SERPINB5, SERPINB7, SERPINB8, SERPINB9, SERPINB11, SERPINB12, SERPINB13, SPINK5, SPINK5L2, SPINK5L3, SPOCK, SPOCK2, TLL1 or SPOCK3 RNA of interest).

The nucleic acid sequences of the SERPNB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes described in this invention can also be used to compare with endogenous DNA sequences in patients to make risk assessment, diagnosis or prognosis of CHD, AMI, HT, MBO and obesity, and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Polyclonal and Monoclonal Antibodies

The invention comprises polyclonal and monoclonal antibodies that bind to polypeptides of the invention. The term “antibody” as used herein refers to immunoglobulin molecules and immunologically active portions of immunoglobulin molecules, i.e., molecules that contain a binding site that specifically binds to an epitope (antigen, antigenic determinant). An antibody molecule that specifically binds to a polypeptide of the invention is a molecule that binds to an epitope present in said polypeptide or a fragment thereof, but does not substantially bind other molecules in a sample, e.g., a biological sample, which naturally contains the polypeptide. Examples of immunologically active portions of immunoglobulin molecules include F(ab) and F(ab′).sub.2 fragments which can be generated by treating the antibody with an enzyme such as pepsin. Polyclonal and/or monoclonal antibodies that specifically bind one form of the gene product but not to the other form of the gene product are also provided. Antibodies are also provided, that bind a portion of either the variant or the reference gene product that contains the polymorphic site or sites. The term “monoclonal antibody” or “monoclonal antibody composition”, as used herein refers to a population of antibody molecules that are directed against a specific epitope and are produced either by a single clone of B cells or a single hybridoma cell line. A monoclonal antibody composition thus typically displays a single binding affinity for a particular polypeptide of the invention with which it immunoreacts.

Polyclonal antibodies can be prepared as known by those skilled in the art by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique (Kohler G and Milstein C, 1975), the human B cell hybridoma technique (Kozbor D et al, 1982), the EBV-hybridoma technique (Cole SP et al, 1984), or trioma techniques (Hering S et al, 1988). To produce a hybridoma an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (Bierer B et al, 2002). Moreover, the ordinarily skilled worker will appreciate that there are many variations of such methods that also would be useful. Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide (Hayashi N et al, 1995; Hay B N et al, 1992; Huse W D et al, 1989; Griffiths A D et al, 1993). Kits for generating and screening phage display libraries are commercially available.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. An antibody specific for a polypeptide of the invention can facilitate the purification of a native polypeptide of the invention from biological materials, as well as the purification of recombinant form of a polypeptide of the invention from cultured cells (culture media or cells). Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein and/or metabolite levels in tissues such as blood as part of a risk assessment, diagnostic or prognostic test for CHD, AMI, HT, MBO and obesity or as part of a clinical testing procedure, e.g., to, for example, determine the efficacy of a given treatment regimen. Antibodies can be coupled to various enzymes, prosthetic groups, fluorescent materials, luminescent materials, bioluminescent materials, and radioactive materials to enhance detection. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include .sup.125I, 131I, 35S or 3H.

Highly purified antibodies (e.g. monoclonal humanized antibodies specific to a polypeptide encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes of this invention) may be produced using GMP-compliant manufacturing processes well known in the art. These “pharmaceutical grade” antibodies can be used in novel therapies modulating activity and/or function of a polypeptide encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 or SPOCK3 gene of this invention or modulating activity and/or function of a metabolic pathway related to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes of this invention.

Diagnostic Assays

The markers, probes, primers and antibodies described herein can be used in methods and kits used for risk assessment, diagnosis or prognosis of CHD, AMI, HT, MBO and obesity in a subject. The methods and test kits of this invention comprise biomarkers associated to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes, their encoded polypeptides and related biochemical networks and metabolic pathways.

The invention also discloses methods for selecting and monitoring treatment e.g. drug therapy and selecting subjects testing treatments for CHD, AMI, HT, stroke, MBO and obesity by assessing biomarkers associated to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes, their encoded polypeptides and related biochemical networks and metabolic pathways.

A test assessing biomarkers associated to expression of SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes is useful in selecting drug therapy for patients who might be at increased risk for adverse effects of drugs affecting to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 activities. The same tests are useful also in selecting supplementation affecting serine or cysteine peptidases or their substrates; either by avoiding the use of drugs affecting SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 or SPOCK3 activities or by including specific drugs affecting SERPINB1,SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 or SPOCK3 activities in their therapeutic regimen.

In one embodiment of the invention, risk assessment, diagnosis or prognosis of cardiovascular diseases such as CHD, AMI, HT, and metabolic disorders such as MBO and obesity is made by detecting one or several of at-risk alleles or at-risk haplotypes or a combination of at-risk alleles and at-risk haplotypes described in this invention in the subject's nucleic acid as described herein.

In another embodiment of the invention, risk assessment, diagnosis or prognosis of cardiovascular diseases such as CHD, AMI, HT, and metabolic disorders such as MBO and obesity is made by detecting one or several of polymorphic sites which are associated with at-risk alleles or/and at-risk haplotypes described in this invention in the subject's nucleic acid. Diagnostically the most useful polymorphic sites are those altering the polypeptide structure of a gene due to a frame shift; due to a premature stop codon, due to an amino acid change or due to abnormal MRNA splicing. Nucleotide changes resulting in a change in polypeptide sequence in many cases alter the physiological properties of a polypeptide by resulting in altered activity, distribution and stability or otherwise affect on properties of a polypeptide. Other diagnostically useful polymorphic sites are those affecting transcription of a gene or translation of it's MRNA due to altered tissue specificity, due to altered transcription rate, due to altered response to physiological status, due to altered translation efficiency of the MRNA and due to altered stability of the MRNA.

For diagnostic applications, there may be polymorphisms informative for prediction of disease risk, which are in linkage disequilibrium with the functional polymorphism. Such a functional polymorphism may alter splicing sites, affect the stability or transport of MRNA, or otherwise affect the transcription or translation of the nucleic acid. The presence of nucleotide sequence variants associated with a functional polymorphism is diagnostic for susceptibility to cardiovascular diseases such as CHD, AMI, HT, and metabolic disorders such as MBO and obesity. While we have genotyped and included a limited number of example SNP markers in the experimental section, any fuictional, regulatory or other mutation or alteration described above in any of the SERPINB1, SERPINB2, SERPMB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes identified herein is expected to predict the risk of CHD, AMI, HT, MBO and obesity.

In diagnostic assays determination of the nucleotides present in one or several of the SNP markers of this invention, as well as polymorphic sites associated with the SNP markers of this invention, in an individual's nucleic acid can be done by any method or technique which can accurately determine nucleotides present in a polymorphic site. Numerous suitable methods have been described in the art (see e.g. Kwok P-Y, 2001; Syvänen A-C, 2001), these methods include, but are not limited to, hybridization assays, ligation assays, primer extension assays, enzymatic cleavage assays, chemical cleavage assays and any combinations of these assays. The assays may or may not include PCR, solid phase step, a microarray, modified oligonucleotides, labeled probes or labeled nucleotides and the assay may be multiplex or singleplex. As it is obvious in the art the nucleotides present in a polymorphic site can be determined from either nucleic acid strand or from both strands.

In another embodiment of the invention, risk assessment, diagnosis or prognosis of CHD, AMI, HT, MBO and obesity can be assessed by examining transcription of SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes. Alterations in transcription can be assessed by a variety of methods described in the art, including e.g. hybridization methods, enzymatic cleavage assays, RT-PCR assays and microarrays. A test sample from an individual is collected and the alterations in the transcription of SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes are assessed from the RNA present in the sample. An altered transcription profile when compared to healthy control subjects is diagnostic for CHD, AMI, HT, MBO and obesity.

In another embodiment of the invention, risk assessment, diagnosis or prognosis of CHD, AMI, HT, MBO and obesity can also be made by examining expression and/or structure and/or function of SERPINB1, SERPINB2, SERPINB3, SERPINB4, SERPINB5, SERPINB7, SEPRINB8, SERPINB9, SERPINB11,SERPINB12, SERPINB13, SPINK5, SPINK5L2, SPINK5L3, SPOCK, SPOCK2, TLL1 and SPOCK3 polypeptides. A test sample from an individual is assessed for the presence of alterations in the expression and/or structure and/or function of polypeptides encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes. An alteration in expression of a polypeptide can be, for example, quantitative (an alteration in the quantity of the expressed polypeptide, i.e., the amount of polypeptide produced) or qualitative (an alteration in the structure and/or function of a polypeptide encoded, i.e. expression of a mutant polypeptide or of a different splicing variant or isoform).

Alterations in expression and/or structure and/or function of SERPNB 1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 polypeptides can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition in a control sample and an alteration can be assessed either directly from the SERPINB1, SERPINB2, SERPINB3, SERPINB4, SERPINB5, SERPINB7, SEPRINB8, SERPINB9, SERPINB11,SERPINB12, SERPINB13, SPINK5, SPINK5L2, SPINK5L3, SPOCK, SPOCK2, TLL1 and SPOCK3 polypeptides or their fragments or from their substrates and reaction products. A control sample is a sample that corresponds to the test sample (e.g., is from the same type of cells), and is from an individual who is not affected by the diseases of interest. An alteration in the expression or composition of a polypeptide encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes of the invention in the test sample, as compared with the control sample, is indicative CHD, AMI, HT, MBO and obesity.

Immunological analyses such as Western blotting analysis, using an antibody as described above that specifically binds to a polypeptide encoded by a mutant SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 or SPOCK3 gene or an antibody that specifically binds to a polypeptide encoded by a non-mutant gene, or an antibody that specifically binds to a particular splicing variant encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 or SPOCK3 gene can be used to identify the presence or absence of a particular polypeptide encoded by a polymorphic or mutant gene in a test sample. The presence of a polypeptide encoded by a polymorphic or mutant gene, or the absence of a polypeptide encoded by a non-polymorphic or non-mutant gene, is diagnostic for CHD, AMI, HT, MBO and obesity, as is the presence (or absence) of particular splicing variants.

In one embodiment of this invention, the level or amount of polypeptides encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes in a test sample are compared with the level or amount of the same polypeptides in a control sample. A level or amount of the polypeptides in the test sample that are higher or lower than the level or amount of the same polypeptides in the control sample, such that the difference is statistically significant, are indicative of altered expression of SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes. The biomarkers associated with altered expression are applicable in risk assessment, diagnosis and prognosis of CHD, AMI, HT, MBO and obesity. Alternatively, the composition of the polypeptides encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes in a test sample are compared with a control sample (e.g., the presence of different splicing variants and mutants). A difference in the composition of the polypeptides of the test sample, as compared with the composition of the polypeptides of the control sample is applicable in risk assessment, diagnoisis and prognosis of CHD, AMI, HT, MBO and obesity. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is diagnostically applicable.

In another embodiment, assessment of the splicing variant or isoform(s) of a polypeptide encoded by a polymorphic or mutant SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 or SPOCK3 gene can be performed. The assessment can be performed directly (e.g., by examining the polypeptide itself), or indirectly (e.g., by examining the MRNA encoding the polypeptide, such as through mRNA profiling). For example, probes and primers as described herein can be used to determine which splicing variants or isoforms are encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 gene mRNAs, using standard methods.

The presence in a test sample of a particular splicing variant(s) or isoform(s) of SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes associated with CHD, AMI, HT, MBO and obesity is diagnostic. Similarly, the absence in a test sample of a particular splicing variant(s) or isoform(s) associated with CHD, AMI, HT, MBO and obesity, or the presence in a test sample of a particular splicing variant(s) or isoform(s) not associated with CHD, AMI, HT, MBO and obesity, is diagnostic for the absence of disease or condition.

The invention further pertains to a method for the diagnosis and identification of susceptibility to CHD, AMI, HT, MBO and obesity in an individual, by assessing markers present in at-risk alleles or at-risk haplotypes of SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes. In one embodiment, the at-risk allele or the at-risk haplotype is an allele or a haplotype for which the presence of the allele or the haplotype increases the risk of CHD, AMI, HT, MBO or obesity significantly. Although it is to be understood that identifying whether a risk is significant may depend on a variety of factors, including the specific disease, the haplotype, and often, environmental factors, the significance may be measured by an odds ratio or a percentage. In a further embodiment, the significance is measured by a percentage. In one embodiment, a significant risk is measured as an odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, an odds ratio of at least 1.2 is significant. In a further embodiment, an odds ratio of at least about 1.5 is significant. In a further embodiment, a significant increase or decrease in risk is at least about 1.7. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase or reduction in risk is at least about 50%. It is understood however, that identifying whether a risk is medically significant may also depend on a variety of factors, including the specific disease, the allele or the haplotype, and often, environmental factors.

The invention also pertains to methods of risk assessment, diagnosis or prognosis to CHD, AMI, HT, MBO and obesity in an individual, comprising screening for an at-risk haplotypes in SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes that are more frequently present in an individual susceptible to CHD, AMI, HT, MBO and obesity (affected), compared to the frequency of its presence in a healthy individual (control), wherein the presence of the haplotypes are indicative CHD, AMI, HT, MBO and obesity.

Yet in another embodiment risk assessment, diagnosis or prognosis of CHD, AMI, HT, MBO and obesity in an individual is performed by assessing the status and/or activity of SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 related biological networks and/or metabolic pathways. Status and/or function of a biological network and/or a metabolic pathway can be assessed e.g. by measuring amount or composition of one or several polypeptides or metabolites belonging to the biological network and/or to the metabolic pathway from a biological sample taken from a subject. Risk assessment, diagnosis or prognosis of CHD, AMI, HT, MBO and obesity is done by comparing observed status and/or activities of biological networks and/or metabolic pathways of a subject to the status and/or activities of the same biological networks and/or metabolic pathways of healthy controls.

Kits (e.g., reagent kits) useful in the various described methods of risk assessment, diagnosis and/or prognosis of CHD, AMI, HT, MBO and obesity assess biomarkers related to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes. Useful components of such kits include for example SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 gene related PCR primers, allele-specific oligonucleotides, hybridization probes or primers as described herein (e.g., labeled probes or primers) and antibodies which bind to altered or to non-altered (native) polypeptides. Other examples of useful components of such kits are reagents for genotyping SNP markers, reagents for detection of labeled molecules, restriction enzymes (e.g., for RFLP analysis), DNA polymerases, RNA polymerases, marker enzymes, means for amplification of nucleic acids comprising one or more genes of this invention, or means for analyzing the nucleic acid sequence of one or more genes of this invention or for analyzing the amino acid sequence of one or more polypeptides of this invention. In one embodiment, a kit for risk assessment, diagnosis and/or prognosis of CHD, AMI, HT, MBO or obesity can comprise primers for nucleic acid amplification of fragments from SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes comprising markers defining an at-risk haplotype that is more frequently present in an individual susceptible to CHD, AMI, HT, MBO or obesity. The primers can be designed using portions of the nucleic acid sequence flanking SNPs that are indicative of CHD, AMI, HT, MBO or obesity.

The methods and kits of the invention may further comprise a step of combining personal and clinical information concerning e.g. age, gender, smoking status, physical activity, waist-to-hip circumference ratio (cm/cm), the subject family history of CHD, AMI, HT, MBO and obesity, previously identified glucose intolerance, obesity, hypertriglyceridemia, low HDL cholesterol, HT and elevated BP. The detection method of the invention may also further comprise a step determining blood, serum or plasma glucose, total cholesterol, HDL cholesterol, LDL cholesterol, triglyceride, apolipoprotein B and Al, fibrinogen, ferritin, transferrin receptor, C-reactive protein, serum or plasma insulin concentration.

The score that predicts the probability of having CHD, AMI, HT, MBO and/or obesity may be calculated e.g. using a multivariate failure time model or a logistic regression equation. The results from the further steps of the method as described above render possible a step of calculating the probability of CHD, AMI, HT, MBO and/or obesity using a logistic regression equation as follows.

Probability of CHD, AMI, HT, MBO and/or obesity=1/[1+e (−(−a+Σ(bi*Xi))], where e is Napier's constant, Xi are variables related to the CHD, AMI, HT, MBO and/or obesity, bi are coefficients of these variables in the logistic finction, and a is the constant term in the logistic function, and wherein a and bi are preferably determined in the population in which the method is to be used, and Xi are preferably selected among the variables that have been measured in the population in which the method is to be used. Preferable values for bi are between −20 and 20; and for i between 0 (none) and 100,000. A negative coefficient bᵢ implies that the marker is risk-reducing and a positive that the marker is risk-increasing. Xi are binary variables that can have values or are coded as 0 (zero) or 1 (one) such as SNP markers. The model may additionally include any interaction (product) or terms of any variables Xi, e.g. biXi. An algorithm is developed for combining the information to yield a simple prediction of CHD, AMI, HT, MBO and/or obesity as percentage of risk in one year, two years, five years, 10 years or 20 years. Alternative statistical models are failure-time models such as the Cox's proportional hazards' model, other iterative models and neural networking models.

Monitoring Progress of Treatment

The current invention also pertains to methods of monitoring the effectiveness of a treatment of CHD, AMI, HT, MBO and/or obesity by assessing expression (e.g., relative or absolute expression) of SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes. The expression of genes can be assessed from their mRNAs or from the amount and activity of their expressed polypeptides in a tissue sample (e.g. peripheral blood sample or adipose tissue biopsy). An assessment of the levels of expression or biological activity of the polypeptide can be made before and during treatment with CHD, AMI, HT, MBO and/or obesity therapeutic agents.

Alternatively the effectiveness of a treatment of CHD, AMI, HT, MBO and/or obesity can be followed by assessing the status and/or function of biological networks and/or metabolic pathways related to one or more polypeptides encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes of this invention. Status and/or function of a biological network and/or a metabolic pathway can be assessed e.g. by measuring amount or composition of one or several polypeptides, belonging to the biological network and/or to the metabolic pathway, from a biological sample taken from a subject before and during a treatment. Alternatively status and/or function of a biological network and/or a metabolic pathway can be assessed by measuring one or several metabolites belonging to the biological network and/or to the metabolic pathway, from a biological sample before and during a treatment. Effectiveness of a treatment is evaluated by comparing observed changes in status and/or function of biological networks and or metabolic pathways following treatment with CHD, AMI, HT, MBO and/or obesity therapeutic agents to the data available from healthy subjects.

In addition, DNA sequence variations such as SNP markers defining haplotypes or mutations within or near (e.g. within 1 to 200 kb) SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes may be used to identify individuals who are at higher risk for CHD, AMI, HT, MBO and/or obesity to increase the power and efficiency of clinical trials for pharmaceutical agents to prevent or treat CHD, AMI, HT, MBO and/or obesity. The presence of at-risk haplotypes and other variations may be used to exclude or fractionate patients in a clinical trial who are likely to have involvement of another pathway in their CHD, AMI, HT, MBO and/or obesity in order to enrich patients who have pathways involved that are relevant regarding to the treatment tested and boost the power and sensitivity of the clinical trial. Such variations may be used as a pharmacogenetic test to guide the selection of pharmaceutical agents for individuals.

### EXAMPLE 1.

KIHD Cohort Genotyping Study

Study Design

This invention is based on “familial case-control” whole-genome association study approach, in which patterns of genetic markers in patients (the “cases”) and controls are defined, and differences in markers and haplotypes between the cases and controls are analyzed. These indicate disease associated loci. To be able to study multiple diseases simultaneously, the controls were selected so that they had neither personal medical history nor family history of either CHD or HT. The cases were selected initially of persons with family history of CHD who had experienced AMI during a long follow-up period from the 1980's through 2000's. Thus, the study design for AMI is a prospective nested case-control study and for HT and quantitative traits, a cross-sectional study. This work is based on 250 subjects, 246 of whom were men.

Genetic Homogeneity of the East Finland Founder Population

Both Y-chromosomal haplotypes and mitochondrial sequences show low genetic diversity among Finns compared with other European populations and confirm the long-standing isolation of Finland. During the 16^{th} century when Finland was still part of Sweden, internal migrations from the settled coastal areas created regional sub-isolates within the population. During King Gustavus of Vasa (1523-1560) over 400 years ago, internal migrations created regional subisolates, the late settlements (Varilo et al 2000).The East Finland isolate, which was founded by approximately 20-30 families and has grown rather rapidly over 15 or so generations, remained rather isolated as a consequence of distance, language, religion and culture influences. Therefore, even today the population exhibits a rare degree of genetic homogeneity.

The East Finnish population is the most genetically-homogenous population isolate known that is large enough for effective gene discovery program. The reasons for homogeneity are:
1. the young age of the population (fewer generations),
2. the small number of founders,
3. long-term geographical isolation, and
4. population bottlenecks because of wars, famine and fatal disease epidemics.
   Study Population and Phenotype Characterization
   Study Population The subjects were participants of the Kuopio Ischaemic Heart Disease Risk Factor Study (KIHD), which is an ongoing prospective population-based study designed to investigate risk factors for chronic diseases, including AMI, CHD, HT, stroke, T2D, MBO and obesity, among middle-aged men from East Finland, initiated by Jukka T. Salonen, the current principal inventor (Salonen J T 1988, Salonen J T et al 1998, Tuomainen T-P et al 1999). The study population was a random age-stratified sample of men living in Eastern Finland who were 42, 48, 54 or 60 years old at baseline examinations in 1984-1989. A total of 2682 men were examined in the baseline examinations during 1984-89. Data used here concerning HT, T2D, obesity, MBO and the quantitative traits were obtained from this baseline examination. The male cohort was complemented by a random population sample of 920 women, first examined during 1998-2001, at the time of the 11 -year follow up of the male cohort. The follow-up of AMIs and strokes was to the end of 2002 (2003 for CHD deaths), providing a theoretical follow-up time ranging from 13 (14) years to 18 (19) years. The average follow-up time was 14.3 years (range 0.4 to 17.7 years for individual subjects). The recruitment and examination of the subjects has been described previously in detail (Salonen J T 1988, WO 02/074230, W003/089638). The Kuopio University and University Hospital Research Ethics Committee approved the KIHD study. All participants gave their written informed consent.
   AMI

Data on CHD and AMI during the follow-up were obtained by computer record linkage to the national computerized hospital discharge registry. Diagnostic information was collected from the hospitals and all heart attacks events were classified according to rigid predefined criteria. The diagnostic classification of acute coronary events was based on symptoms, electrocardiographic findings, cardiac enzyme elevations, autopsy findings and the history of CHD. Each suspected coronary event (ICD-9 codes 410-414 and ICD-10 codes I20-I25) was classified into 1) a definite AMI, 2) a probable AMI, 3) a typical acute chest pain episode of more than 20 minutes indicating CHD, 4) an ischemic cardiac arrest with successful resuscitation, 5) no acute coronary event or 6) an unclassifiable fatal case. The categories 1) to 3) were combined for the present analysis to denote AMI. If a subject had multiple non-fatal events during the follow-up, the first AMI was considered as the end point.

The cases were defined so that they had either a confirmed definite or probable AMI or typical prolonged chest pain and a family history of AMI (at least one affected family member, either a sibling or a parent). These characteristics were determined to increase the likelihood that the coronary disease in the case subjects was caused by genes and not by non-genetic factors. Analogically, the controls did not have family history of AMI in either their parents of siblings.

An identical number of healthy control subjects were selected from the same KIHD cohort as the cases. They had no family history of CHD in parents or siblings. To minimize the control-dilution bias (controls developing AMI later), CHD-free controls were selected from very healthy persons. The controls were free of CHD, assessed broadly. The controls for GWS had neither diagnosed CHD, symptoms or signs of CHD, nitroglycerin medication, ischaemic ECG findings in maximal exercise test, type 2 diabetes nor moderate-to-severe hypertension. The proportion of males was equal among both the cases and the controls. To control for confounding, the controls were matched according to gender, smoking status and the municipality of residence. In this founder-population-based familial case-control design, the number of both the cases and the controls used in the initial GWS was 125 (125+125 =250). As the controls had been matched, the age and the number of cigarettes smoked daily were similar in both groups.

CHD Death

Forty of the 125 patients who experienced AMI during the follow-up also died of CHD during the follow-up up to the end of 2003. Coronary death was defined as death for which the underlying cause was determined to be ICD-9 code 410-414 or ICD-10 code I20-I25. In the statistical analysis, the 40 case subjects who died of CHD were compared with all other 206 male subjects who did not die of CHD during the follow-up. Eighty-five of these had experienced a non-fatal AMI during the follow-up and 121 men remained free of CHD during the follow-up. The main statistical analyses were repeated after exclusion of the 85 men who experienced a non-fatal AMI. As the findings were virtually identical, the results of those analyses will not be presented.

Prevalent CHD

Prevalent CHD was defined as either self-reported history of AMI or other CHD, the presence of either angina pectoris on effort according to the London School of Hygiene questionnaire, regular use of sublingual nitroglycerin tablets or ischemia during exercise test. Exercise ischemia was defined as either typical chest pain or ischemic ECG changes during or after the exercise test. A maximal symptom-limited exercise tolerance test was performed using an electrically-braked cycle ergometer. The electrocardiogram (ECG) was registered continuously during the test. The ECG criteria for ischemia during exercise were horizontal or downsloping ST depression ≧1.0 mm at 80 msec after J point or any ST depression of more than 1.0 mm at 80 msec after J point.

Hypertension

Resting blood pressure was measured by an experienced nurse using a random-zero sphygmomanometer (cuff size 14×54 cm, Hawksley, Lancing, United Kingdom) after 5 and 10 minutes of rest in a seated position in a quiet room between 8:00 a.m. and 10:00 a.m. The measuring protocol included three measurements in supine, one in standing and two in sitting position with 5-minutes intervals. The mean of all six measurements were used as SBP and DBP. Moderate-to-severe hypertension was defined as either systolic blood pressure (SBP)≧165 mmHg or diastolic BP (DBP) ≧95 mmHg or antihypertensive treatment. Mild-to-severe hypertension was defined as either systolic blood pressure (SBP) ≧140 mmHg or diastolic BP (DBP) ≧90 mmHg or antihypertensive treatment.

Metabolic Syndrome (MBO)

The metabolic syndrome was defined according to recommendations by the National Cholesterol Education Program (NCEP) and the World Health Organization (WHO). The metabolic syndrome as defined by the NCEP was three or more of the following: fasting blood glucose levels ≧5.6 mmol/l (equivalent to plasma glucose levels ≧6.1 mmol/l (Alberti et al, 1998), serum triglycerides ≧1.7 mmol/l, serum HDL <1.0 mmol/l, blood pressure ≧30/85 mmHg or medication, waist girth >102 cm (NCEP, 2001). The WHO definition of the metabolic syndrome was modified as described before (Laaksonen et al 2002), and defined as the presence of hyperinsulinemia (fasting serum insulin concentration in the top 25% of these non-diabetic men), impaired fasting glucose, or diabetes and the presence of at least two of the following: abdominal obesity (WHR >0.90 or BMI ≧30 kg/m2), dyslipidemia (serum triglycerides ≧1.7 mmol/l or serum HDL cholesterol <0.9 mmol/l), or hypertension (blood pressure ≧140/90 mmHg or blood pressure medication) (29). Impaired fasting glucose was defined as a fasting blood glucose 5.6-6.0 mmol/l, equivalent to a plasma glucose of 6.1-6.9 mmol/l (Alberti et al 1998).

Obesity

The body-mass index (BMI) was defined as the ratio of weight in kg to the square of the height in meters. Obesity was defined as BMI of 30 kg/m² or more.

Other Measurements

Age and tobacco smoking were recorded on a self-administered questionnaire checked by an interviewer. Family history of CHD was defined positive if the subject's mother, father or a sibling had a history of either AMI or angina pectoris. Family histories of cerebrovascular stroke, hypertension, diabetes and obesity were defined similarly. These data were collected by a self administered questionnaire.

HDL fractions were separated from fresh serum by combined ultracentrifugation and precipitation. The cholesterol contents of lipoprotein fractions and serum triglycerides were measured enzymatically. Fibrinogen was measured based on the clotting of diluted plasma with excess thrombin.

Serum ferritin was assessed with a commercial double antibody radioimmunoassay (Amersham International, Amersham, UK). Lipoproteins, including high density lipoprotein (HDL) and low density lipoprotein (LDL), were separated from fresh serum samples by ultracentrifugation followed by direct very low density lipoprotein (VLDL) removal and LDL precipitation (Salonen et al 1991). Lipoprotein (a) was assayed by an ELISA method. Cholesterol concentration was then determined enzymically. Serum C-reactive protein was measured by a commercial high-sensitive immunometric assay (Immulite High Sensitivity CR Assay, DPC, Los Angeles).

Genomic DNA Isolation and Quality Testing

High molecular weight genomic DNA samples were extracted from frozen venous whole blood using standard methods and dissolved in standard TE buffer. The quantity and purity of each DNA sample was evaluated by measuring the absorbance at 260 and 280 nm and integrity of isolated DNA samples was evaluated with 0.9% agarose gel electrophoresis and Ethidiumbromide staining. A sample was qualified for genome wide scan (GWS) analysis if A260/A280 ratio was ≧1.7 and average size of isolated DNA was over 20 kb in agarose gel electrophoresis. Before GWS analysis samples were diluted to concentration of 50 ng/μl in reduced EDTA TE buffer (TEKnova).

Genotyping of SNP Markers

Genotyping of SNP markers was performed by using the technology access version of Affymetrix GeneChip® human mapping 100k system. The assay consisted of two arrays, Xba and Hind, which were used to genotype over 126,000 SNP markers from each DNA sample. The assays were performed according to the instructions provided by the manufacturer. A total of 250 ng of genomic DNA was used for each individual assay. DNA sample was digested with either Xba I or Hind III enzyme (New England Biolabs, NEB) in the mixture of NE Buffer 2 (1×; NEB), bovine serum albumin (1×; NEB), and either Xba I or Hind III (0,5 U/ μl; NEB) for 2 h at +37° C. followed by enzyme inactivation for 20 min at +70° C. Xba I or Hind III adapters were then ligated to the digested DNA samples by adding Xba or Hind III adapter (0,25 μM, Affymetrix), T4 DNA ligase buffer (1×; NEB), and T4 DNA ligase (250 U; NEB). Ligation reactions were allowed to proceed for 2 h at +16° C. followed by 20 min incubation at +70° C. Each ligated DNA sample was diluted with 75 μl of molecular biology-grade water (BioWhittaker Molecular Applications/Cambrex).

Diluted ligated DNA samples were subjected to four identical 100 μl volume polymerase chain reactions (PCR) by implementing a 10 μl aliquot of DNA sample with Pfx Amplification Buffer (1×; Invitrogen), PCR Enhancer (1×; Invitrogen), MgSO₄ (1 mM; Invitrogen), dNTP (300μM each; Takara), PCR primer (1μM; Affymetrix), and Pfx Polymerase (0.05 U/μl; Invitrogen). The PCR was allowed to proceed for 3 min at +94° C., followed by 30 cycles of 15 sec at +94° C., 30 sec at +60° C., 60 sec at +68° C., and finally for the final extension for 7 min at +68° C. The performance of the PCR was checked by standard 2% agarose gel electrophoresis in 1×TBE buffer for 1 h at 120V.

PCR products were purified according to Affymetrix manual using MinElute 96 UF PCR Purification kit (Qiagen) by combining all four PCR products of an individual sample into same purification reaction. The purified PCR products were eluted with 40 μl of EB buffer (Qiagen), and the yields of the products were measured at the absorbance 260 nm. A total of 40 μg of each PCR product was then subjected to fragmentation reaction consisting of 0.2 U/μl fragmentation reagent (Affymetrix) in 1×Fragmentation Buffer. Fragmentation reaction was allowed to proceed for 35 min at +37° C. followed by 15 min incubation at +95° C. for enzyme inactivation. Completeness of fragmentation was checked by running an aliquot of each fragmented PCR product in 4% agarose 1×TBE (BMA Reliant precast) for 30-45 min at 120V.

Fragmented PCR products were then labeled using 1×Terminal Deoxinucleotidyl Transferase (TdT) buffer (Affymetrix), GeneChip DNA Labeling Reagent (0.214 mM; Affymetrix), and TdT (1.5 U/μl; Affymetrix) for 2 h at +37° C. followed by 15 min at +95° C. Labeled DNA samples were combined with hybridization buffer consisting of 0.056 M MES solution (Sigma), 5% DMSO (Sigma), 2.5× Denhardt's solution (Sigma), 5.77 mM EDTA (Ambion), 0.115 mg/ml Herring Sperm DNA (Promega), 1×Oligonucleotide Control reagent (Affymetrix), 11.5 μ/ml Human Cot-1 (Invitrogen), 0.0115% Tween-20 (Pierce), and 2.69 M Tetramethyl Ammonium Chloride (Sigma). DNA-hybridization buffer mix was denatured for 10 min at +95° C., cooled on ice for 10 sec and incubated for 2 min at +48° C. prior to hybridization onto corresponding Xba or Hind GeneChip® array. Hybridization was completed at +48° C. for 16-18 h at 60 rpm in an Affymetrix GeneChip Hybridization Oven. Following hybridization, the arrays were stained and washed in GeneChip Fluidics Station 450 according to fluidics station protocol Mapping10Kv1_{—}450 as recommended by the manufacturer. Arrays were scanned with GeneChip 3000 Scanner and the genotype calls for each of the SNP markers on the array were generated using Affymetrix Genotyping Tools (GTT) software. The confidence score in SNP calling algorithm was adjusted to 0.20.

Initial SNP Selection for Statistical Analysis

Prior to the statistical analysis, SNP quality was assessed on the basis of three values: the call rate (CR), minor allele frequency (MAF), and Hardy-Weinberg equilibrium (H-W). The CR is the proportion of samples with successful genotyping result. It does not take into account whether the genotypes are correct or not. The call rate was calculated as: CR=number of samples with successful genotype call/total number of samples. The MAF is the frequency of the allele that is less frequent in the study sample. MAF was calculated as: MAF=min(p, q), where p is frequency of the SNP allele ‘A’ and q is frequency of the SNP allele ‘B’; p=(number of samples with “AA”-genotype+0.5*number of samples with “AB”-genotype)/total number of samples with successful genotype call; q=1−p. SNPs that are homozygous (MAF=0) can not be used in genetic analysis and were thus discarded. H-W equilibrium is tested for controls. The test is based on the standard Chi-square test of goodness of fit. The observed genotype distribution is compared with the expected genotype distribution under H-W equilibrium. For two alleles this distribution is p², 2pq, and q² for genotypes ‘AA’, ‘AB’and ‘BB’, respectively. If the SNP is not in H-W equilibrium it can be due to genotyping error or some unknown population dynamics (e.g. random drift, selection). Only the SNPs that had CR >50%, MAF >1%, and were in H-W equilibrium (Chi-square test statistic <23.93) were used in the statistical analysis.

Statistical Methods

Single SNP Analysis

Differences in allele distributions between cases and controls were screened for all the SNPs in the selected regions. The screening was carried out by using the standard Chi-square independence test with 1 df (allele distribution, 2×2 table). Odds ratio was calculated as ad/bc, where a is the number of minor alleles in cases, b is the number of major alleles in cases, c is the number of minor allele in controls, and d is the number of major alleles in controls. Minor allele was defined as the allele for a given SNP that has smaller frequency than the other allele in the control group.

Haplotype Analysis: HaploRec+HPM

The data set was analyzed with a haplotype pattern mining algorithm HPM software (Toivonen HT et al, 2000). For HPM software genotypes must have phase known i.e. to determine which alleles are coming from the mother and which from the father. Without family data phases must be estimated based on population data. We used HaploRec-program (Eronen L et al, 2004) to estimate the phases. HPM is very fast and can handle a large number of SNPs in a single run

For phase-known data HPM finds all haplotype patterns that are in concordance with the phase configuration. The length of the haplotype patterns can vary. As an example, if there are four SNPs and an individual has alleles A T for the SNP1, C C for the SNP2, C G for the SNP3, and A C for the SNP4 then HPM considers haplotype patterns that are in concordance with estimated phase (done by HaploRec). If the estimated phase is ACGA (from the mother/father) and TCCC (from the father/mother) then HPM considers two patterns (of length 4 SNPs): ACGA and TCCC. A SNP is scored based on the number of times it is included in a haplotype pattern that differs between cases and controls (a threshold Chi-square value can be selected by the user). Significance of the score values is tested based on permutation tests.

Several parameters can be modified in the HPM program including the Chi-square threshold value (−x), the maximum haplotype pattern length (−1), the maximum number of wildcards that can be included in a haplotype pattern (−w), and the number of permutation test in order to estimate the P-value (−p). Wildcards allow gaps in haplotypes. HPM was run with the following parameter settings: haplotype analysis with 5 SNPs (−x9−15−w1−p1000). Haplotype genomic regions that gave P-value less than 0.05 were considered statistically significant.

Multivariate Analyses

Partial associations, adjusted for all independent variables entering the model, were estimated by using the least squares regression analysis for quantitative traits and the binary logistic regression analysis for binary disease outcomes. In the latter, the odds ratios were estimated as the antilogarithms of the partial coefficients. The confidence intervals were from the SPSS for Windows 11.5 software used.

Definition of Terms used in the Haplotype Analysis Results

The term “haplotype genomic region” or “haplotype region” refers to a genomic region that has been found significant in the haplotype analysis (HPM or similar statistical method/program). The haplotype region in this patent is defined as a sub-region of the pre-selected genomic region where for any SNP the permutated P-value is less or equal than 0.05.

The term “haplotype”, as described herein, refers to any combination of alleles e.g. A T C C that is found in the given genetic markers e.g. rs2221511 (A/G), rs4940595 (G/T), rs1522723 (C/T), and rs1395266 (C/T). A defined haplotype gives the name of the genetic markers (dbSNP rs-id for the SNPs) and the alleles. As it is recognized by those skilled in the art the same haplotype can be described differently by determining alleles from different strands e.g. the haplotype rs2221511, rs4940595, rs1522723, and rs1395266 (A T C C) is the same as haplotype rs222151 1, rs4940595, rs1522723, and rs1395266 (T A G G) in which the alleles are determined from the other strand or haplotype rs222151 1, rs4940595, rs1522723, and rs1395266 (A A G G), in which the first allele is determined from the other strand.

It is understood that the at-risk alleles and at-risk haplotypes described in this invention may be associated with other “polymorphic sites” located in genes of this invention. These other polymorphic sites may be either equally useful as genetic markers or even more useful as causative variations explaining the observed association of at-risk alleles and at-risk haplotypes of this invention to any of the diseases considered in this patent.

Findings of the Population Study

SERPIN Genes

In all 250 subjects, of the 59 typed SERPIN-related SNPs, rs720321 (SEQ ID: 136), rs2032225 (SEQ ID: 69), rs1395266 (SEQ ID: 46), rs8091945 (SEQ ID: 150), rs3786335 (SEQ ID: 106), rs10513932 (SEQ ID: 22), rs1015416 (SEQ ID: 2) and rs6103 (SEQ ID: 121) were significantly (p<0.05 in linear-by-linear association) associated with incident AMI, rs1395266 (SEQ ID: 46), rs931850 (SEQ ID: 157), rs1506430 (SEQ ID: 52), rs10513931 (SEQ ID: 21), rs2042729 (SEQ ID: 71), and rs8097354 (SEQ ID: 152) with hypertension.

Table 1 presents the results (as p-values) from haplotype mining pattern analyses for AMI and hypertension. Several both intragenic markers in the SERPINB11 gene and markers flanking the gene were associated with both AMI and HT (table 1). In addition, markers in the SERPINB13 gene and markers flanking the gene were associated with hypertension.

TABLE 1
P-values from HPM analysis of SNP markers in the SERPIN gene region with
respect to AMI and hypertension. AMI-18 and HT-18 are from HPM + HaploRec
with a maximum length of 8 for haplotypes. All other results are from HPM + HaploRec
with a maximum length of 5 for haplotypes

| | SEQ | | | | | | Allele | Allele |
|---|---|---|---|---|---|---|---|---|
| Marker | ID | AMI | AMI-18 | HT | HT-18 | Gene | A | B |
| rs10503081 | 16 | 1 | 1 | 1 | 0.0264 | — | A | G |
| rs1455564 | 51 | 1 | 1 | 1 | 0.0144 | — | C | T |
| rs10503083 | 17 | 1 | 1 | 0.0324 | 0.0064 | — | A | T |
| rs715351 | 134 | 1 | 1 | 0.0148 | 0.0032 | SERPINB13 | A | G |
| rs611263 | 122 | 1 | 1 | 0.0079 | 0.0019 | — | A | G |
| rs1403302 | 47 | 1 | 1 | 0.0043 | 0.0014 | — | C | G |
| rs952857 | 163 | 1 | 1 | 0.0043 | 0.0007 | — | G | T |
| rs1522719 | 54 | 1 | 1 | 0.0071 | 0.0008 | — | C | T |
| rs1581426 | 61 | 1 | 1 | 0.0072 | 0.0012 | SERPINB11 | C | T |
| rs2221511 | 79 | 0.0240 | 0.0748 | 0.0067 | 0.0013 | SERPINB11 | A | G |
| rs4940595 | 117 | 0.0206 | 0.0701 | 0.0057 | 0.0007 | SERPINB11 | G | T |
| rs1522723 | 56 | 0.0178 | 0.0589 | 0.0030 | 0.0006 | SERPINB11 | C | T |
| rs1395266 | 46 | 0.0083 | 0.0201 | 0.0002 | 0.0002 | SERPINB11 | C | T |
| rs931850 | 157 | 0.0090 | 0.0100 | 0 | 0 | — | A | G |
| rs1522722 | 55 | 0.0172 | 0.0120 | 0 | 0.0001 | — | C | T |
| rs10503087 | 19 | 0.0237 | 0.0132 | 0.0014 | 0.0009 | — | A | C |
| rs1701586 | 62 | 0.0309 | 0.0118 | 0.0036 | 0.0011 | — | A | G |
| rs9320028 | 159 | 1 | 0.0128 | 0.0083 | 0.0029 | — | C | T |
| rs1506430 | 52 | 1 | 0.0138 | 1 | 0.0050 | — | A | C |
| rs8091945 | 150 | 1 | 0.0180 | 1 | 0.0080 | — | C | T |
| rs10513933 | 23 | 1 | 0.0298 | 1 | 0.0145 | — | A | C |

Table 2 presents the associations between selected SNPs in the SERPIN gene region and different cardiovascular and metabolic diseases and conditions. The coefficients are from linear step-up regression models testing the entry of all mentioned SNPs (coded as 0. 1, 2 for alphabetically first allele homozygocity, heterozygocity and second allele hozygocity). Statistical significance in dicated by asterices (*** for p<0.001, ** for p<0.01 and * for p<0.05). For instance, for the rs1395266 (SEQ ID: 46) the alleles are C and T, where C is the minor allele. The positive coefficients mean that the allele T carrier status was associated with elevated blood pressure, BMI and subscapular skinfold thickness and increased prevalence of family history of hypertension (in siblings or parents). Of the other SNPs, rs213069 (SEQ ID: 74) appears to be associated most strongly with elevated serum insulin and BMI, rs931850 (SEQ ID: 157) appears to be associated most strongly with markers of CHD and metabolic syndrome and rs1395266 (SEQ ID: 46) with blood pressure, BMI and skinfold thickness. Markers in the SERPIN gene region were also associated with plasma insulin concentration (Table 12).

The rs1395266 (SEQ ID: 46) is a non-synonymous SNP in the coding region of the SERPINB11 gene. It causes a change of isoleucine to threonine in the amino acid postion 293. Of the 231 subjects genotyped for rs1395266 (SEQ ID: 46), 150 were major allele (T) homozygotes, 65 heterozygotes and 16 minor allele (C) homozygotes. Thus, 35.1% were minor allele carriers.

Table 3 shows means and standard deviations of quantitative traits in the genotypes of the SERPINB11 gene rs1395266 (SEQ ID: 46) marker. The marker was significantly associated with both systolic and diastolic blood pressure, and subscapular skinfold thickness (a measure of obesity).

Among all the 231 subjects for whom we had the SERPINB11gene rs1395266 (SEQ ID: 46) genotype, the C allele carriers had lower both systolic and diastolic blood pressure, fasting blood glucose, serum triglyceride and uric acid concentrations and higher serum HDL cholesterol concentration and 24-hour urinary excretion of potassium. The allele C carriers were also leaner, as shown as lower body weight, body-mass index and smaller subscapular and biceps skinfold thicknesses and waist circumference and waist to hip circumference ratio (Table 4).

SERPINBL 11 gene rs1395266 (SEQ ID: 46) allele C carriers also had elevated plasma vitamin C (ascorbic acid) levels and reduced serum C-reactive protein levels (Table 4). This provides evidence for our invention that the allele C carriers are less prone to inflammation. It is also possible that the C-allele carriers have either increased absorption or reduced elimination of arcorbate. Both the serum ferritin concentration (148 vs. 139 microg/L) and the proportion of subjects with elevated serum ferritin levels (200 microg/L or more) (27.5% vs 16.3%, p=0.061) was elevated among the allele C carriers as compared to non-carriers. This could reflect either increased iron absorption or be a consequence of elevated ascorbate availability in the intestinal absorption sites of iron, enhancing the iron absorption. As the carriers had reduced CRP levels, the higher serum ferritin levels are not likely to reflect inflammation, but body iron status.

As shown in table 5, the carrier status of the minor allele C was associated in 163 subjects with an odds ratio for familial HT of 0.23 (95% confidence interval 0.11 to 0.50), Chi-square for the difference in allele frequency was 20.29, p<0.00001). The respective univariate odds ratio for AMI in 231 subjects was 0.45 (95% CI 0.26 to 0.78, Chi-square 8.19, p=0.006).

Among the 231 subjects, the SERPINB11 gene rs1395266 (SEQ ID: 46) allele C carriers had a lower risk of AMI, definite AMI and cerebrovascular stroke and lower prevalence of angina pectoris on effort, any CHD, any hypertension, moderate-to-severe hypertension, type 2 diabetes, the metabolic syndrome and obesity, as compared with the non-carriers (Table 5). The carriers also had less often family history of CHD and hypertension.

In total, our empirical findings show that the SERPMB11 mutation rs1395266 (SEQ ID: 46) is protective against CHD, AMI, HT, MBO and obesity and against exaggerated inflammatory response.

TABLE 2
The strongest associations between SNP markers in the SERPIN gene region and cardiovascular and metabolic outcomes.

| Marker | rs213069 | rs611263 | rs698708 | rs715351 | rs931850 | rs1395266 | rs1944328 | rs2849382 | rs4940605 | rs8094641 | rs8097354 |
|---|---|---|---|---|---|---|---|---|---|---|---|
| SEQ ID | 74 | 122 | 132 | 134 | 157 | 46 | 68 | 96 | 118 | 151 | 152 |
| Incident AMI | | | | | −0.17** | | | | | | −0.26* |
| Angina | | | | −0.07* | | | | | | | |
| pectoris | | | | | | | | | | | |
| Any CHD | | | | | −0.12* | | | | | | |
| CHD family | | | | | −0.17** | | | | | | −0.26* |
| history | | | | | | | | | | | |
| Mean SBP | | | | | | 6.47*** | | | | | |
| Mean DBP | | | | | | 4.00*** | | | | | |
| Family hist. of | | | | | | 0.12* | | | | | |
| hypertension | | | | | | | | | | | |
| Type 2 | | | | −.10*** | | | | | | | |
| diabetes | | | | | | | | | | | |
| Metabolic | | | | | −.13*** | | | | 0.45* | −0.54** | |
| syndrome | | | | | | | | | | | |
| Blood glucose | | 0.51*** | | | | | | | | | |
| Serum insulin | 2.39** | | 1.62* | | | | | | | | |
| HDL | | | | | 0.09** | | | 0.12** | 0.07* | | |
| cholesterol | | | | | | | | | | | |
| Triglycerides | | | | −0.20** | | | | | | | |
| BMI | 0.96* | | | | | 1.38** | −1.07* | | | | |
| Biceps | | | 1.03* | | | | | | | | |
| skinfold | | | | | | | | | | | |
| Subscapular | | | | | | 1.93** | | | | | |
| skinfold | | | | | | | | | | | |

TABLE 3
Associations of marker rs1395266 (SEQ ID: 46), intragenic marker for SERPINB11 gene, with quantitative traits
related to CHD, hypertension, type 2 diabetes and the metabolic syndrome.

| | Minor allele | | | | | | Major allele | | | p-value |
|---|---|---|---|---|---|---|---|---|---|---|
| | C homozygote | | | Heterozygote | | | T homozygote | | | from |
| Quantitative trait | Mean | SD | N | Mean | SD | N | Mean | SD | N | ANOVA* |
| Systolic BP (mmHg) | 124.6 | 10.2 | 16 | 127.2 | 14.3 | 65 | 135.2 | 16.1 | 150 | 0.009 |
| Diastolic BP (mmHg) | 80.0 | 7.4 | 16 | 83.5 | 9.1 | 65 | 87.7 | 10.2 | 150 | 0.003 |
| BMI (kg/m2) | 25.4 | 3.3 | 16 | 25.4 | 3.0 | 65 | 26.9 | 4.0 | 150 | 0.120 |
| Subscapular skinfold (mm) | 11.7 | 5.0 | 16 | 12.8 | 5.4 | 65 | 15.1 | 6.6 | 146 | 0.038 |
| Blood glucose (mmol/L) | 4.55 | 0.56 | 16 | 4.51 | 0.45 | 65 | 4.90 | 1.40 | 149 | 0.260 |
| Plasma insulin (mU/L) | 11.44 | 13.19 | 16 | 9.63 | 4.24 | 65 | 11.59 | 7.03 | 146 | 0.937 |
| Serum LDL cholesterol | 4.14 | 1.66 | 16 | 4.17 | 0.94 | 64 | 4.19 | 1.05 | 149 | 0.876 |
| (mmol/L) | | | | | | | | | | |
| Serum HDL cholesterol | 1.32 | 0.32 | 16 | 1.36 | 0.38 | 65 | 1.26 | 0.27 | 150 | 0.475 |
| (mmol/L) | | | | | | | | | | |
| Serum apolipoprotein AI (g/L) | 1.33 | 0.21 | 16 | 1.37 | 0.29 | 64 | 1.31 | 0.24 | 143 | 0.805 |
| Serum triglycerides (mmol/L) | 1.07 | 0.55 | 16 | 1.24 | 0.65 | 64 | 1.40 | 0.80 | 150 | 0.104 |
| Serum apolipoprotein (a) U/L | 330.0 | 440.9 | 15 | 272.3 | 260.7 | 64 | 251.6 | 310.3 | 142 | 0.348 |
| Plasma fibrinogen (g/L) | 3.09 | 0.49 | 16 | 2.93 | 0.48 | 60 | 3.11 | 0.56 | 138 | 0.908 |
| Plasma C-reactive protein | 1.02 | 0.96 | 16 | 1.59 | 1.93 | 65 | 2.98 | 7.87 | 142 | 0.245 |
| (mg/L) | | | | | | | | | | |

*Statistical significance of the linear component of variation across the three genotypes.

TABLE 4
Distributions (means, standard deviations) of selected
cardiovascular and metabolic risk and other factors in
SERPINB11 rs1395266 (SEQ ID: 46) allele C carriers
and non-carriers.

| | SERPINB11 | | Non-carriers | | |
|---|---|---|---|---|---|
| | C allele | | (n = 150) | | p-value |
| Measurement | Mean | SD | Mean | SD | for |
| Mean systolic BP | 126.6 | 13.6 | 135.2 | 16.1 | <0.001 |
| (mmHg) | | | | | |
| Mean diastolic BP | 82.8 | 8.9 | 87.7 | 10.2 | <0.001 |
| (mmHg) | | | | | |
| Fasting blood glucose | 4.5 | 0.47 | 4.9 | 1.40 | 0.003 |
| Mean waist | 85.7 | 8.0 | 90.2 | 9.6 | 0.001 |
| circumference | | | | | |
| Waist-to-hip | 0.928 | 0.052 | 0.954 | 0.101 | 0.021 |
| circumference | | | | | |
| Body mass index (kg/m²) | 25.4 | 3.0 | 26.9 | 4.0 | 0.002 |
| Body weight (kg) | 74.7 | 10.1 | 78.9 | 12.7 | 0.007 |
| Subscapular skinfold | 12.6 | 5.3 | 15.1 | 6.6 | 0.002 |
| Skinfold thickness at the | 4.2 | 1.9 | 5.6 | 5.1 | 0.004 |
| Serum C-reactive protein | 1.48 | 1.79 | 2.98 | 7.87 | 0.030 |
| 24-h urinary potassium | 70 | 26 | 61 | 18 | 0.030 |
| Serum HDL cholesterol | 1.35 | 0.37 | 1.26 | 0.27 | 0.043 |
| Serum triglyceride | 1.21 | 0.63 | 1.40 | 0.80 | 0.055 |
| Serum uric acid | 0.323 | 0.055 | 0.342 | 0.064 | 0.029 |
| Plasma ascorbic acid | 9.1 | 4.6 | 7.4 | 4.3 | 0.009 |

* From t-tests assuming unequal variances.

TABLE 5
Odds ratios for the association between selected cardiovascular
and metabolic conditions and SERPINB11 rs1395266
(C allele carrier vs. non-carrier, n = 231).

| | | | | | p-value |
|---|---|---|---|---|---|
| | SERPINB11 | | Non-carriers | | for |
| | C allele | | (n = 150) | | differ- |
| Measurement | Mean | SD | Mean | SD | ence* |
| Mean systolic BP | 126.6 | 13.6 | 135.2 | 16.1 | <0.001 |
| (mmHg) | | | | | |
| Mean diastolic BP | 82.8 | 8.9 | 87.7 | 10.2 | <0.001 |
| (mmHg) | | | | | |
| Fasting blood glucose | 4.5 | 0.47 | 4.9 | 1.40 | 0.003 |
| Mean waist | 85.7 | 8.0 | 90.2 | 9.6 | 0.001 |
| circumference | | | | | |
| Waist-to-hip | 0.928 | 0.052 | 0.954 | 0.101 | 0.021 |
| circumference | | | | | |
| Body mass index (kg/m²) | 25.4 | 3.0 | 26.9 | 4.0 | 0.002 |
| Body weight (kg) | 74.7 | 10.1 | 78.9 | 12.7 | 0.007 |
| Subscapular skinfold | 12.6 | 5.3 | 15.1 | 6.6 | 0.002 |
| Skinfold thickness at the | 4.2 | 1.9 | 5.6 | 5.1 | 0.004 |
| Serum C-reactive protein | 1.48 | 1.79 | 2.98 | 7.87 | 0.030 |
| 24-h urinary potassium | 70 | 26 | 61 | 18 | 0.030 |
| Serum HDL cholesterol | 1.35 | 0.37 | 1.26 | 0.27 | 0.043 |
| Serum triglyceride | 1.21 | 0.63 | 1.40 | 0.80 | 0.055 |
| Serum uric acid | 0.323 | 0.055 | 0.342 | 0.064 | 0.029 |
| Plasma ascorbic acid | 9.1 | 4.6 | 7.4 | 4.3 | 0.009 |

*Waist >94/82 cm, fB-glucose >5.6 mmol/L
** The criteria include waist-to-hip circumference ratio (see above)
*** Two-sided, based on Fisher's exact test

Associations of SERPINB11 Gene Haplotypes with AMI

Haplotypes were estimated for 1 Mb region (59Mb - 60Mb) around the SERPINB11 gene. Typed SNP markers in this region are presented in Table 1. Estimation was done with the HaploRec program. Haplotype 1: rs1395266 (SEQ ID: 46) (C/T), rs931850 (SEQ ID: 157) (A/G), rs1522722 (SEQ ID: 55) (C/T), rs1701586 (SEQ ID: 62) (A/G), rs9320028 (SEQ ID: 159) (C/T), rs1506430 (SEQ ID: 52) (A/C), and rs8091945 (C/T) (SEQ ID: 150) defining the haplotype TACGTAT (or nucleotides from the complementary strand). The table gives a Chi-square value of 14.70 (p<0.001) and an odds ratio of 2.11 with 95% CI: 1.44 to 3.10 for the haplotype.

| | | Case chromosomes | Control chromosomes |
|---|---|---|---|
| Haplotype 1 | Yes | 167 | 124 |
| | No | 67 | 105 |

Associations of SERPINB11 Gene Haplotypes with HT

Analysis revealed two haplotypes that were highly significantly associated with familial hypertension (n=163). Haplotype 1: rs2221511 (SEQ ID: 79) (A/G), rs4940595 (SEQ ID: 117) (G/T), rs1522723 (SEQ ID: 56) (C/T), and rs1395266 (SEQ ID: 46) (C/T) defining the haplotype ATCC (or nucleotides from the complementary strand). Below is a 2 by 2 table showing the number of chromosomes in the familial hypertension cases and controls according to the presence of haplotype. The Chi-square value for association is 22.42 (p<0.00001) and the odds ratio 0.20 with 95% confidence interval of 0.10 to 0.41 for the ATCC haplotype.

| | | Case chromosomes | Control chromosomes |
|---|---|---|---|
| Haplotype 1 | Yes | 11 | 42 |
| | No | 143 | 110 |

Haplotype 2: rs1395266 (SEQ ID: 46) (C/T), rs931850 (SEQ ID: 157) (A/G), and rs1522722 (SEQ ID: 55) (C/T) defining the haplotype T A C (or nucleotides from the complementary strand). Below is a 2 by 2 table showing how many haplotypes found in our sample populations are Haplotype 2 (yes) and how many are something else (no). The Chi-square value is 23.72 (p<0.00001) and odds ratio 4.23 with 95% CI 2.31 to 7.78 for the TAC haplotype

| | | Case chromosomes | Control chromosomes |
|---|---|---|---|
| Haplotype 2 | Yes | 137 | 97 |
| | No | 17 | 51 |

SPINK Genes

Table 6 presents the results (as p-values) from haplotype mining pattern analyses for AMI and type 2 diabetes. The SNP marker rs10515605 (SEQ ID: 32) is positioned in the SPINK5L3 gene. In the KIHD data set, there were 178 allele G homozygotes, 38 AG heterozygotes and no allele A homozygotes for the rs10515605 (SEQ ID: 32) SNP (n=216). The heterozygous AG genotype was associated with a 9.1-fold (95% CI 3.4 to 24.3, p<0.000001) risk of AMI as compared with the wild type GG homozygotes.

TABLE 6
P-values from HPM analysis of SNP markers in the SPINK
gene family region with respect to AMI.

| Marker | SEQ ID | AMI | Gene | Allele A | Allele B |
|---|---|---|---|---|---|
| rs724726 | 140 | 1 | — | A | C |
| rs1029884 | 4 | 1 | SPINK5L2 | C | G |
| rs10491344 | 13 | 1 | — | C | T |
| rs1432689 | 49 | 0.016 | — | A | G |
| rs1023714 | 3 | 0.0047 | LOC402232 | A | T |
| rs2400502 | 89 | 0.0011 | — | A | G |
| rs2400503 | 90 | 0.0005 | — | C | T |
| rs10515605 | 32 | <0.0001 | SPINK5L3 | A | G |
| rs7709159 | 145 | 0.0007 | — | C | T |
| rs3749690 | 105 | 0.0058 | ECG2 | C | T |
| rs10515609 | 33 | 0.0132 | — | C | T |
| rs10515610 | 34 | 0.0364 | — | C | T |
| rs1363707 | 43 | 1 | — | C | T |
| rs10515613 | 35 | 1 | FBXO38 | A | G |

All results are from HPM + HaploRec with a maximum length of 5 for haplotypes.

The haplotype analysis revealed one haplotype that had a strong association with AMI:

Haplotype 1: rs2400503 (SEQ ID: 90) (C/T), rs10515605 (SEQ ID: 32) (AIG), and rs7709159 (SEQ ID: 145) (C/T), defining the haplotype CAC (or nucleotides from the complementary strand). The chi-square for association was 24.90 (p<0.000001) and odds ratio 11.85 with 95% CI 3.56 to 39.40 for the CAC haplotype.

| | | AMI Case | AMI Control |
|---|---|---|---|
| | | chromosomes | chromosomes |
| Haplotype 1 | Yes | 31 | 3 |
| | No | 184 | 211 |

The associations of the SPINKL5L3 rs10515605 (SEQ ID: 32) genotype with different cardiovascular outocomes are presented in table 7. There were very strong associations with the risk of incident (new) acute myocardial infarction as well as with cardiovascular and coronary mortality. The marker was also associated with hypertension, the risk of stroke and angina pectoris (Table 7).

The table 8 shows the occurrence of fatal, nonfatal or no AMI during the follow-up according to SPINK5L3 rs10515605 (SEQ ID: 32) SNP genotypes. The allele A carrier status (AG heterozygocity) was associated with 34.5-fold risk of fatal AMI (death due to CHD) and 20.0-fold risk of CHD death, as compared to no CHD death. Thus, the marker predicted fatal AMI stronger than non-fatal AMI, but predicted both strongly and significantly.

The means and standard deviations of selected cardiovascular and metabolic risk and other factors in SPINK5L3 rs10515605 A allele carriers and non-carriers are presented in table 9. Also other markers in the SPINK5L3 gene were associated with a number of these traits (Table 12).

TABLE 7
Odds ratios for the association between selected cardiovascular
and metabolic conditions and SPINKL5L3 rs10515605
(SEQ ID: 32) genotype (A allele carrier vs. non-carrier, n = 216).

| | Odds | | |
|---|---|---|---|
| Disease condition | ratio | 95% CI | P* |
| Incident acute myocardial infarction | 9.06 | 3.38, 24.31 | <0.000001 |
| Incident definite AMI | 2.84 | 1.39, 5.82 | 0.007 |
| Prevalent angina pectoris on effort | 3.04 | 1.35, 6.85 | 0.013 |
| Any prevalent CHD | 3.46 | 1.65, 7.34 | 0.001 |
| Family history of CHD | 9.06 | 3.38, 24.31 | <0.000001 |
| Incident cerebrovascular stroke | 4.07 | 1.04, 15.94 | 0.053 |
| Cardiovascular death | 17.09 | 7.18, 40.68 | <0.000001 |
| CHD death | 18.75 | 7.75, 45.39 | <0.000001 |
| Prevalent hypertension | 2.41 | 1.16, 5.01 | 0.020 |
| (140/90 or more or drug) | | | |
| Prevalent moderate to severe HT | 4.51 | 2.16, 9.43 | <0.0001 |
| (160/95 or more) | | | |

*Two-sided, based on Fisher's exact test

TABLE 8
Association between the genotype of the rs10515605 (SEQ ID: 32)
marker (in the SPINK5L3 gene) and occurrence of AMI and
death due to CHD during the follow-up. Chi-square 64.93,
df 2, p < 0.001, odds ratio for the AG genotype vs GG
genotype 34.5, 95% CI 11.11 to 111.11 for CHD death vs
no AMI, 20.0, 95% CI 8.26 to 47.62 for CHD death vs
other and 9.1, 95% CI 3.38 to 24.39 for AMI vs no AMI.

| | Genotype of rs10515605 | | |
|---|---|---|---|
| | (SEQ ID: 32) marker | | |
| Outcome | AG | GG | Total |
| No AMI | 5 | 102 | 107 |
| Nonfatal AMI | 9 | 62 | 71 |
| Fatal AMI | 22 | 13 | 35 |
| Total | 36 | 177 | 213 |

The SPINKL5L3 rs10515605 (SEQ ID: 32) A allele carriers had significantly elevated plasma fibrinogen, serum apolipoprotein B, serum triglyceride levels and reduced serum HDL-to-LDL cholesterol ratio, serum HDL cholesterol concentrations and 24-hour urinary excretion of potassium, as compared with the non-carriers (Table 9). Also their blood pressures tended to be higher and their fasting blood glucose was higher, if tested with a t-test assuming equal variances, otherwise there was a non-significant treand.

TABLE 9
Distributions (means, standard deviations) of selected
cardiovascular and metabolic risk and other factors in
SPINKL5L3 rs10515605 (SEQ ID: 32) A allele
carriers and non-carriers, n = 216).

| | | | | | p-value |
|---|---|---|---|---|---|
| | SPINKL5L3 | | | | for |
| | rs10515605 | | | | differ- |
| | (SEQ ID: 32) | | | | ence |
| | A allele | | | | (t-tests |
| | carriers | | Non-carriers | | for |
| | (n = 38) | | (n = 177) | | unequal |
| Measurement | Mean | SD | Mean | SD | variances) |
| Mean systolic BP (mmHg) | 135.1 | 19.2 | 130.6 | 14.1 | 0.174 |
| Mean diastolic BP (mmHg) | 86.3 | 10.5 | 85.4 | 9.3 | 0.648 |
| Fasting blood glucose | 5.0 | 1.45 | 4.6 | 0.88 | 0.133 |
| (mmol/L) | | | | | (0.039) |
| Plasma fibrinogen (g/L) | 3.32 | 0.56 | 2.96 | 0.49 | 0.001 |
| 24-h urinary potassium | 58 | 17 | 67 | 22 | 0.032 |
| excretion (mmol) | | | | | |
| Serum HDL cholesterol | 1.26 | 0.30 | 1.30 | 0.30 | 0.417 |
| (mmol/L) | | | | | |
| Serum apolipoprotein B | 1.13 | 0.20 | 1.05 | 0.25 | 0.037 |
| (mg/L) | | | | | |
| Serum HDL-to-LDL ratio | 0.29 | 0.09 | 0.35 | 0.16 | 0.004 |
| Serum triglyceride | 1.35 | 0.72 | 1.30 | 0.70 | 0.720 |
| concentration (mmol/L) | | | | | |

Interaction Between SERPINB11, SPINKL5L3 and other Genes

The SPINKL5L3 gene rs10515605 (SEQ ID: 32) A allele carrier status elevated the risk of AMI extremely strongly (OR 45.0, p<0.001) among 126 SERPINB11gene rs1395266 (SEQ ID: 46) TT homozygotes and very strongly among 59 heterozygotes (OR 15.3, p=0.004), while the association was weak in those 14 who were minor allele CC homozygotes (OR 2.0, p=1.000). This finding was confirmed by pooling all allele C carriers: the SPINKL5L3 gene had a stronger effect on AMI risk in SERPINB11 rs1395266 (SEQ ID: 46) major allele T carriers than in the minor allele C carriers.

TABLE 10
Odds ratio for AMI, associated with selected markers, among carriers
and noncarriers of SPINKL5L3 rs10515605 A allele

| | SPINKL5L3 | | | | | |
|---|---|---|---|---|---|---|
| | rs10515605 A | | | | | |
| | allele carriers | | | Non-carriers | | |
| | | 95% | | | 95% | P- |
| Marker | OR | CI | P* | OR | CI | value |
| rs551681 | 0.007 | n.a. | 0.001 | 0.55 | 0.23, | 0.206 |
| SEQ ID: | | | | | 1.36 | |
| 166 | | | | | | |
| rs10495576 | 0.054 | 0.004, | 0.050 | 0.52 | 0.24, | 0.127 |
| SEQ ID: | | 0.781 | | | 1.16 | |
| 167 | | | | | | |
| rs778160 | 0.097 | 0.010, | 0.076 | 0.63 | 0.29, | 0.261 |
| SEQ ID: | | 0.956 | | | 1.36 | |
| 168 | | | | | | |

*Two-sided from Fisher's exact test.

TABLE 11
Odds ratio for AMI, associated with selected markers, among carriers
and noncarriers of SERPINB11 rs1395266 (SEQ ID: 46) allele C

| | SERPINB11 C | | | | | |
|---|---|---|---|---|---|---|
| | allele carriers | | | Non-carriers | | |
| | | 95% | | | 95% | P- |
| Marker | OR | CI | P* | OR | CI | value |
| rs10515605 | 8.60 | 1.67, | 0.005 | 45 | n.a. | <.001 |
| SEQ ID: 32 | | 44.2 | | | | |
| rs6733858 | 2.16 | 0.60, | 0.319 | 7.61 | 2.18, | <.001 |
| SEQ ID: | | 7.80 | | | 26.6 | |
| 169 | | | | | | |
| rs1357540 | 0.76 | 0.31, | 0.648 | 0.31 | 0.16, | 0.001 |
| SEQ ID: | | 1.88 | | | 0.62 | |
| 170 | | | | | | |

SPOCK Genes

TABLE 12
P-values from HPM analysis of SNP markers in the SPOCK gene
region with respect to AMI and hypertension.

| | SEQ | | | | | |
|---|---|---|---|---|---|---|
| Marker | ID | AMI | HT | Gene | Allele A | Allele B |
| rs739699 | 143 | 0.0587 | 1 | SPOCK | C | T |
| rs1229718 | 41 | 0.0393 | 1 | SPOCK | A | G |
| rs10515495 | 24 | 0.0040 | 0.1208 | SPOCK | A | G |
| rs4976445 | 119 | 0.0001 | 0.0903 | SPOCK | A | G |
| rs10491335 | 11 | 0.0001 | 0.1001 | — | A | G |
| rs10491336 | 12 | 0.0002 | 0.1085 | — | A | G |
| rs6878439 | 131 | <0.0001 | 0.1045 | — | A | G |
| rs1034664 | 5 | 0.0015 | 1 | KLHL3 | C | G |
| rs2860269 | 98 | 0.0087 | 1 | KLHL3 | C | T |
| rs2349034 | 88 | 0.0283 | 1 | KLHL3 | A | T |
| rs2905598 | 99 | 0.0321 | 1 | KLHL3 | C | T |
| rs2905610 | 100 | 1 | 1 | KLHL3 | C | G |
| rs2905617 | 101 | 1 | 1 | KLHL3 | C | T |
| rs2967806 | 102 | 1 | 1 | KLHL3 | C | T |
| rs10515496 | 25 | 1 | 1 | KLHL3 | A | G |
| rs700613 | 133 | 1 | 1 | TTID | G | T |
| rs9327807 | 162 | 1 | 1 | C5orf5 | A | G |
| rs1381726 | 45 | 1 | 1 | C5orf5 | A | T |
| rs10515497 | 26 | 1 | 1 | — | C | T |
| rs10515498 | 27 | 1 | 1 | — | C | T |
| rs10515500 | 29 | 1 | 1 | CDC25C | C | G |
| rs10515499 | 28 | 1 | 1 | JMJD1B | C | T |
| rs757649 | 144 | 1 | 1 | JMJD1B | A | C |
| rs219278 | 78 | 1 | 1 | — | A | G |
| rs154079 | 57 | 1 | 1 | ETF1 | C | T |
| rs256015 | 92 | 1 | 1 | HSPA9B | C | T |

All results are from HPM + HaploRec with a maximum length of 5 for haplotypes

Table 12 presents the results (as p-values) from haplotype mining pattern analyses for AMI and hypertension.

The SNP marker rs4976445 (SEQ ID: 119) is positioned in an intron of the SPOCK gene in chromosome 5. In our data, the marker was significantly associated with the risk of AMI, definite AMI and with the family history of CHD (Table 13).

TABLE 13
Odds ratios for the association between selected CHD-related
outcomes and SPOCK rs4976445 (SEQ ID: 119) genotype
(C allele carrier vs. non-carrier, n = 232).

| Disease condition | Odds ratio | 95% CI | P* |
|---|---|---|---|
| Incident acute myocardial infarction | 2.24 | 1.27, 3.95 | 0.007 |
| Incident definite AMI | 2.73 | 1.52, 4.90 | 0.001 |
| Any prevalent CHD | 1.75 | 0.91, 3.36 | 0.117 |
| Family history of CHD | 2.24 | 1.27, 3.95 | 0.007 |

*Two-sided, based on Fisher's exact test

The allele C carriers of rs4976445 (SEQ ID: 119) had significantly higher mean diastolic BP, serum apolipoprotein B concentration and lower HLD-to-LDL cholesterol ratio than the non-carriers (Table 14). Also other markers in the SPOCK gene were associated with blood pressure, serum LDL and HDL cholesterol, apolipoprotein B and apolipoprotein(a) (Tables 15 and 16).

TABLE 14
Distributions (means, standard deviations) of selected
cardiovascular and metabolic risk and other factors in
SPOCK rs4976445 (SEQ ID: 119) genotypes (C allele
carrier vs. non-carrier, n = 232).

| | | | | | p-value |
|---|---|---|---|---|---|
| | SPOCK | | | | for |
| | rs4976445 | | | | differ- |
| | (SEQ ID: 119) | | | | ence |
| | C allele | | | | (t-tests |
| | carriers | | Non-carriers | | for |
| | (n = 74) | | (n = 160) | | unequal |
| Measurement | Mean | SD | Mean | SD | variances) |
| Mean systolic BP (mmHg) | 132.4 | 13.9 | 131.5 | 16.3 | 0.653 |
| Mean diastolic BP (mmHg) | 87.9 | 9.2 | 84.9 | 10.2 | 0.024 |
| Serum HDL cholesterol | 1.24 | 0.26 | 1.31 | 0.32 | 0.098 |
| (mmol/L) | | | | | |
| Serum apolipoprotein B | 1.12 | 0.25 | 1.04 | 0.24 | 0.022 |
| (mg/L) | | | | | |
| Serum HDL-to-LDL ratio | 0.31 | 0.12 | 0.35 | 0.16 | 0.042 |
| Serum triglyceride | 1.36 | 0.62 | 1.30 | 0.76 | 0.506 |
| concentration (mmol/L) | | | | | |

The SNP marker rs6826647 (SEQ ID: 128) is positioned at a distance of 3,174 bp from the 3′ of the SPOCK3 gene. Table 16 shows the associations of the genotypes of this marker with quantitative traits related to hypertension, obesity and the metabolic syndrome. The marker was associated with systolic and diastolic blood pressure, body-mass index, and fasting blood glucose and fasting plasma insulin concentrations.

The marker rs4860001 (SEQ ID: 116) in the SPOCK3 gene was associated with plasma concentration of fibrinogen (Table 18).

TABLE 15
Standardized regression coefficients for associations of intragenic
SNP markers in the regions of SPOCK and SPOCK3 genes with
lipids in step-up multivariate linear regression analyses.

| | | | | | Apolipo- | Apolipo- |
|---|---|---|---|---|---|---|
| | | | LDL | HDL/LDL | protein | protein |
| Marker | SEQ ID | Closest Gene/s | cholesterol | ratio | B | (a) |
| rs28063 | 94 | SPOCK/ | −0.22** | | −0.18** | |
| | | LOC391834 | | | | |
| rs10491299 | 10 | SPOCK | | −0.175** | 0.18** | |
| rs6876032 | 130 | SPOCK | | | | −0.16** |
| rs10517908 | 37 | SPOCK3 | −0.18** | | | |

Table 16 showns means and standard deviations of quantitative traits in genotypes of rs6826647 (SEQ ID: 128), flanking the SPOCK3 gene. The marker was significantly associated with blood pressure, fasting blood glucose and almost significantly associated with BMI.

TABLE 16
Associations of marker rs6826647 (SEQ ID: 128), flanking the SPOCK3 gene,
with quantitative traits related to CHD, hypertension and the metabolic syndrome.

| | Minor allele | | | | | | Major allele | | | p-value |
|---|---|---|---|---|---|---|---|---|---|---|
| | homozygote | | | Heterozygote | | | homozygote | | | from |
| Quantitative trait | Mean | SD | N | Mean | SD | N | Mean | SD | N | ANOVA* |
| Systolic BP (mmHg) | 134.6 | 17.7 | 55 | 131.7 | 15.1 | 129 | 129.3 | 14.2 | 64 | 0.067 |
| Diastolic BP (mmHg) | 89.00 | 11.8 | 55 | 85.9 | 9.2 | 129 | 82.6 | 8.6 | 64 | 0.000 |
| BMI (kg/m2) | 26.9 | 5.0 | 55 | 26.5 | 3.2 | 129 | 25.6 | 3.0 | 64 | 0.052 |
| Subscapular skinfold (mm) | 14.3 | 8.0 | 52 | 14.7 | 5.9 | 128 | 12.8 | 4.9 | 64 | 0.191 |
| Blood glucose (mmol/L) | 5.04 | 1.62 | 55 | 4.72 | 1.09 | 128 | 4.58 | 0.54 | 64 | 0.029 |
| Plasma insulin (mU/L) | 12.12 | 8.45 | 55 | 11.57 | 7.28 | 127 | 9.28 | 4.13 | 62 | 0.028 |
| Serum LDL cholesterol | 4.35 | 1.16 | 54 | 4.09 | 1.00 | 128 | 4.22 | 1.14 | 64 | 0.514 |
| (mmol/L) | | | | | | | | | | |
| Serum HDL cholesterol | 1.29 | 0.27 | 55 | 1.26 | 0.28 | 129 | 1.38 | 0.36 | 64 | 0.105 |
| (mmol/L) | | | | | | | | | | |
| Serum apolipoprotein AI (g/L) | 1.33 | 0.22 | 52 | 1.30 | 0.23 | 124 | 1.41 | 0.29 | 63 | 0.113 |
| Serum triglycerides (mmol/L) | 1.34 | 0.76 | 55 | 1.37 | 0.77 | 128 | 1.21 | 0.64 | 64 | 0.351 |
| Serum apolipoprotein (a) U/L | 309.7 | 359.1 | 52 | 242.3 | 278.2 | 126 | 272.1 | 325.4 | 60 | 0.522 |
| Plasma fibrinogen (g/L) | 3.04 | 0.54 | 53 | 3.07 | 0.56 | 120 | 3.01 | 0.49 | 58 | 0.797 |
| Plasma C-reactive protein | 1.87 | 2.18 | 52 | 2.77 | 8.19 | 126 | 1.98 | 2.80 | 62 | 0.922 |
| (mg/L) | | | | | | | | | | |

*Statistical significance of the linear component of variation across the three genotypes.

Multivariate and other Summary Analyses Concerning All Genes

Table 17 presents standardized linear regression coefficients from multivariate regression analyses. Several markers in or flanking SERPIN, SPINK and SPOCK genes were associated with blood pressure and plasma fibrinogen. A marker in the SERPIN region was associated with plasma insulin concentration, a proxi measure of insulin sensitivity.

TABLE 17
Standardized regression coefficients for associations of SNP markers in the regions
of SERPIN, SPINK and SPOCK genes with quantitative traits related to the metabolic
syndrome in step-up multivariate linear regression analyses

| | SEQ | | Plasma | Systolic | Diastolic | Plasma |
|---|---|---|---|---|---|---|
| Marker | ID | Gene | insulin | BP | BP | fibrinogen |
| rs213069 | 74 | SERPINB8/C18orf20 | 0.20** | | | |
| rs931850 | 157 | SERPINB11/ | | −0.20** | −0.21*** | |
| | | SERPINB7 | | | | |
| rs10515605 | 32 | SPINK5L3^{#} | | | | −0.18** |
| rs1860933 | 65 | LOC391839/SPINK5 | | −0.19** | | |
| rs1434651 | 50 | SPOCK^{#} | | | 0.17** | |
| rs4860001 | 116 | SPOCK3^{#} | | | | −0.17** |
| rs2703843 | 93 | SPOCK3^{#} | | | −0.21*** | |
| rs1352968 | 42 | TLL1/SPOCK3 | | | 0.17** | |

^{#}intragenic SNP.
**denotes p < 0.01,
***p < 0.001.

Table 18 shows results of univariate analysis concerning associations between all 306 SNP markers in the SERPIN, SPINK, SPOCK gene regions and four disease outcomes: AMI, HT and CHD death.

Tables 19 and 20 present findings from step-up multivariate binary logistic regression analyses, in which a total of 306 SNP markers from the SERPIN, SPINK, SPOCK gene regions were tested for entry (PIN=0.01). They summarize the strongest associations of SNP markers in these gene regions with the risk of binary disease outcomes examined. In summary, markers in or flanking both the SERPIN, SPINK, SPOCK genes predicted the development of AMI and/or coronary death, or were associated with prevalent CHD (Table 19. Also, markers in or flanking both SERPIN, SPINK, SPOCK genes were associated with hypertension or family history of hypertension, and diabetes or the metabolic syndrome or the family history of diabetes. Markers in SPINK, SPOCK gene regions were associated with either obesity of family history of obesity.

TABLE 15
Standardized regression coefficients for associations of intragenic
SNP markers in the regions of SPOCK and SPOCK3 genes with
lipids in step-up multivariate linear regression analyses.

| | SPOCK | | | | p-value |
|---|---|---|---|---|---|
| | rs4976445 | | | | for |
| | (SEQ ID: | | | | difference |
| | 119) C | | | | (t- |
| | allele | | | | tests |
| | carriers | | Non-carriers | | for |
| | (n = 74) | | (n = 160) | | unequal |
| Measurement | Mean | SD | Mean | SD | variances) |
| Mean systolic BP (mmHg) | 132.4 | 13.9 | 131.5 | 16.3 | 0.653 |
| Mean diastolic BP (mmHg) | 87.9 | 9.2 | 84.9 | 10.2 | 0.024 |
| Serum HDL cholesterol | 1.24 | 0.26 | 1.31 | 0.32 | 0.098 |
| (mmol/L) | | | | | |
| Serum apolipoprotein B | 1.12 | 0.25 | 1.04 | 0.24 | 0.022 |
| (mg/L) | | | | | |
| Serum HDL-to-LDL ratio | 0.31 | 0.12 | 0.35 | 0.16 | 0.042 |
| Serum triglyceride | 1.36 | 0.62 | 1.30 | 0.76 | 0.506 |
| concentration (mmol/L) | | | | | |

The SNP marker rs6826647 (SEQ ID: 128) is positioned at a distance of 3,174 bp from the 3'of the SPOCK3 gene. Table 16 shows the associations of the genotyof this marker with quantitative traits related to hypertension, obesity and the metabolic syndrome. The marker was associated with systolix and diastolic blood pressure, body-mass index, and fasting blood glucose and fasting plasma insulin concentrations. The marker rs4860001 (SEQ ID: 116) in the SPOCK3 gene was associated with plasma concentration of fibrinogen (Table 18).

TABLE 18
Results from univariate single SNP analysis (2by2 table with odds ratio OR)
for AMI, hypertension (HT) and CHD deaths.

| Identification | | AMI | | HT | | CHD deaths | | Annotation information | | |
|---|---|---|---|---|---|---|---|---|---|---|
| Marker | SEQ ID | P-value | OR | P-value | OR | P-value | OR | Gene | CHR | Minor allele |
| rs739699 | 143 | 0.1827 | 1.4892 | 0.4554 | 1.3230 | 0.7596 | 0.8771 | SPOCK | 5 | C |
| rs1229718 | 41 | 0.7957 | 0.9531 | 0.3463 | 1.2422 | 0.8414 | 0.9510 | SPOCK | 5 | A |
| rs10515495 | 24 | 0.0085 | 2.1488 | 0.2924 | 1.4471 | 0.4722 | 1.3489 | SPOCK | 5 | A |
| rs4976445 | 119 | 0.0020 | 2.1905 | 0.0467 | 1.7907 | 0.4725 | 1.2654 | SPOCK | 5 | G |
| rs10491335 | 11 | 0.2094 | 1.3331 | 0.3040 | 1.3315 | 0.0036 | 2.1948 | — | 5 | A |
| rs10491336 | 12 | 0.0422 | 0.6896 | 0.5722 | 0.8788 | 0.0051 | 0.4740 | — | 5 | A |
| rs6878439 | 131 | 0.0052 | 2.2031 | 0.3390 | 1.4010 | 0.0027 | 3.0532 | — | 5 | A |
| rs1034664 | 5 | 0.9151 | 1.0234 | 0.3607 | 0.7754 | 0.0681 | 0.5331 | KLHL3 | 5 | G |
| rs2860269 | 98 | 0.0553 | 1.5080 | 0.0571 | 1.6270 | 0.0025 | 2.1767 | KLHL3 | 5 | C |
| rs2349034 | 88 | 0.1353 | 1.3460 | 0.2065 | 1.3704 | 0.0017 | 2.6373 | KLHL3 | 5 | T |
| rs2905598 | 99 | 0.1853 | 1.3473 | 0.2333 | 1.3736 | 0.0027 | 2.2807 | KLHL3 | 5 | C |
| rs2905610 | 100 | 0.1403 | 1.4226 | 0.1375 | 1.5324 | 0.0073 | 2.2093 | KLHL3 | 5 | G |
| rs2905617 | 101 | 0.0779 | 1.4614 | 0.0551 | 1.6355 | 0.0023 | 2.2072 | KLHL3 | 5 | T |
| rs2967806 | 102 | 0.0912 | 1.4323 | 0.0665 | 1.5891 | 0.0021 | 2.2000 | KLHL3 | 5 | C |
| rs10515496 | 25 | 0.2516 | 0.7790 | 0.7795 | 0.9246 | 0.0248 | 0.4554 | KLHL3 | 5 | G |
| rs700613 | 133 | 0.0960 | 1.4159 | 0.3165 | 1.2857 | 0.0046 | 2.0885 | TTID | 5 | G |
| rs9327807 | 162 | 0.1485 | 0.7216 | 0.7932 | 0.9276 | 0.0287 | 0.4312 | C5orf5 | 5 | G |
| rs1381726 | 45 | 0.5190 | 0.8118 | 0.4647 | 0.7544 | 0.6087 | 1.2353 | C5orf5 | 5 | A |
| rs10515497 | 26 | 0.3688 | 1.1803 | 0.2857 | 0.7858 | 0.8460 | 1.0500 | — | 5 | T |
| rs10515498 | 27 | 0.7800 | 1.0812 | 0.7047 | 1.1366 | 0.1763 | 1.5824 | — | 5 | T |
| rs10515500 | 29 | 0.7898 | 0.9429 | 0.4208 | 1.2507 | 0.9211 | 0.9702 | CDC25C | 5 | C |
| rs10515499 | 28 | 0.4606 | 0.8472 | 0.2729 | 1.3515 | 0.4201 | 0.7647 | JMJD1B | 5 | C |
| rs757649 | 144 | 0.8598 | 1.0400 | 0.1270 | 0.6526 | 0.6482 | 1.1438 | JMJD1B | 5 | A |
| rs219278 | 78 | 0.6939 | 1.0758 | 0.3947 | 0.8232 | 0.3946 | 1.2351 | — | 5 | G |
| rs154079 | 57 | 0.5877 | 0.7438 | 0.7275 | 1.2390 | 0.6056 | 1.4061 | ETF1 | 5 | C |
| rs256015 | 92 | 0.6113 | 0.7714 | 0.5419 | 1.4361 | 0.7838 | 1.1958 | HSPA9B | 5 | T |
| rs2032866 | 70 | 0.5528 | 0.7899 | 0.1783 | 2.0921 | 0.1997 | 0.3969 | — | 5 | A |
| rs7727019 | 146 | 0.4326 | 0.7271 | 0.1011 | 2.5987 | 0.2209 | 0.4124 | — | 5 | T |
| rs953310 | 164 | 0.5528 | 0.7899 | 0.1783 | 2.0921 | 0.1997 | 0.3969 | — | 5 | C |
| rs1423003 | 48 | 0.8073 | 1.0500 | 0.9519 | 0.9849 | 0.2512 | 0.7184 | SPINK5 | 5 | G |
| rs2303064 | 84 | 0.4596 | 1.2003 | 0.9137 | 0.9670 | 0.0144 | 2.0345 | SPINK5 | 5 | A |
| rs1862446 | 66 | 0.9205 | 1.0201 | 0.8509 | 0.9535 | 0.2611 | 0.7233 | SPINK5 | 5 | C |
| rs2303066 | 85 | 0.5276 | 1.1208 | 0.8056 | 0.9460 | 0.4299 | 1.2132 | SPINK5 | 5 | C |
| rs2303069 | 86 | 0.7999 | 0.8973 | 0.1503 | 2.3543 | 0.3035 | 0.4701 | SPINK5 | 5 | C |
| rs3815740 | 107 | 0.1656 | 1.4877 | 0.6003 | 0.8324 | 0.0005 | 2.9268 | SPINK5 | 5 | C |
| rs2287770 | 83 | 0.3019 | 1.3452 | 0.3260 | 0.7044 | 0.0005 | 2.9268 | SPINK5 | 5 | C |
| rs10515603 | 31 | 0.1613 | 1.3031 | 0.8468 | 0.9554 | 0.4016 | 1.2340 | SPINK5 | 5 | A |
| rs10515602 | 30 | 0.1689 | 1.2940 | 0.8487 | 0.9563 | 0.4055 | 1.2315 | SPINK5 | 5 | C |
| rs6873836 | 129 | 0.1951 | 1.2780 | 0.8117 | 0.9448 | 0.4275 | 1.2257 | SPINK5 | 5 | A |
| rs724726 | 140 | 0.4303 | 0.8658 | 0.7363 | 0.9272 | 0.5403 | 0.8571 | — | 5 | C |
| rs1029884 | 4 | 0.1727 | 0.7790 | 0.8978 | 1.0294 | 0.3780 | 0.8015 | SPINK5L2 | 5 | C |
| rs10491344 | 13 | 0.3609 | 1.1839 | 0.5748 | 1.1377 | 0.5003 | 0.8438 | — | 5 | C |
| rs1432689 | 49 | 0.3145 | 1.1992 | 0.5725 | 1.1343 | 0.5383 | 0.8597 | — | 5 | A |
| rs1023714 | 3 | 0.8528 | 1.0713 | 0.6587 | 1.2160 | 0.6286 | 0.7668 | LOC402232 | 5 | T |
| rs2400502 | 89 | 0.6859 | 1.1809 | 0.3722 | 1.5672 | 0.7697 | 0.8316 | — | 5 | G |
| rs2400503 | 90 | 0.5378 | 0.7755 | 0.9778 | 1.0136 | 0.2639 | 0.4430 | — | 5 | T |
| rs10515605 | 32 | 0.0000 | 7.6098 | 0.0879 | 2.1270 | 0.0000 | 11.1964 | SPINK5L3 | 5 | A |
| rs7709159 | 145 | 0.9887 | 0.9917 | 0.0134 | 0.0000 | 0.9530 | 1.0474 | — | 5 | T |
| rs3749690 | 105 | 0.2721 | 0.7836 | 0.0563 | 0.5953 | 0.0298 | 0.4298 | ECG2 | 5 | T |
| rs10515609 | 33 | 0.8579 | 0.9437 | 0.2268 | 1.6914 | 0.4131 | 1.3862 | — | 5 | C |
| rs10515610 | 34 | 0.7121 | 0.9172 | 0.6636 | 0.8857 | 0.0802 | 0.5046 | — | 5 | C |
| rs1363707 | 43 | 0.5430 | 0.8793 | 0.1530 | 0.6927 | 0.1089 | 0.5832 | — | 5 | T |
| rs10515613 | 35 | 0.7038 | 1.1449 | 0.5900 | 1.3202 | 0.1263 | 0.3387 | FBXO38 | 5 | G |
| rs10503083 | 17 | 0.1140 | 0.4000 | 0.2361 | 0.4456 | 0.3346 | 0.3788 | — | 18 | T |
| rs715351 | 134 | 0.6802 | 1.0887 | 0.3780 | 1.2491 | 0.8190 | 1.0650 | SERPINB13 | 18 | A |
| rs611263 | 122 | 0.4142 | 1.2218 | 0.1767 | 1.5012 | 0.7927 | 1.0918 | — | 18 | G |
| rs1403302 | 47 | 0.1777 | 1.2750 | 0.1806 | 1.3499 | 0.3087 | 1.2863 | — | 18 | C |
| rs952857 | 163 | 0.5688 | 1.1143 | 0.1098 | 1.4551 | 0.7273 | 0.9124 | — | 18 | T |
| rs1522719 | 54 | 0.4633 | 1.1547 | 0.0839 | 1.5206 | 0.8886 | 0.9636 | — | 18 | T |
| rs1581426 | 61 | 0.5669 | 1.1156 | 0.1005 | 1.4725 | 0.7488 | 0.9192 | SERPINB11 | 18 | T |
| rs2221511 | 79 | 0.2969 | 0.7847 | 0.9572 | 1.0153 | 0.1558 | 0.6022 | SERPINB11 | 18 | G |
| rs4940595 | 117 | 0.5688 | 1.1143 | 0.1098 | 1.4551 | 0.7273 | 0.9124 | SERPINB11 | 18 | G |
| rs1522723 | 56 | 0.5847 | 1.1122 | 0.1202 | 1.4481 | 0.7142 | 0.9035 | SERPINB11 | 18 | T |
| rs1395266 | 46 | 0.0020 | 0.4869 | 0.0000 | 0.2317 | 0.2375 | 0.6717 | SERPINB11 | 18 | C |
| rs931850 | 157 | 0.0018 | 0.4958 | 0.0000 | 0.2714 | 0.1807 | 0.6389 | — | 18 | G |

dbSNP_rs_ID: SNP identification number in NCBI dbSNP database build 124
Seq_ID: Sequence identification number
Gene: Gene positioned in the physical position pointed by the SNP according to NCBI Human Genome Build 35
Position db 124: Basepair position according to NCBI Human Genome Build 35
Allele A and B: Alternate SNP alleles or its complementary nucleotide for the given SNP
CHR: Chromosome

TABLE 19
Odds ratio and statistical significance for the strongest associations of SNP
markers in the chromosomal regions of SERPIN,
SPINK, SPOCK genes with CHD related outcomes
in step-up multivariate logistic regression analyses

| | | | | Definite | | Prevalent |
|---|---|---|---|---|---|---|
| Marker | SEQ ID | Closest Gene/s | AMI | AMI | CHD death | CHD |
| rs1455564 | 51 | SERPINB5/SERPINB12 | | | | 2.18** |
| rs1395266 | 46 | SERPINB11^{#} | 2.25** | | | |
| rs1015416 | 2 | SERPINB2^{#} | | 0.43** | | |
| rs7730218 | 147 | SPINK1/SCGB3A2 | | | | 2.88*** |
| rs3815740 | 107 | SPINK5^{#} | | | 0.21** | |
| rs10515605 | 32 | SPINK5L3^{#} | | | 0.10*** | |
| rs715423 | 135 | SPOCK^{#} | | | 7.29*** | |
| rs4976445 | 119 | SPOCK^{#} | | 2.61*** | | 2.37** |
| rs10515495 | 24 | SPOCK^{#} | 0.28*** | | 0.10*** | |
| rs6878439 | 131 | SPOCK/KLHL3 | 0.35*** | | 0.11*** | |
| rs10491335 | 11 | SPOCK/KLHL3 | | | 0.22** | |
| rs4691246 | 114 | TLL1/SPOCK3 | 0.50** | | | |
| rs1373557 | 44 | TLL1/SPOCK3 | | | 9.43** | |
| rs10517876 | 36 | TLL1/SPOCK3 | | | | 0.40** |
| rs2153987 | 77 | NXT1, ZNF336, NAPB, CSTL1, CST11 | 0.34** | | | |
| Cases (n) | | | 123 | 75 | 40 | 82 |

^{#}intragenic SNP

TABLE 20
Odds ratio and statistical significance for the strongest associations of SNP markers
in the chromosomal regions of SERPIN, SPINK, SPOCK and CST genes with
metabolic syndrome related outcomes in step-up multivariate logistic regression analyses.

| | | | | Mild to | Moderate | | | |
|---|---|---|---|---|---|---|---|---|
| | | | | severe | to severe | Family | Obesity | Family |
| | | | Metabolic | HT (BP | HT (BP | history | (BMI 30 or | history of |
| Marker | SEQ ID | Closest Gene/s | syndrome* | 140/90) | 165/95) | of HT | more) | obesity |
| rs1455564 | 51 | SERPINB5/SERPINB12 | | | | | | |
| rs611263 | 122 | SERPINB13/SERPINB4 | 3.43** | | | | | |
| rs931850 | 157 | SERPINB11/SERPINB7 | 0.21** | 0.37*** | 0.37** | 0.53** | | |
| rs8097354 | 152 | SERPINB10^{#} | | 0.19* | 0.06** | | | |
| rs8094641 | 151 | SERPINB8/C18orf20 | 0.29** | | | | | |
| rs10515605 | 32 | SPINK5L3^{#} | | | 0.33** | | | |
| rs3749690 | 105 | SPINK5L3/ECG2 | | | | | | |
| rs10515610 | 34 | SPINK5L3, ECG2/FBX038 | | | | | | 2.15** |
| rs1859346 | 64 | SPOCK^{#} | | | | 0.55** | | |
| rs1560929 | 60 | SPOCK^{#} | | | | | | 1.68** |
| rs2060428 | 72 | SPOCK^{#} | 0.33** | | | | 0.36** | |
| rs739699 | 143 | SPOCK^{#} | | | | | | |
| rs4691246 | 114 | TLL1/SPOCK3 | | | 0.39*** | | | |
| rs388102 | 111 | TLL1/SPOCK3 | | | | | | 2.74** |
| rs904246 | 154 | TLL1/SPOCK3 | | | | | 0.23** | |
| rs10517927 | 38 | SPOCK3^{#} | 0.22*** | | | | 0.38** | |
| rs6137917 | 126 | ZNF336/NAPB, CSTL1, CST11 | | | 0.27* | | 0.19** | |
| Cases (n) | | | 27 | 121 | 79 | 123 | 29 | 141 |
| *The metabolic syndrome was defined as the presence of hyperinsulinemia (fasting serum insulin concentration in the top 25% of non-diabetic men), impaired fasting glucose, or diabetes and the presence of at least two of the following: abdominal obesity (waist circumference >102 cm), dyslipidemia (serum triglycerides ≧1.7 mmol/l or serum HDL cholesterol <0.9 mmol/l), or hypertension (blood pressure ≧140/90 mmHg or blood pressure medication). | | | | | | | | |
| # Impaired fasting glucose was defined as a fasting blood glucose 5.6-6.0 mmol/l, equivalent to a plasma glucose of 6.1-6.9 mmol/l. Diabetes was defined as fasting blood glucose concentration ≧6.0 mmol/l equivalent to plasma glucose ≧7.0 mmol/l) or a clinical diagnosis of diabetes with either dietary, oral or insulin treatment. | | | | | | | | |

^{#}intragenic SNP

### EXAMPLE 2

Partial Sequencing of the SPINK5L3 Gene

The coding regions of 29 DNA samples were sequenced in order to find sequence variants from the SPINK5L3 gene. Twelve samples were from patients with family history of AMI, hypertension and T2D and a medical history of at least two of the diseases, the controls (n=17) were free of all these three diseases and had no family history of any of them. The SPINK5L3 gene consists of six exons, of which four were coding exons and two 5′ untranslated exons. By sequencing we identified a variant form of the human SPINK5L3 gene. This variant gene encodes a substitution of amino acid alanine (wild type) to serine (variant form) in the 62^{th} amino acid of the polypeptide.

The PCR (polymerase chain reaction) amplification was conducted in a 20 μL volume. The reaction mixture contained 10 ng human genomic DNA (extracted from peripheral blood), 1×PCR Buffer (QIAGEN), 100 μM of each of the nucleotides (dATP, dCTP, dGTP, dTTP, Finnzymes), 20 pmol of the PCR primer pairs (table 1) and 1 unit of the DNA-polymerase (HotStartTaq, QIAGEN). The PCR was conducted with the PTC 220 DYAD thermocycler (MJ Research) where the program was: 94° C. 7 min, 35× (94° C. 45 s, annealing temperature 30 s, 72° C. 1 min) 72° C. 5 min and hold at 4° C. Depending on the PCR amplicon the annealing temperature varied between 51° C. and 65° C. Prior the sequencing reaction, the PCR amplicons were purified by mixing 10 μL of the PCR product with 2 μL of ExoSAP-IT (USB Corporation) and incubated 30 minutes at 37° C., 15 minutes at 80° C. and stored at 4° C.

The sequencing reactions were made by using the BigDye Terminator Cycle Sequencing v2.0 Ready Reactions with AmpliTaq DNA Polymerase, FS DNA Sequencing Kit (Applied Biosystems) and contained 4 μL RR MIX, 2 μL PCR product, 2 μL sequencing primer (2 pmol/μL) and 2 μL water. The sequencing primers are shown in table 2. Cycle sequencing was conducted with PTC 220 DYAD thermocycler (MJ Research) where the program was: 25 cycles; 10 sec at 96° C., 5 sec at 50° C. and 4 min at 60° C. and hold at 4° C.

TABLE 21

The nucleotide sequences of the PCR primer
pairs (in 5′ to 3′ direction) that were used
to amplify the SPINK5L3 5′ untranslated (UTR)
exons 1 and 2 and the SPINK5L3 coding exons
1 to 4.

| | Nucleotide sequence | |
|---|---|---|
| PCR primer name | of the primers | SEQ ID |
| 5′ UTR exons 1-2 F | ATCTTTCTCCACAACCAAGGTC | 176 |
| 5′ UTR exons 1-2 R | CAAAATTGTAGCAGGGGCATA | 177 |
| exon 1 F | CAGGTCAGTATAAATCACCAG | 178 |
| exon 1 R | CTCCACCATGTAGAAAACAAG | 179 |
| exon 2 F | TTACCCCAATTGCAGTGAAGAG | 180 |
| exon 2 R | AGCCACTGTGCCTGACTTTCC | 181 |
| exon 3 F | GTCCTCTAGAAGTTCACAAACA | 182 |
| exon 3 R | CATCAGTAAAACCCAATTCC | 183 |
| exon 4 F | CTGGTATTTCTATGTTGAATGG | 184 |
| exon 4 R | TGGGGTAGGGTTTAATTCTG | 185 |

F = forward primer
R = reverse primer

Dye terminator removal and sequencing reaction clean up was made with ethanol/EDTA precipitation. More specifically, 10 μL of the sequencing product, 2.5 μL of 125 mM EDTA and 30 μL of 100% ethanol were mixed in a sample plate and incubated at room temperature for 15 min and centrifuged 3000×g for 30 min (Centrifuge 5810, Eppendorf). Ethanol was removed by inverting the sample plate and centrifuging it at 185×g for 1 min. Next 30 μL of 70% ethanol was added and the samples were centrifuged 1650×g for 15 min after which the ethanol was removed as described above. The precipitate was then dissolved to 10μL Hi-Di Formamide (Applied Biosystems), transferred to 96-well plate (MicroAmp, Optical 96-Well Reaction Plate, Applied Biosystems) and sequenced with the ABI PRISM 3100 Genetic Analyzer (Applied Biosystems) and analyzed with the Sequencing Analysis Software (Applied Biosystems) and the SeqManll program (DNASTAR).

TABLE 22

The nucleotide sequences of the primers (in 5′
to 3′ direction) used in the sequencing of the
SPINK5L3 gene. The coding exons 1 and 2 were
sequenced in both directions. The coding exons
3 and 4 and the 5′ UTR exons 1 and 2 were
sequenced only in one direction.

| Target exon and | Nucleotide sequence | |
|---|---|---|
| primer orientation | of the primers | SEQ ID |
| 5′UTR exons 1-2 R | CAAAATTGTAGCAGGGGCATA | 177 |
| Exon 1 F | CAGGTCAGTATAAATCACCAG | 178 |
| Exon 1 R | CTCCACCATGTAGAAAACAAG | 179 |
| Exon 2 F | TTACCCCAATTGCAGTGAAGAG | 180 |
| Exon 2 R | AGCCACTGTGCCTGACTTTCC | 181 |
| Exon 3 F | GTCCTCTAGAAGTTCACAAACA | 182 |
| Exon 4 R | TGGGGTAGGGTTTAATTCTG | 185 |

F = forward primer
R = reverse primer

SPINK5L3 gene sequencing results

Three known and two novel sequence variations were found in sequencing. Known sequence variations were dbSNP (rs6149288, SEQ ID: 127), which is an insertion of 41 base pairs in the second untranslated exon of the gene. The insertion was present in two samples. From two samples we found the SNP marker rs2304030 (SEQ ID: 87), which is C>T substitution 6 base pairs after the second untranslated exon. We found the SNP marker rs1549886 (SEQ ID: 59), the G>C substitution 68 base pairs before the exon 1 from two samples.

From one sample we found a novel mutation from the Kazal domain region of exon 3. This mutation changes the amino acid Alanine to Serine at codon 62 (SEQ ID:174) and this variation has not been reported earlier in dbSNP. In addition we found a SNPfrom exon 4 coding region (SEQ ID: 175), this SNP did not change the Asp amino acid of the codon 94 and this variant has not been reported earlier.

### EXAMPLE 3

Replication in the East Finnish Population

Study Population

The “North Savo Health Survey” was carried out in October to December, 2003. The survey was targeted to all households in the municipalities of Kuopio, Karttula, Lapinlahti, Leppävirta, Maaninka, Rautalampi, Siilinjärvi, Suonenjoki, Tervo, Vehmersalmi, and Vesanto. The number of households was about 70,000 and the number of people over 18 years old was about 200,000. A letter was sent to each household containing three personal and one common questionnaire. The three oldest persons who were at least 18 years of age in the household were asked to fill in the personal questionnaire and one of them to fill in the common family data questionnaire, and return them in the same single return envelope. Only persons, who gave the consent to obtain their hospital records and who provided their personal identification code, were asked to return the questionnaire. The “North Savo Project” included the collection of disease, family, drug response and contact information. By the end of 2004, 17,100 participants were surveyed. The North Savo Survey date were used to identify probands (cases with a trait or disease).

In the second phase, the “SOHFA” project, patients with T2D and T2D-free controls were examined. SOHFA is a contractual study, in which the University of Kuopio is the contractee. “GEDINO” (Genetics of type 2 diabetes in North Savo) is a similar contractual project, in which the T2D cases and controls were collected by using a newspaper advertisement. Information was collected also on hypertension and obesity.

Definition of Cases and Controls

The subject was treated as a hypertensive case, if (s)he either has previously diagnosted hypertension or both high blood pressure (SBP at least 160 or DBP at least 95) and family history of hypertension. 140 subjects fulfilled the above criteria. Normotensive controls (182 subjects) have neither diagnosis of hypertension, elevated BP or family history of hypertension.

The subject was treated as an obesity case, if (s)he either has previous diagnosis of obesity or BMI of 30 kg/m2 or more. Obesity controls have no previous diagnosis of obesity and BMI 25 or less and no family history of obesity. 108 cases and 83 controls fulfilled the above criteria.

Genotyping with Illumina's Sentrix HumanHap300

DNA isolation of cases and controls were done as described in example 1 .The whole-genome genotyping of the DNA samples was performed by using Illumina's Sentrix HumanHap300 BeadChips and Infinium II genotyping assay. The HumanHap300 BeadChip contained over 317,000 tag SNPs markers derived from the International HapMap Project. TagSNPs are loci that can serve as proxies for many other SNPs. The use of tagSNPs greatly improves the power of association studies as only a subset of loci needs to be genotyped while maintaining the same information and power as if one had genotyped a larger number of SNPs.

The Infinium II genotyping with the HumanHap300 BeadChipassays was performed according to the “Single-Sample BeadChip Manual process” described in detail in “Infinium™ II Assay System Manual” provided by Illumina (San Diego, Calif., USA). Briefly, 750 ng of genomic DNA from a sample was subjected to whole-genome amplification. The amplified DNA was fragmented, precipitated and resuspended to hybridization buffer. The resuspended sample was heat denatured and then applied to one Sentrix HumanHap300 beadchip. After overnight hybridization mis- and non-hybridized DNA was washed away from the BeadChip and allele-specific single-base extension of the oligos on the BeadChip was performed in a Tecan GenePaint rack, using labeled deoxynucleotides and the captured DNA as a template. After staining of the extended DNA, the BeadChips were washed and scanned with the BeadArray Reader (Illumina) and genotypes from samples were called by using the BeadStudio software (Illumina).

SNPs Tested

245 SNPs in Illumina HumanHap 300K that are located at genes: C18orf20, C5orf5, CDC25C, ETF1, FBXO38, HSPA9B, JMJD1B, KLHL3, LOC128820, LOC391834, LOC391839, LOC402232, MYOT, NAPB, NXT1, SCGB3A2, SERPINB1, SERPINB10, SERPINB11, SERPINB12, SERPINB13, SERPINB2, SERPINB3, SERPINB4, SERPINB5, SERPINB7, SERPINB8, SERPINB9, SPINK1, SPINK5, SPINK5L2, SPINK5L3, SPINK7, SPOCK1, SPOCK2, SPOCK3, TLL1, and ZNF336 were genotyped for case and control subjects.

Results from the EF Replication Study

In Table 23 are listed SNPs that had significant association (P<0.05) with hypertension.

TABLE 23
Significant associations between SNPs and hypertension (P-value < 0.05) from
single point analysis base on 115 cases and 142 controls (part of subjects, typed first).

| Marker | SEQ ID | Gene | P-value | OR^{a} | Allele^{b} | Chromosome | Position |
|---|---|---|---|---|---|---|---|
| rs4557401 | 203 | C5orf5 | 0.00371 | 0.518792 | G | 5 | 137311711 |
| rs7709766 | 206 | KLHL3 | 0.003963 | 0.541015 | G | 5 | 137093223 |
| rs2303067 | 192 | SPINK5 | 0.004303 | 1.666022 | G | 5 | 147461148 |
| rs4519913 | 202 | SPINK5 | 0.004303 | 1.666022 | A | 5 | 147452004 |
| rs7724165 | 207 | SPINK5 | 0.004303 | 1.666022 | A | 5 | 147445445 |
| rs3777134 | 199 | SPINK5 | 0.014828 | 0.632307 | G | 5 | 147478212 |
| rs3764930 | 198 | SPINK5 | 0.016412 | 0.642517 | G | 5 | 147485309 |
| rs1432691 | 189 | SPINK5L3 | 0.016514 | 0 | G | 5 | 147645401 |
| rs4513684 | 201 | SPINK5L3 | 0.016514 | 0 | C | 5 | 147632278 |
| rs4472254 | 200 | SPINK5 | 0.030689 | 0.670914 | A | 5 | 147433830 |
| rs9949690 | 208 | C18orf20 | 0.038231 | 0.5328 | A | 18 | 59950667 |

^{a}Odds ratio,
^{b}Minor allele

Table 24 lists SNPs that had significant association (P<0.05) with obesity.

TABLE 24
Significant associations between SNPs and obesity (P-value < 0.05) from
single point analysis base on 108 cases and 83 controls.

| Marker | SEQ ID | Gene | P-value | OR^{a} | Allele^{b} | Chromosome | Position |
|---|---|---|---|---|---|---|---|
| rs10517903 | 187 | SPOCK3 | 0.007396 | 1.787541 | C | 4 | 168086584 |
| rs1460060 | 190 | TLL1 | 0.014115 | 0.534373 | C | 4 | 167173034 |
| rs318477 | 195 | SERPINB9 | 0.026983 | 1.620317 | G | 6 | 2832651 |
| rs3744942 | 197 | SERPINB5 | 0.032196 | 1.595 | G | 18 | 59295342 |
| rs6567356 | 204 | SERPINB5 | 0.03866 | 1.580067 | G | 18 | 59296946 |
| rs13110600 | 188 | TLL1 | 0.042251 | 0.636287 | C | 4 | 167167664 |
| rs3192243 | 196 | SPOCK | 0.047227 | 1.50831 | A | 5 | 136340950 |
| rs10517901 | 186 | SPOCK3 | 0.049685 | 0.657534 | C | 4 | 168049635 |
| rs2305593 | 193 | SPOCK3 | 0.049685 | 0.657534 | A | 4 | 168050365 |
| rs7691894 | 205 | SPOCK3 | 0.049685 | 0.657534 | G | 4 | 168038394 |

^{a}Odds ratio,
^{b}Minor allele

We also repeated the statistical analyses concerning hypertension in 178 Eastern Finnish subjects. The material included 72 male and 68 female cases and 90 male and 92 female controls. The larger sample size allowed us to analyze men and women separately (Table 25).

TABLE 25
Associations of SNPs in or close to selected proteolytic system related genes with
hypertension, based on single point (univariate) analysis of 140 cases and 182 controls,
separately for men and women.

| | | | | | | | | Relation of |
|---|---|---|---|---|---|---|---|---|
| | | P-men | P-women | | | | | SNP to |
| Marker | P-all | (n = 162) | (n = 160) | Alleles | Chrom | Position | Gene | gene |
| RS922146 | 0.517206 | 0.713246 | 0.20753 | ‘A/G’ | 4 | 167155959 | TLL1 | intron |
| RS1460062 | 0.098663 | 0.023507 | 0.957084 | ‘C/T’ | 4 | 167161889 | TLL1 | intron |
| RS13110600 | 0.086202 | 0.027942 | 0.827119 | ‘G/T’ | 4 | 167167664 | TLL1 | intron |
| RS1460060 | 0.103571 | 0.004332 | 0.504421 | ‘G/T’ | 4 | 167173034 | TLL1 | intron |
| RS1018139 | 0.767872 | 0.486239 | 0.952536 | ‘C/T’ | 4 | 167205606 | TLL1 | intron |
| RS7654070 | 0.894372 | 0.975791 | 0.846761 | ‘A/G’ | 4 | 167212265 | TLL1 | intron |
| RS1393851 | 0.138359 | 0.003105 | 0.313961 | ‘C/T’ | 4 | 167220490 | TLL1 | intron |
| RS1057377 | 0.625254 | 0.808228 | 0.603736 | ‘A/G’ | 4 | 168030803 | SPOCK3 | reference |
| RS7681289 | 0.231123 | 0.14512 | 0.773315 | ‘C/T’ | 4 | 168034068 | SPOCK3 | intron |
| RS7691894 | 0.841632 | 0.818604 | 0.678333 | ‘A/G’ | 4 | 168038394 | SPOCK3 | intron |
| RS10517901 | 0.883717 | 0.818604 | 0.732053 | ‘A/C’ | 4 | 168049635 | SPOCK3 | intron |
| RS2305593 | 0.883717 | 0.818604 | 0.732053 | ‘C/T’ | 4 | 168050365 | SPOCK3 | intron |
| RS7689440 | 0.675815 | 0.60697 | 0.899195 | ‘C/T’ | 4 | 168059943 | SPOCK3 | intron |
| RS897511 | 0.307187 | 0.279338 | 0.794466 | ‘A/C’ | 4 | 168070391 | SPOCK3 | intron |
| RS6817936 | 0.25521 | 0.378792 | 0.434908 | ‘C/T’ | 4 | 168072978 | SPOCK3 | intron |
| RS7698457 | 0.661796 | 0.032799 | 0.133085 | ‘A/G’ | 4 | 168074672 | SPOCK3 | intron |
| RS10517903 | 0.070939 | 0.906717 | 0.009268 | ‘A/C’ | 4 | 168086584 | SPOCK3 | intron |
| RS4241627 | 0.753999 | 0.667307 | 0.86361 | ‘C/T’ | 4 | 168099328 | SPOCK3 | intron |
| RS1551491 | 0.208275 | 0.237934 | 0.002536 | ‘C/T’ | 4 | 168101866 | SPOCK3 | intron |
| RS954722 | 0.349152 | 0.419933 | 0.667901 | ‘A/G’ | 4 | 168119159 | SPOCK3 | intron |
| RS921856 | 0.409484 | 0.456214 | 0.729754 | ‘C/T’ | 4 | 168119946 | SPOCK3 | intron |
| RS7669089 | 0.453495 | 0.017676 | 0.201454 | ‘A/C’ | 4 | 168128190 | SPOCK3 | intron |
| RS6834387 | 0.507252 | 0.113054 | 0.011531 | ‘C/T’ | 4 | 168146164 | SPOCK3 | intron |
| RS897514 | 0.568782 | 0.051097 | 0.239154 | ‘C/T’ | 4 | 168151603 | SPOCK3 | intron |
| RS7660401 | 0.442593 | 0.466495 | 0.823925 | ‘C/T’ | 4 | 168155645 | SPOCK3 | intron |
| RS7440269 | 0.340406 | 0.154729 | 0.959937 | ‘A/G’ | 4 | 168163937 | SPOCK3 | intron |
| RS10517910 | 0.409484 | 0.456214 | 0.729754 | ‘A/G’ | 4 | 168171644 | SPOCK3 | intron |
| RS13123001 | 0.160824 | 0.777565 | 0.082336 | ‘A/G’ | 4 | 168174629 | SPOCK3 | intron |
| RS9997140 | 0.006829 | 0.156994 | 0.016525 | ‘C/T’ | 4 | 168176208 | SPOCK3 | intron |
| RS10517907 | 0.456967 | 0.305494 | 0.97338 | ‘C/T’ | 4 | 168189336 | SPOCK3 | intron |
| RS6846723 | 0.464228 | 0.117822 | 0.008965 | ‘C/T’ | 4 | 168212437 | SPOCK3 | intron |
| RS10020692 | 0.567776 | 0.318797 | 0.087497 | ‘C/T’ | 4 | 168218338 | SPOCK3 | intron |
| RS10014833 | 0.215417 | 0.2669 | 0.003697 | ‘G/T’ | 4 | 168221992 | SPOCK3 | intron |
| RS7682265 | 0.253428 | 0.422716 | 0.410439 | ‘C/T’ | 4 | 168241573 | SPOCK3 | intron |
| RS4241629 | 0.522365 | 0.977176 | 0.437586 | ‘A/G’ | 4 | 168252717 | SPOCK3 | intron |
| RS6848156 | 0.503578 | 0.562722 | 0.806085 | ‘G/T’ | 4 | 168298994 | SPOCK3 | intron |
| RS10517920 | 0.846726 | 0.987714 | 0.594113 | ‘A/G’ | 4 | 168307239 | SPOCK3 | intron |
| RS6553490 | 0.52025 | 0.531175 | 0.89362 | ‘A/G’ | 4 | 168307740 | SPOCK3 | intron |
| RS10517919 | 0.468684 | 0.911655 | 0.373993 | ‘C/T’ | 4 | 168337178 | SPOCK3 | intron |
| RS6829378 | 0.12625 | 0.618278 | 0.097063 | ‘C/T’ | 4 | 168370419 | SPOCK3 | intron |
| RS1427635 | 0.040888 | 0.328972 | 0.056995 | ‘C/T’ | 4 | 168374080 | SPOCK3 | intron |
| RS12505847 | 0.263464 | 0.727332 | 0.228255 | ‘A/C’ | 4 | 168387799 | SPOCK3 | intron |
| RS11939479 | 0.789702 | 0.432565 | 0.192316 | ‘A/G’ | 4 | 168397424 | SPOCK3 | intron |
| RS4557401 | 0.042441 | 0.222324 | 0.082002 | ‘C/T’ | 5 | 137311711 | C5orf5 | intron |
| RS2058311 | 0.467887 | 0.569214 | 0.69598 | ‘C/T’ | 5 | 137326640 | C5orf5 | intron |
| RS4835662 | 0.462933 | 0.436489 | 0.821073 | ‘C/T’ | 5 | 137326789 | C5orf5 | intron |
| RS4835748 | 0.095047 | 0.861418 | 0.009766 | ‘C/T’ | 5 | 137361288 | C5orf5 | intron |
| RS9327807 | 0.895575 | 0.141152 | 0.102898 | ‘C/T’ | 5 | 137378377 | C5orf5 | intron |
| RS4472254 | 0.040829 | 0.144305 | 0.134417 | ‘G/T’ | 5 | 147433830 | SPINK5 | intron |
| RS7724165 | 0.013842 | 0.077708 | 0.074378 | ‘C/T’ | 5 | 147445445 | SPINK5 | intron |
| RS4519913 | 0.013842 | 0.077708 | 0.074378 | ‘A/G’ | 5 | 147452004 | SPINK5 | intron |
| RS2303064 | 0.279151 | 0.548721 | 0.408557 | ‘A/G’ | 5 | 147460273 | SPINK5 | reference |
| RS2303067 | 0.016718 | 0.077708 | 0.091274 | ‘A/G’ | 5 | 147461148 | SPINK5 | reference |
| RS2287770 | 0.478288 | 0.565463 | 0.653276 | ‘C/T’ | 5 | 147471411 | SPINK5 | intron |
| RS3777134 | 0.010805 | 0.023508 | 0.186807 | ‘C/T’ | 5 | 147478212 | SPINK5 | reference |
| RS1422993 | 0.136335 | 0.177238 | 0.375014 | ‘G/T’ | 5 | 147484013 | SPINK5 | intron |
| RS3764930 | 0.025411 | 0.048606 | 0.225004 | ‘C/T’ | 5 | 147485309 | SPINK5 | intron |
| RS4513684 | 0.019978 | 0.119609 | 0.083576 | ‘A/C’ | 5 | 147632278 | SPINK5L3 | intron |
| RS1432691 | 0.019978 | 0.119609 | 0.083576 | ‘A/G’ | 5 | 147645401 | SPINK5L3 | intron |
| RS12655663 | 0.367978 | 0.958833 | 0.236282 | ‘C/T’ | 5 | 147645416 | SPINK5L3 | intron |
| RS318477 | 0.86591 | 0.414139 | 0.274094 | ‘A/G’ | 6 | 2832651 | SERPINB9 | mrna-utr |
| RS1052886 | 0.235661 | 0.122713 | 0.944484 | ‘C/T’ | 6 | 2835189 | SERPINB9 | mrna-utr |
| RS318489 | 0.793807 | 0.451046 | 0.684994 | ‘A/G’ | 6 | 2838007 | SERPINB9 | intron |
| RS318491 | 0.139761 | 0.960221 | 0.039025 | ‘A/G’ | 6 | 2843595 | SERPINB9 | intron |
| RS1049269 | 0.151325 | 0.020833 | 0.768675 | ‘A/G’ | 10 | 73490007 | SPOCK2 | mrna-utr |
| RS1613186 | 0.779255 | 0.067304 | 0.166386 | ‘C/T’ | 10 | 73493746 | SPOCK2 | intron |
| RS1678627 | 0.808219 | 0.111262 | 0.228255 | ‘A/G’ | 10 | 73498754 | SPOCK2 | intron |
| RS9663960 | 0.787463 | 0.265568 | 0.427347 | ‘A/G’ | 10 | 73502585 | SPOCK2 | intron |
| RS896074 | 0.145436 | 0.019518 | 0.768675 | ‘A/G’ | 10 | 73502865 | SPOCK2 | intron |
| RS1245560 | 0.190887 | 0.058686 | 0.950398 | ‘G/T’ | 10 | 73507426 | SPOCK2 | intron |
| RS1245548 | 0.491717 | 0.123428 | 0.616667 | ‘C/T’ | 10 | 73515890 | SPOCK2 | intron |
| RS897438 | 0.734944 | 0.787986 | 0.482807 | ‘C/T’ | 18 | 59384104 | SERPINB12 | Intron |
| RS755163 | 0.468498 | 0.22653 | 0.02558 | ‘A/G’ | 18 | 59384115 | SERPINB12 | intron |
| | | | | | | | | locus- |
| RS2983639 | 0.60086 | 0.897697 | 0.493515 | ‘C/T’ | 20 | 23534184 | bA218C14.3 | region |
| | | | | | | | | locus- |
| RS2983640 | 0.030335 | 0.039249 | 0.330703 | ‘A/G’ | 20 | 23534360 | bA218C14.3 | region |

In the Tolloid-like 1 (TLL1) gene, four SNPs had significant associations with HT in men (Table 25). In the SPOCK3 gene, three SNPs in men and four SNPs in women were associated with HT. In addition, one SNP in C5orf5, 6 SNPs in SPINK5, two SNPs in SPINK5L3, one SNP in SERPINB9, two SNPs in SPOCK2, one SNP in SERPINB12, and one SNP in the bA218C14.3 gene had significant association with HT either in all subjects or in men or in women.

### REFERENCES

Abts H F, Welss T, Mirmohammadsadegh A, Kohrer K, Michel G, Ruzicka T. 1999. Cloning and characterization of hurpin (protease inhibitor 13): A new skin-specific, UV-repressible serine proteinase inhibitor of the ovalbumin serpin family. J Mol Biol. 293:29-39.
Alliel P M, Perin J P, Jolles P, Bonnet F J. 1993. Testican, a multidomain testicular proteoglycan resembling modulators of cell social behaviour. Eur J Biochem. 214:347-50.
Altairac S, Zeggai S, Perani P, Courtois Y, Torriglia A. 2003. Apoptosis induced by Na+/H+ antiport inhibition activates the LEI/L-DNase II pathway. Cell Death Differ. 10:548-57.
American Heart Association. Heart Disease and Stroke Statistics-2004 Update. http://www.americanheart.org/downloadable/heart/1079736729696HDSStats2004UpdateRE V3-19-04.pdf (Accessed 17.01.05).
Askew Y S, Pak S C, Luke C J, Askew D J, Cataltepe S, Mills D R, Kato H, Lehoczky J, Dewar K, Birren B, Silverman G A. 2001. SERPINB12 is a novel member of the human ov-serpin family that is widely expressed and inhibits trypsin-like serine proteinases. J Biol Chem. 276: 49320-30.
Barnes R C, Worrall D M. 1995. Identification of a novel human serpin gene; cloning sequencing and expression of leupin. FEBS Lett. 373:61-5.
Barratt J, Feehally J, Smith A C. 2004. Pathogenesis of IgA nephropathy. Semin Nephrol. 24:197-217.
Barrett A J, Rawlings N D and Woessner J F. 1998. Handbook of proteolytic enzymes. Academic Press, San Diego, Calif.
Barrie M B, Stout H W, Abougergi M S, Miller B C, Thiele D L. 2004. Antiviral cytokines induce hepatic expression of the granzyme B inhibitors, proteinase inhibitor 9 and serine proteinase inhibitor 6. J Immunol. 172:6453-9.
BaSalamah M A, Marr H S, Duncan A W, Edgell C J. 2001. Testican in human blood. Biochem Biophys Res Commun. 283:1083-90.
Bitoun E, Micheloni A, Lamant L, Bonnart C, Tartaglia-Polcini A, Cobbold C, Al Saati T, Mariotti F, Mazereeuw-Hautier J, Boralevi F, Hohl D, Harper J, Bodemer C, D'Alessio M, Hovnanian A. 2003. LEKTI proteolytic processing in human primary keratinocytes, tissue distribution and defective expression in Netherton syndrome. Hum Mol Genet. 12:2417-30.
Bladergroen B A, Meijer C J, ten Berge R L, Hack C E, Muris J J, Dukers D F, Chott A, Kazama Y, Oudejans J J, van Berkum O, Kummer J A. 2002. Expression of the granzyme B inhibitor, protease inhibitor 9, by tumor cells in patients with non-Hodgkin and Hodgkin lymphoma: a novel protective mechanism for tumor cells to circumvent the immune system? Blood. 99:232-7.
Bladergroen B A, Strik M C, Wolbink A M, Wouters D, Broekhuizen R, Kummer J A, Hack CE. 2005. The granzyme B inhibitor proteinase inhibitor 9 (PI9) is expressed by human mast cells. Eur J mmunol. 35:1175-83.
Bocock J P, Edgell C J, Marr H S, Erickson A H. 2003. Human proteoglycan testican-1 inhibits the lysosomal cysteine protease cathepsin L. Eur J Biochem. 270:4008-15.
Bonnet F, Perin J P, Charbonnier F, Camuzat A, Roussel G, Nussbaum J L, Alliel P M. 1996. Structure and cellular distribution of mouse brain testican. Association with the postsynaptic area of hippocampus pyramidal cells. J Biol Chem. 271:4373-80.
Bonnet F, Perin J P, Maillet P, Jolles P, Alliel P M. 1992. Characterization of a human seminal plasma glycosaminoglycan-bearing polypeptide. Biochem J. 288:565-9.
Bouton M C, Richard B, Rossignol P, Philippe M, Guillin M C, Michel J B, Jandrot-Perrus M. 2003. The serpin protease-nexin 1 is present in rat aortic smooth muscle cells and is upregulated in L-NAME hypertensive rats. Arterioscler Thromb Vasc Biol. 23:142-7.
Bradshaw A D, Graves D C, Motamed K, Sage E H. 2003. SPARC-null mice exhibit increased adiposity without significant differences in overall body weight. Proc Natl Acad Sci U S A. 100:6045-50.
Brekken R A, Sage E H. 2000. SPARC, a matricellular protein: at the crossroads of cell-matrix. Matrix Biol. 19:569-80.
Buzza M S, Hirst C E, Bird C H, Hosking P, McKendrick J, Bird P I. 2001. The granzyme B inhibitor, PI-9, is present in endothelial and mesothelial cells, suggesting that it protects bystander cells during immune responses. Cell Immunol. 210:21-9.
Chao J, Chai K X, Chen L M, Xiong W, Chao S, Woodley-Miller C, Wang L X, Lu H S, Chao L. 1990. Tissue kallikrein-binding protein is a serpin. I. Purification, characterization, and distribution in normotensive and spontaneously hypertensive rats. J Biol Chem. 265: 16394-401.
Charbonnier F, Chanoine C, Cifuentes-Diaz C, Gallien C L, Rieger F, Alliel P M, Perin J P. 2000. Expression of the proteoglycan SPOCK during mouse embryo development. Mech Dev. 90:317-21.
Charbonnier F, Perin J P, Mattei M G, Camuzat A, Bonnet F, Gressin L, Alliel P M. 1998. Genomic organization of the human SPOCK gene and its chromosomal localization to 5q31. Genomics. 48:377-80.
Cifuentes-Diaz C, Alliel P M, Charbonnier F, de la Porte S, Molgo J, Goudou D, Rieger F, Perin J P. 2000. Regulated expression of the proteoglycan SPOCK in the neuromuscular system. Mech Dev. 94:277-82.
Classen C F, Ushmorov A, Bird P, Debatin K M. 2004. The granzyme B inhibitor PI-9 is differentially expressed in all main subtypes of pediatric acute lymphoblastic leukemias. Haematologica. 89:1314-21.
Cooley J, Mathieu B, Remold-O'Donnell E, Mandle R J. 1998. Production of recombinant human monocyte/neutrophil elastase inhibitor (rM/NEI). Protein Expr Purif. 14:38-44.
Cooley J, Takayama T K, Shapiro S D, Schechter N M, Remold-O'Donnell E. 2001. The serpin MNEI inhibits elastase-like and chymotrypsin-like serine proteases through efficient reactions at two active sites. Biochemistry. 40:15762-70.
Dahlen J R, Foster D C, Kisiel W. 1997. Human proteinase inhibitor 9 (PI9) is a potent inhibitor of subtilisin A. Biochem Biophys Res Commun. 238:329-33.
Dahlen J R, Foster D C, Kisiel W. 1999. Inhibition of neutrophil elastase by recombinant human proteinase inhibitor 9. Biochim Biophys Acta. 1451:233-41.
Darnell J E Jr. 1996. Reflections on STAT3, STAT5, and STAT6 as fat STATs. Proc Natl Acad Sci U S A. 93:6221-4.
De Taeye B, Gils A, Declerck P J. 2004. The story of the serpin plasminogen activator inhibitor 1: is there any need for another mutant? Thromb Haemost. 92: 898-924.
Donahue J E, Berzin T M, Rafli M S, Glass D J, Yancopoulos G D, Fallon J R, Stopa E G. 1999. Agrin in Alzheimer's disease: altered solubility and abnormal distribution within microvasculature and brain parenchyma. Proc Natl Acad Sci U S A. 96:6468-72.
Earnshaw W C, Martins L M, Kaufinann S H. 1999. Mammalian caspases: structure, activation, substrates, and functions during apoptosis. Annu Rev Biochem. 68:383-424.
Empie M W, Laskowski M. 1982. Thermodynamics and kinetics of single residue replacements in avian ovomucoid third domains: effect on inhibitor interactions with serine proteinases. Biochemistry 21: 2274-2284.
Fuentes Rivera, Ricardo Marcelo. 2003. Familial aggregation and tracking of blood pressure, body mass index and serum total cholesterol during childhood. A prospective family study in eastern Finland. Doctoral Dissertation. Kuopio University Publications D. Medical Sciences 296: 139 p. ISBN 951-781-896-3; ISSN 1235-0303.
Garbers D L, Dubois S K.1999. The molecular basis of hypertension. Annu Rev Biochem. 68: 127-55.
Hamada K, Shinomiya H, Asano Y, Kihana T, Iwamoto M, Hanakawa Y, Hashimoto K, Hirose S, Kyo S, Ito M. 2001. Molecular cloning of human squamous cell carcinoma antigen 1 gene and characterization of its promoter. Biochim Biophys Acta. 1518:124-31.
Hartmann U, Maurer P. 2001. Proteoglycans in the nervous system--the quest for functional roles in vivo. Matrix Biol. 20:23-35.
Hasimu B, Nakayama T, Mizutani Y, Izumi Y, Asai S, Soma M, et al. Haplotype analysis of the human renin gene and essential hypertension. Hypertension 2003; 41: 308-12.
Hausser H J, Decking R, Brenner R E. 2004. Testican-1, an inhibitor of pro-MMP-2 activation, is expressed in cartilage. Osteoarthritis Cartilage. 12:870-7.
Inagi R, Miyata T, Nangaku M, Ueyama H, Takeyama K, Kato S, Kurokawa K. 2002. Transcriptional regulation of a mesangium-predominant gene, megsin. J Am Soc Nephrol. 13:2715-22.
Iwasaki M, Nishikawa A, Akutagawa N, Fujimoto T, Teramoto M, Sakaguchi Y, Kato H, Ito M, Yoshida K, Kudo R. 2004. E1AF/PEA3 reduces the invasiveness of SiHa cervical cancer cells by activating serine proteinase inhibitor squamous cell carcinoma antigen. Exp Cell Res. 299:525-32.
Izuhara K. 2003. The role of interleukin-4 and interleukin-13 in the non-immunologic aspects of asthma pathogenesis. Clin Chem Lab Med. 41:860-4.
Janciauskiene S. 2001. Conformational properties of serine proteinase inhibitors (serpins) confer multiple pathophysiological roles. Biochim Biophys Acta. 1535: 221-35.
Jayakumar A, Kang Y, Frederick M J, Pak S C, Henderson Y, Holton P R, Mitsudo K, Silverman G A, EL-Naggar A K, Bromme D, Clayman G L. 2003. Inhibition of the cysteine proteinases cathepsins K and L by the serpin headpin (SERPINB13): a kinetic analysis. Arch Biochem Biophys. 409:367-74.
Jayakumar A, Kang Y, Henderson Y, Mitsudo K, Liu X, Briggs K, Wang M, Frederick M J, El-Naggar A K, Bebok Z, Clayman G L. 2005. Consequences of C-terminal domains and N-terminal signal peptide deletions on LEKTI secretion, stability, and subcellular distribution. Arch Biochem Biophys. 435:89-102.
Kabesch M, Carr D, Weiland S K, von Mutius E. 2004. Association between polymorphisms in serine protease inhibitor, kazal type 5 and asthma phenotypes in a large German population sample. Clin Exp Allergy. 34:340-5.
Kannan-Thulasiraman P, Shapiro D J. 2002. Modulators of inflammation use nuclear factor-kappa B and activator protein-1 sites to induce the caspase- 1 and granzyme B inhibitor, proteinase inhibitor 9. J Biol Chem. 277:41230-9.
Kato A, Fukai K, Oiso N, Hosomi N, Murakami T, Ishii M. 2003. Association of SPINK5 gene polymorphisms with atopic dermatitis in the Japanese population. Br J Dermatol. 148:665-9.
Kotanides H, Reich N C. 1996. Interleukin-4-induced STAT6 recognizes and activates a target site in the promoter of the interleukin-4 receptor gene. J Biol Chem. 271:25555-61.
Krieg S A, Krieg A J, Shapiro D J. 2001. A unique downstream estrogen responsive unit mediates estrogen induction of proteinase inhibitor-9, a cellular inhibitor of IL-1beta-converting enzyme (caspase 1). Mol Endocrinol. 15:1971-82.
Kuwahara F, Kai H, Tokuda K, Shibata R, Kusaba K, Tahara N, Niiyama H, Nagata T, Imaizumi T. 2002. Hypoxia-inducible factor-1alpha/vascular endothelial growth factor pathway for adventitial vasa vasorum formation in hypertensive rat aorta. Hypertension. 39:46-50.
Laskowski M, Kato I, Ardelt W, Cook J, Denton A, Empie M W, Kohr W J, Park S J, Parks K, Schatzley B L, Schoenberger O L, Tashiro M, Vichot G, Whatley H E, Wieczorek A, Wieczorek M. 1987. Ovomucoid third domains from 100 avian species: isolation, sequences, and hypervariability of enzyme-inhibitor contact residues. Biochemistry 26: 202-221.
Li W, Dalen H, Eaton J W, Yuan X M. 2001. Apoptotic death of inflammatory cells in human atheroma. Arterioscler Thromb Vasc Biol. 21:1124-30.
Lopez-Otin C, Overall C M. 2002. Protease degradomics: a new challenge for proteomics.Nat Rev Mol Cell Biol. 3:509-19.
Luft F C. 2001. Mechanisms and cardiovascular damage in hypertension. Hypertension 37:594-598.
Luke C, Schick C, Tsu C, Whisstock J C, Irving J A, Bromme D, Juliano L, Shi G P, Chapman H A, Silverman G A. 2000. Simple modifications of the serpin reactive site loop convert SCCA2 into a cysteine proteinase inhibitor: a critical role for the P3′ proline in facilitating rsL cleavage. Biochemistry. 39:7081-91.
Mahrus S, Kisiel W, Craik C S. 2004. Granzyrne M is a regulatory protease that inactivates proteinase inhibitor 9, an endogenous inhibitor of granzyme B. J Biol Chem. 279:54275-82.
Marr H S, Basalamah M A, Bouldin T W, Duncan A W, Edgell C J. 2000. Distribution of testican expression in human brain. Cell Tissue Res. 302:139-44.
Marr H S, Basalamah M A, Edgell C J. 1997. Endothelial cell expression of testican mRNA. Endothelium. 5:209-19.
Marr H S, Edgell C J. 2003. Testican-1 inhibits attachment of Neuro-2a cells. Matrix Biol. 22:259-66.
McGettrick A F, Barnes R C, Worrall D M. 2001. SCCA2 inhibits TNF-mediated apoptosis in transfected HeLa cells. The reactive centre loop sequence is essential for this function and TNF-induced cathepsin G is a candidate target. Eur J Biochem. 268:5868-75.
Mitsudo K, Jayakumar A, Henderson Y, Frederick M J, Kang Y, Wang M, El-Naggar A K, Clayman G L. 2003. Inhibition of serine proteinases plasmin, trypsin, subtilisin A, cathepsin G, and elastase by LEKTI: a kinetic analysis. Biochemistry. 42:3874-81.
Miyata T, Inagi R, Nangaku M, Imasawa T, Sato M, Izuhara Y, Suzuki D, Yoshino A, Onogi H, Kimura M, Sugiyama S, Kurokawa K. 2002. Overexpression of the serpin megsin induces progressive mesangial cell proliferation and expansion. J Clin Invest. 109:585-93.
Miyata T, Nangaku M, Suzuki D, Inagi R, Uragami K, Sakai H, Okubo K, Kurokawa K. 1998. A mesangium-predominant gene, megsin, is a new serpin upregulated in IgA nephropathy. J Clin Invest. 102:828-36.
Motamed K, Sage E H. 1997. Regulation of vascular morphogenesis by the matricellular protein SPARC. Kidney Int. 51:1383-7.
Moussali H, Bylaite M, Welss T, Abts H F, Ruzicka T, Walz M. 2005. Expression of hurpin, a serine proteinase inhibitor, in normal and pathological skin: overexpression and redistribution in psoriasis and cutaneous carcinomas. Exp Dermatol. 14:420-8.
Mulder N J, Apweiler R, Attwood T K, Bairoch A, Bateman A, Binns D, Bradley P et al. 2005. InterPro, progress and status, 2005.
Naber C K, Siffert W. 2004. Genetics of human arterial hypertension. Minerva Med. 95: 347-56.
Nakada M, Miyamori H, Yamashita J, Sato H. 2003. Testican 2 abrogates inhibition of membrane-type matrix metalloproteinases by other testican family proteins. Cancer Res. 63:3364-9.
Nakada M, Yamada A, Takino T, Miyamori H, Takahashi T, Yamashita J, Sato H. 2001. Suppression of membrane-type 1 matrix metalloproteinase (MMP)-mediated MMP-2 activation and tumor invasion by testican 3 and its splicing variant gene product, N-Tes. Cancer Res. 61:8896-902.
Nakashima T, Pak S C, Silverman G A, Spring P M, Frederick M J, Clayman G L. 2000. Genomic cloning, mapping, structure and promoter analysis of HEADPIN, a serpin which is down-regulated in head and neck cancer cells. Biochim Biophys Acta. 1492:441-6.
Oliveira A S, Xavier-Filho J, Sales M P. 2003. Cysteine Proteinases and Cystatins. Brazilian Archives of Biology and Technology. 46; 91-104.
Puente X S, Sanchez L M, Overall C M, Lopez-Otin C. 2003. Human and mouse proteases: a comparative genomic approach.Nat Rev Genet. 4:544-58.
Quelle F W, Shimoda K, Thierfelder W, Fischer C, Kim A, Ruben S M, Cleveland J L, Pierce J H, Keegan A D, Nelms K, et al.1995. Cloning of murine Stat6 and human Stat6, Stat proteins that are tyrosine phosphorylated in responses to IL-4 and IL-3 but are not required for mitogenesis. Mol Cell Biol. 15:3336-43.
Rawlings N D, Tolle D P, Barrett A J. 2004. Evolutionary families of peptidase inhibitors. Biochem J. 378:705-16.
Rawlings N D, Tolle D P, Barrett A J. 2004. MEROPS: the peptidase database. Nucleic Acids Res. 32 Database issue, D160-D164.
Ray R, Choi M, Zhang Z, Silverman G A, Askew D, Mukherjee A B. 2005. Uteroglobin suppresses SCCA gene expression associated with allergic asthma. J Biol Chem. 280:9761-4.
Remold-O'Donnell E. 1993. The ovalbumin family of serpin proteins. FEBS Lett. 315:105-8.
Rivas M V, Jarvis E D, Morisaki S, Carbonaro H, Gottlieb A B, Krueger J G. 1997. Identification of aberrantly regulated genes in diseased skin using the cDNA differential display technique. J Invest Dermatol. 108:188-94.
Rowshani A T, Florquin S, Bemelman F, Kummer J A, Hack C E, Ten Berge I J. 2004. Hyperexpression of the granzyme B inhibitor PI-9 in human renal allografts: a potential mechanism for stable renal function in patients with subclinical rejection. Kidney Int. 66:1417-22.
Sakaguchi Y, Kishi F, Murakami A, Suminami Y, Kato H. 1999. Structural analysis of human SCC antigen 2 promoter. Biochim Biophys Acta. 1444:111-6.
Sakata Y, Arima K, Takai T, Sakurai W, Masumoto K, Yuyama N, Suminami Y, Kishi F, Yamashita T, Kato T, Ogawa H, Fujimoto K, Matsuo Y, Sugita Y, Izuhara K. 2004. The squamous cell carcinoma antigen 2 inhibits the cysteine proteinase activity of a major mite allergen, Der p 1. J Biol Chem. 279:5081-7.
Salonen J T, Lakka T A, Lakka H-M, Valkonen V-P, Everson S A, Kaplan G A. 1998. Hyperinsulinemia is associated with the Incidence of hypertension and dyslipidemia in middle-aged men. Diabetes 47: 270-275.
Salonen J T, Puska P, Kottke T E, Heinonen O P. 1981. Coronary risk factor clustering patterns in Eastern Finland. Int J Epidemiol 10: 203-210.
Schick C, Kamachi Y, Bartuski A J, Cataltepe S, Schechter N M, Pemberton P A, Silverman G A. 1997. Squamous cell carcinoma antigen 2 is a novel serpin that inhibits the chymotrypsin-like proteinases cathepsin G and mast cell chymase. J Biol Chem. 272:1849-55.
Schick C, Pemberton P A, Shi G P, Kamachi Y, Cataltepe S, Bartuski A J, Gornstein E R, Bromme D, Chapman H A, Silverman G A. 1998. Cross-class inhibition of the cysteine proteinases cathepsins K, L, and S by the serpin squamous cell carcinoma antigen 1: a kinetic analysis. Biochemistry. 37:5258-66.
Schneider S S, Schick C, Fish K E, Miller E, Pena J C, Treter S D, Hui S M, Silverman G A. 1995. A serine proteinase inhibitor locus at 18q21.3 contains a tandem duplication of the human squamous cell carcinoma antigen gene. Proc Natl Acad Sci U S A. 92:3147-51.
Schnepp A, Komp Lindgren P, Hulsmann H, Kroger S, Paulsson M, Hartmann U. 2005. Mouse testican-2. Expression, glycosylation, and effects on neurite outgrowth. J Biol Chem. 280:11274-80.
Scott F L, Eyre H J, Lioumi M, Ragoussis J, Irving J A, Sutherland G A, Bird P I. 1999. Human ovalbumin serpin evolution: phylogenic analysis, gene organization, and identification of new P18-related genes suggest that two interchromosomal and several intrachromosomal duplications generated the gene clusters at 18q21-q23 and 6p25. Genomics. 62: 490-9.
Scott I C, Blitz I L, Pappano W N, Imamura Y, Clark T G, Steiglitz B M, Thomas C L, Maas S A, Takahara K, Cho K W Y, Greenspan D S. 1999. Mammalian BMP-1/tolloid-related metalloproteinases, including novel family member mammalian tolloid-like 2, have differential enzymatic activities and distributions of expression relevant to patterning and skeletogenesis. Dev Biol 213: 283-300.
Sharma A M, Chetty V T. 2005. Obesity, hypertension and insulin resistance. Acta Diabetol. 42 Suppl 1:S3-8.
Shiffman D, Somogyi R, Lawn R, Seilhamer J J, Porter J G, Mikita T, Tai J. 2003. Genes expressed in foam cell differentiation. United States Patent Application US 20030165924.
Silverman G A, Bird P I, Carrell R W, Church F C, Coughlin P B, Gettins P G, Irving J A, Lomas D A, Luke C J, Moyer R W, Pemberton P A, Remold-O'Donnell E, Salvesen G S, Travis J, Whisstock J C. 2001. The serpins are an expanding superfamily of structurally similar but functionally diverse proteins. Evolution, mechanism of inhibition, novel functions, and a revised nomenclature. J Biol Chem. 276: 33293-6.
Southan C. 2001. A genomic perspective on human proteases. FEBS Lett. 498:214-8. Sprecher C A, Morgenstern K A, Mathewes S, Dahlen J R, Schrader S K, Foster D C, Kisiel W. 1995. Molecular cloning, expression, and partial characterization of two novel members of the ovalbumin family of serine proteinase inhibitors. J Biol Chem. 270:29854-61.
Staessen J A, Wang J, Bianchi G, Birkenhager W H. 2003. Essential hypertension. Lancet. 361:1629-41.
Stark M, Danielsson O, Griffiths W J, Jornvall H, Johansson J. 2001. Peptide repertoire of human cerebrospinal fluid: novel proteolytic fragments of neuroendocrine proteins. J Chromatogr B Biomed Sci Appl. 754:357-67.
Steinerova A, Racek J, Stozicky F, Zima T, Fialova L, Lapin A. 2001. Antibodies against oxidized LDL—theory and clinical use. Physiol Res. 50:131-41.
Steinhoff M, Buddenkotte J, Shpacovitch V, Rattenholl A, Moormann C, Vergnolle N, Luger T A, Hollenberg M D. 2005. Proteinase-Activated Receptors: Transducers of Proteinase-Mediated Signaling in Inflammation and Immune Response. Endocr Rev. 26:1-43.
Sukhova G K, Shi G P, Simon D I, Chapman H A, Libby P. 1998. Expression of the elastolytic cathepsins S and K in human atheroma and regulation of their production in smooth muscle cells. J Clin Invest. 102:576-83.
Suminami Y, Kishi F, Nawata S, Murakami A, Sakaguchi Y, Sueoka K, Numa F, Sugino N, Kato H. 2005. Promoter analyses of SCC antigen genes. Biochim Biophys Acta. 1727:208-12.
Sun J, Bird C H, Sutton V, McDonald L, Coughlin P B, De Jong T A, Trapani J A, Bird P I. 1996. A cytosolic granzyme B inhibitor related to the viral apoptotic regulator cytokine response modifier A is present in cytotoxic lymphocytes. J Biol Chem. 271:27802-9.
Takeda A, Higuchi D, Takahashi T, Ogo M, Baciu P, Goetinck P F, Hibino T. 2002. Overexpression of serpin squamous cell carcinoma antigens in psoriatic skin. J Invest Dermatol. 118:147-54.
Thakker-Varia S, Elkabes S, Schick C, Silverman G A, Peng L, Sherwood A C, Black I B. 1998. Gene expression in activated brain microglia: identification of a proteinase inhibitor that increases microglial cell number. Brain Res Mol Brain Res. 56:99-107.
Thornberry N A, Lazebnik Y. 1998. Caspases: enemies within. Science. 281:1312-6.
Torriglia A, Perani P, Brossas J Y, Chaudun E, Treton J, Courtois Y, Counis M F. 1998. L-DNase II, a molecule that links proteases and endonucleases in apoptosis, derives from the ubiquitous serpin leukocyte elastase inhibitor. Mol Cell Biol. 18:3612-9.
Tsen G, Halfter W, Kroger S, Cole G J. 1995. Agrin is a heparan sulfate proteoglycan. J Biol Chem. 270:3392-9.
Walley A J, Chavanas S, Moffatt M F, Esnouf R M, Ubhi B, Lawrence R, Wong K, Abecasis G R, Jones E Y, Harper J I, Hovnanian A, Cookson W O. 2001. Gene polymorphism in Netherton and common atopic disease. Nat Genet. 29:175-8.
van Gent D, Sharp P, Morgan K, Kalsheker N. 2003. Serpins: structure, function and molecular evolution. Int J Biochem Cell Biol. 35: 1536-47.
Wang J, Xu J, Finnerty J, Furuta M, Steiner D F, Verchere C B. 2001. The prohormone convertase enzyme 2 (PC2) is essential for processing pro-islet amyloid polypeptide at the NH2-terminal cleavage site. Diabetes. 50:534-9.
Wex T, Lipyansky A, Bromme N C, Wex H, Guan X Q, Bromme D. 2001. TIN-ag-RP, a novel catalytically inactive cathepsin B-related protein with EGF domains, is predominantly expressed in vascular smooth muscle cells. Biochemistry. 40:1350-7.
Williamson M P, Marion D, Wuthrich K. 1984. Secondary structure in the solution conformation of the proteinase inhibitor IIA from bull seminal plasma by nuclear magnetic resonance. J. Mol. Biol. 173: 341-359.
Woods D E, Cantin A, Cooley J, Kenney D M, Remold-O'Donnell E. 2005. Aerosol treatment with MNEI suppresses bacterial proliferation in a model of chronic Pseudomonas aeruginosa lung infection. Pediatr Pulmonol. 39:141-9.
Young J L, Sukhova G K, Foster D, Kisiel W, Libby P, Schonbeck U. 2000. The serpin proteinase inhibitor 9 is an endogenous inhibitor of interleukin 1beta-converting enzyme (caspase-1) activity in human vascular smooth muscle cells. J Exp Med. 191:1535-44.
Zeng W, Remold-O'Donnell E. 2000. Human monocyte/neutrophil elastase inhibitor (MNEI) is regulated by PU.1/Spi-1, Sp1, and NF-kappaB. J Cell Biochem. 78:519-32.
Zeng W, Silverman G A, Remold-O'Donnell E. 1998. Structure and sequence of human M/NEI (monocyte/neutrophil elastase inhibitor), an Ov-serpin family gene. Gene. 213:179-87.

1. A method for preventing or treating a cardiovascular or metabolic condition or related trait in a mammalian subject comprising one or more agents modulating the activity of biological networks and/or metabolic pathways comprising ov-SERPIN, SPINK and/or SPOCK genes, or corresponding RNAs, proteins and polypeptides.

2. The method according to claim 1, wherein a cardiovascular or metabolic condition or trait is selected from the group consisting of ischemia, ischemic tissue damage, myocardial damage, blood pressure regulation, lipid metabolism, glucose metabolism, energy metabolism or appetite regulation.

3. The method according to claim 1, wherein the cardiovascular condition is a cardiovascular disease such as coronary heart disease or hypertension.

4. The method according to claim 3, wherein coronary heart disease may manifest as either coronary death, myocardial infarction, angina pectoris or other chronic coronary heart disease.

5. The method according to claim 1, wherein the metabolic condition is the metabolic syndrome, obesity, or lipid disorder.

6. The method according to claim 5, wherein the lipid disorder comprises altered plasma concentration of lipoproteins such as low high density lipoprotein, elevated very low density lipoprotein, elevated low density lipoprotein, elevated apolipoprotein (a), elevated triglycerides or elevated cholesterol.

7. The method according to claim 1, wherein the subject is at elevated risk of cardiovascular or metabolic disease because of family history.

8. The method according to claim 1, wherein the subject has atopic conditions, a skin disease or family history of said conditions.

9. The method according to claim 1, wherein the subject has susceptibility to infectious diseases.

10. The method according to claim 1, wherein said biological networks and metabolic pathways are related to fibrinolysis, coagulation, endothelial dysfunction, inflammation, inflammatory response, cell mobility, cellular differentiation, extracellular matrix remodeling, cellular death, cellular transport, peptidase activity, polypeptide aggregation, polypeptide cleavage or proteolysis.

11. The method according to claim 1, wherein said biological networks and metabolic pathways are related to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides.

12. The method according to claim 1 comprising administering to a mammalian subject in need of such treatment an effective amount of a compound in a pharmaceutically acceptable carrier enhancing or reducing biological activity or availability of one or more polypeptides encoded by ov-SERPIN, SPINK and SPOCK genes.

13. The method according to claim 1 comprising administering to a mammalian subject in need of such treatment an effective amount of a compound in a pharmaceutically acceptable carrier enhancing or reducing biological activity or availability of one or more polypeptides encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes.

14. The method according to claim 1 comprising administering to a mammalian subject in need of such treatment an effective amount of a compound in a pharmaceutically acceptable carrier enhancing or reducing expression of one or more genes selected from ov-SERPIN, SPINK and SPOCK genes.

15. The method according to claim 1 comprising administering to a mammalian subject in need of such treatment an effective amount of a compound in a pharmaceutically acceptable carrier enhancing or reducing expression of one or more genes selected from SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes.

16. The method according to claim 1, wherein said agents enhance or reduce expression of one or more genes in biological networks and/or metabolic pathways comprising ov-SERPIN, SPINK and SPOCK genes, RNAs, proteins and polypeptides.

17. The method according to claim 1, wherein said agents enhance or reduce expression of one or more genes in biological networks and/or metabolic pathways comprising SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides.

18. The method according to claim 1, wherein said agents enhance or reduce expression of one or more genes containing a sparc/osteonectin-domain, a follistatin-domain or a kazal-domain.

19. The method according to claim 1, wherein said agents enhance or reduce activity of one or more pathophysiological pathways comprising ov-SERPIN, SPINK and/or SPOCK genes, RNAs, proteins and polypeptides.

20. The method according to claim 1, wherein said agents enhance or reduce activity of one or more pathophysiological pathways comprising SERPNB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides.

21. The method according to claim 1, wherein said agents comprise ov-SERPIN, SPINK and/or SPOCK genes, RNAs, proteins and polypeptides, and their active fragments and derivatives thereof.

22. The method according to claim 1, wherein said agents comprise SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides, and their active fragments and derivatives thereof.

23. The method according to claim 1 comprising gene therapy or gene transfer.

24. The method according to claim 23 comprising treating regulatory regions and/or polypeptide encoding regions of one or more genes related to said biological networks and/or metabolic pathways in somatic cells or stem cells of said subject.

25. The method according to claim 1 comprising sequence specific gene silencing agents such as siRNA hybridising to mRNA and/or to hnRNA of one or more genes related to said biological networks and/or metabolic pathways.

26. The method according to claim 1 comprising dietary treatment or a vaccination.

27. A method for risk prediction, diagnosis or prognosis of a cardiovascular or metabolic condition or trait comprising the steps of:
a) providing a biological sample taken from the subject;
b) assessing type and/or level of one or more biomarkers in said sample, wherein said biomarkers are associated to biological networks and/or metabolic pathways comprising ov-SERPIN, SPINK and/or SPOCK genes, or corresponding RNAs, proteins and polypeptides; and
c) comparing the biomarker data from the subject to the biomarker data from samples representing healthy and/or diseased individuals to make risk prediction, diagnosis or prognosis of a cardiovascular or metabolic condition.

28. The method according to claim 27, wherein a cardiovascular or metabolic condition or trait is selected from the group consisting of ischemia, ischemic tissue damage, myocardial damage, blood pressure regulation, lipid metabolism, glucose metabolism, energy metabolism or appetite regulation.

29. The method according to claim 27, wherein the cardiovascular condition is a cardiovascular disease such as coronary heart disease or hypertension.

30. The method according to claim 29, wherein coronary heart disease may manifest as coronary death, myocardial infarction, angina pectoris or other chronic coronary heart disease.

31. The method according to claim 27, wherein the metabolic condition is the metabolic syndrome, obesity, or lipid disorder.

32. The method according to claim 31, wherein the lipid disorder comprises altered plasma concentration of lipoproteins such as low high density lipoprotein, elevated very low density lipoprotein, elevated low density lipoprotein, elevated apolipoprotein (a), elevated triglycerides or elevated cholesterol.

33. The method according to claim 27, wherein the subject is at elevated risk of cardiovascular or metabolic disease because of family history.

34. The method according to claim 27, wherein the subject has atopic conditions, a skin disease or family history of said conditions.

35. The method according to claim 27, wherein the subject has susceptibility to infectious diseases.

36. The method according to claim 27, wherein said biological networks and metabolic pathways are related to fibrinolysis, coagulation, endothelial dysfunction, inflammation, inflammatory response, cell mobility, cellular differentiation, extracellular matrix remodeling, cellular death, cellular transport, peptidase activity, polypeptide aggregation, polypeptide cleavage or proteolysis.

37. The method according to claim 27, wherein said biological networks and metabolic pathways comprise SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides.

38. The method according to claim 27, wherein said biomarkers are associated to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides.

39. The method according to claim 27, wherein said biomarkers are selected from ov-SERPIN, SPINK and SPOCK genes, RNAs, proteins and polypeptides.

40. The method according to claim 27, wherein said biomarkers are selected from SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes, RNAs, proteins and polypeptides.

41. The method according to claim 27, wherein said biomarkers are selected from polymorphic sites residing in genomic regions containing ov-SERPIN, SPINK and SPOCK genes.

42. The method according to claim 27, wherein said biomarkers are selected from polymorphic sites residing in genomic regions containing SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes.

43. The method according to claim 27, wherein said biomarkers are selected from SNP markers set forth in tables 1 to 20 and 23 to 25.

44. The method according to claim 43, wherein said biomarkers are polymorphic sites associated with one or more of the SNP markers set forth in tables 1 to 20 and 23 to 25.

45. The method according to claim 43, wherein said biomarkers are polymorphic sites being in complete linkage disequilibrium with one or more of the SNP markers set forth in tables 1 to 20 and23 to 25.

46. The method according to claim 27, wherein said biomarkers are associated to genes containing a sparc/osteonectin-domain, a follistatin-domain or a kazal-domain.

47. The method according to claim 27 for monitoring the effect of a therapy administered to a subject having a cardiovascular or metabolic condition.

48. The method according to claim 27 for selecting efficient and safe therapy for a subject having a cardiovascular or metabolic condition.

49. The method according to claim 27 for diagnosing a subtype of a cardiovascular or metabolic condition in a subject having a cardiovascular or metabolic condition.

50. The method according to claim 27 for predicting the effectiveness of a given therapeutic to treat a cardiovascular or metabolic condition or trait in a subject having a cardiovascular or metabolic condition.

51. The method according to claim 27 for selecting efficient and safe preventative therapy to a subject having increased risk of a cardiovascular or metabolic condition.

52. The method according to claim 27 for monitoring the effect of a preventive therapy administered to a subject having increased risk of a cardiovascular or metabolic condition.

53. The method according to claim 27 for predicting the effectiveness of a given therapeutic to prevent a cardiovascular or metabolic condition in a subject having increased risk of a cardiovascular or metabolic condition.

54. The method according to claim 27 for selecting subjects for clinical trials.

55. The method according to claim 27 further comprising step d) combining personal and clinical information with the biomarker data to make risk prediction, diagnosis or prognosis of a cardiovascular or metabolic condition.

56. The method according to claim 55, wherein the personal and clinical information comprises concerns age, gender, socioeconomic measurements, psychological traits and states, behaviour patterns and habits, biochemical measurements, clinical measurements, anthropometric measurements and obesity, the family history of hypertension, coronary heart disease disease, other cardiovascular disease, hypercholesterolemia, obesity and diabetes, and the medical history of the subject.

57. A test kit for risk prediction, diagnosis or prognosis of a cardiovascular or metabolic condition or trait comprising:
a) reagents, materials and protocols for assessing type and/or level of one or more biomarkers in a biological sample, wherein said biomarkers are associated to biological networks and/or metabolic pathways comprising ov-SERPIN, SPINK and/or SPOCK genes, or corresponding RNAs, proteins and polypeptides; and
b) instructions and software for comparing the biomarker data from the subject to the biomarker data from samples representing healthy and/or diseased individuals to make risk prediction, diagnosis or prognosis of a cardiovascular or metabolic condition.

58. The test kit according to claim 57, wherein a cardiovascular or metabolic condition or trait is selected from the group consisting of ischemia, ischemic tissue damage, myocardial damage, blood pressure regulation, lipid metabolism, glucose metabolism, energy metabolism or appetite regulation.

59. The test kit according to claim 57, wherein the cardiovascular condition is a cardiovascular disease such as coronary heart disease or hypertension.

60. The test kit according to claim 59, wherein coronary heart disease may manifest as coronary death, myocardial infarction, angina pectoris or other chronic coronary heart disease.

61. The test kit according to claim 57, wherein the metabolic condition is the metabolic syndrome, obesity, or lipid disorder.

62. The test kit according to claim 61, wherein the lipid disorder comprises altered plasma concentration of lipoproteins such as low high density lipoprotein, elevated very low density lipoprotein, elevated low density lipoprotein, elevated apolipoprotein (a), elevated triglycerides or elevated cholesterol.

63. The test kit according to claim 57, wherein said biological networks and metabolic pathways are related to fibrinolysis, coagulation, endothelial dysfunction, inflammation, inflammatory response, cell mobility, cellular differentiation, extracellular matrix remodeling, cellular death, cellular transport, peptidase activity, polypeptide aggregation, polypeptide cleavage and proteolysis.

64. The test kit according to claim 57, wherein said biological networks and metabolic pathways comprise SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides.

65. The test kit according to claim 57, wherein said biomarkers are associated to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides.

66. The test kit according to claim 57, wherein said biomarkers are selected from ov-SERPIN, SPINK and SPOCK genes, RNAs, proteins and polypeptides.

67. The test kit according to claim 57, wherein said biomarkers are selected from SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes, RNAs, proteins and polypeptides.

68. The test kit according to claim 57, wherein said biomarkers are selected from polymorphic sites residing in genomic regions containing ov-SERPIN, SPINK and SPOCK genes.

69. The test kit according to claim 57, wherein said biomarkers are selected from polymorphic sites residing in genomic regions containing SERPINB1, SERPINB2, SERPINB3, SERPINB4, SERPINB5, SERPINB7, SEPRINB8, SERPINB9, SERPINB11, SERPINB12, SERPINB3, SPINK5, SPINK5L2, SPINK5L3, SPOCK, SPOCK2, TLL1 and SPOCK3 genes.

70. The test kit according to claim 57, wherein said biomarkers are selected from SNP markers set forth in tables 1 to 20 and 23 to 25.

71. The test kit according to claim 70, wherein said biomarkers are polymorphic sites associated with one or more of the SNP markers set forth in tables 1 to 20 and 23 to 25.

72. The test kit according to claim 70, wherein said biomarkers are polymorphic sites being in complete linkage disequilibrium with one or more of the SNP markers set forth in tables 1 to 20 and 23 to 25.

73. The test kit according to claim 57, wherein said biomarkers are associated to genes containing a sparc/osteonectin-domain, a follistatin-domain or a kazal-domain.

74. The test kit according to claim 57 for monitoring the effect of a therapy administered to a subject having a cardiovascular or metabolic condition.

75. The test kit according to claim 57 for selecting efficient and safe therapy for a subject having a cardiovascular or metabolic condition.

76. The test kit according to claim 57 for diagnosing a subtype of a cardiovascular or metabolic condition in a subject having a cardiovascular or metabolic condition.

77. The test kit according to claim 57 for predicting the effectiveness of a given therapeutic to treat a cardiovascular or metabolic condition or trait in a subject having a cardiovascular or metabolic condition.

78. The test kit according to claim 57 for selecting efficient and safe preventative therapy to a subject having increased risk of a cardiovascular or metabolic condition.

79. The test kit according to claim 57 for monitoring the effect of a preventive therapy administered to a subject having increased risk of a cardiovascular or metabolic condition.

80. The test kit according to claim 57 for predicting the effectiveness of a given therapeutic to prevent a cardiovascular or metabolic condition in a subject having increased risk of a cardiovascular or metabolic condition.

81. The test kit according to claim 57 for selecting subjects for clinical trials.

82. The test kit according to claim 57 further comprising questionnaire and instructions for collecting personal and clinical information from the subject.

83. The method according to claim 83, wherein the personal and clinical information comprises concerns age, gender, socioeconomic measurements, psychological traits and states, behaviour patterns and habits, biochemical measurements, clinical measurements, anthropometric measurements and obesity, the family history of hypertension, coronary heart disease disease, other cardiovascular disease, hypercholesterolemia, obesity and diabetes, and the medical history of the subject.

84. A method for screening agents for preventing, treating or reducing the risk of a cardiovascular or metabolic condition in a mammal by determining the effect of agents on biological networks and/or metabolic pathways comprising ov-SERPIN, SPINK and/or SPOCK genes, or corresponding RNAs, proteins and polypeptides in living cells; wherein an agent altering activity of one or several said biological networks and/or metabolic pathways is considered useful in preventing, treating or reducing the risk of a cardiovascular or metabolic condition.

85. The method according to claim 84, wherein said biological networks and metabolic pathways comprise SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides.

86. The method according to claim 84, wherein said biological networks and metabolic pathways are related to fibrinolysis, coagulation, endothelial dysfunction, inflammation, inflammatory response, cell mobility, cellular differentiation, extracellular matrix remodeling, cellular death, cellular transport, peptidase activity, polypeptide aggregation, polypeptide cleavage or proteolysis.

87. The method according to claim 84 comprising non-human transgenic animals, mammalian tissues, organs or organ systems, or cultured microbial, insect or mammalian cells expressing one or more of the SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes.

88. A pharmaceutical composition for preventing or treating a cardiovascular or metabolic condition or related trait in a mammalian subject comprising one or more agents in a pharmaceutically acceptable carrier modulating the activity of biological networks and/or metabolic pathways comprising ov-SERPIN, SPINK and/or SPOCK genes, or corresponding RNAs, proteins and polypeptides.

89. The pharmaceutical composition according to claim 88, wherein said biological networks and metabolic pathways are related to SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, or corresponding RNAs, proteins and polypeptides.

90. The pharmaceutical composition according to claim 88, wherein said biological networks and metabolic pathways are related to fibrinolysis, coagulation, endothelial dysfunction, inflammation, inflammatory response, cell mobility, cellular differentiation, extracellular matrix remodeling, cellular death, cellular transport, peptidase activity, polypeptide aggregation, polypeptide cleavage or proteolysis.

91. The pharmaceutical composition according to claim 88, wherein said agents comprise ov-SERPIN, SPINK and/or SPOCK genes, or corresponding RNAs, proteins or polypeptides, and their active fragments and derivatives thereof.

92. The pharmaceutical composition according to claim 88, wherein said agents comprise SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides, and their active fragments and derivatives thereof.

93. The pharmaceutical composition according to claim 88 comprising one or more agents enhancing or reducing biological activity or availability of one or more polypeptides encoded by ov-SERPIN, SPINK and SPOCK genes.

94. The pharmaceutical composition according to claim 88 comprising one or more agents enhancing or reducing biological activity or availability of one or more polypeptides encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes.

95. The pharmaceutical composition according to claim 88, wherein said agents enhance or reduce expression of one or more genes in biological networks and/or metabolic pathways and/or pathophysiological pathways comprising ov-SERPIN, SPINK and SPOCK genes, RNAs, proteins and polypeptides.

96. The pharmaceutical composition according to claim 88, wherein said agents enhance or reduce expression of one or more genes in biological networks and/or metabolic pathways and/or pathophysiological pathways comprising SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and/or SPOCK3 genes, RNAs, proteins and polypeptides.

97. The pharmaceutical composition according to claim 88 comprising one or more agents restoring, at least partially, the observed alterations in biological activity of one or more proteins and polypeptides encoded by ov-SERPIN, SPINK and SPOCK genes in said subject, when compared to healthy subjects.

98. The pharmaceutical composition according to claim 88 comprising one or more agents restoring, at least partially, the observed alterations in biological activity of one or more proteins and polypeptides encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3 genes in said subject, when compared to healthy subjects.

99. The pharmaceutical composition according to claim 88 comprising one or more agent binding to one or more proteins and polypeptides encoded by ov-SERPIN, SPINK and SPOCK genes.

100. The pharmaceutical composition according to claim 88 comprising one or more agent binding to one or more proteins and polypeptides encoded by SERPINB1, SERPINB2, SERPINB3, SERPINB4, SERPINB5, SERPINB7, SEPRINB8, SERPINB9, SERPINB11, SERPINB12, SERPINB13, SPINK5, SPINK5L2, SPINK5L3, SPOCK, SPOCK2, TLL1 and SPOCK3.

101. A kit for preventing, treating or reducing the risk of a cardiovascular or metabolic condition in a mammalian subject comprising a pharmaceutical composition according to claim 88 and instructions for use.
